SEQUENCE LISTING (448 sequences)

SEQ ID NO: 1 - protein, 6 aa, Artificial Sequence
  Description of Artificial Sequence Synthetic mimotope peptide
EIDYHR

SEQ ID NO: 2 - protein, 6 aa, Artificial Sequence
  Description of Artificial Sequence Synthetic mimotope peptide
ELDYHR

SEQ ID NO: 3 - protein, 6 aa, Artificial Sequence
  Description of Artificial Sequence Synthetic mimotope peptide
EVDYHR

SEQ ID NO: 4 - protein, 6 aa, Artificial Sequence
  Description of Artificial Sequence Synthetic mimotope peptide
DIDYHR

SEQ ID NO: 5 - protein, 6 aa, Artificial Sequence
  Description of Artificial Sequence Synthetic mimotope peptide
DLDYHR

SEQ ID NO: 6 - protein, 6 aa, Artificial Sequence
  Description of Artificial Sequence Synthetic mimotope peptide
DVDYHR

SEQ ID NO: 7 - protein, 6 aa, Artificial Sequence
  Description of Artificial Sequence Synthetic mimotope peptide
DIDYRR

SEQ ID NO: 8 - protein, 6 aa, Artificial Sequence
  Description of Artificial Sequence Synthetic mimotope peptide
DLDYRR

SEQ ID NO: 9 - protein, 6 aa, Artificial Sequence
  Description of Artificial Sequence Synthetic mimotope peptide
DVDYRR

SEQ ID NO: 10 - protein, 6 aa, Artificial Sequence
  Description of Artificial Sequence Synthetic mimotope peptide
DKELRI

SEQ ID NO: 11 - protein, 6 aa, Artificial Sequence
  Description of Artificial Sequence Synthetic mimotope peptide
DWELRI

SEQ ID NO: 12 - protein, 6 aa, Artificial Sequence
  Description of Artificial Sequence Synthetic mimotope peptide
YREFRI

SEQ ID NO: 13 - protein, 6 aa, Artificial Sequence
  Description of Artificial Sequence Synthetic mimotope peptide
YAEFRG

SEQ ID NO: 14 - protein, 6 aa, Artificial Sequence
  Description of Artificial Sequence Synthetic mimotope peptide
EAEFRG

SEQ ID NO: 15 - protein, 6 aa, Artificial Sequence
  Description of Artificial Sequence Synthetic mimotope peptide
DYEFRG

SEQ ID NO: 16 - protein, 6 aa, Artificial Sequence
  Description of Artificial Sequence Synthetic mimotope peptide
ELEFRG

SEQ ID NO: 17 - protein, 6 aa, Artificial Sequence
  Description of Artificial Sequence Synthetic mimotope peptide
DRELRI

SEQ ID NO: 18 - protein, 6 aa, Artificial Sequence
  Description of Artificial Sequence Synthetic mimotope peptide
DKELKI

SEQ ID NO: 19 - protein, 6 aa, Artificial Sequence
  Description of Artificial Sequence Synthetic mimotope peptide
DRELKI

SEQ ID NO: 20 - protein, 6 aa, Artificial Sequence
  Description of Artificial Sequence Synthetic mimotope peptide
GREFRN

SEQ ID NO: 21 - protein, 6 aa, Artificial Sequence
  Description of Artificial Sequence Synthetic mimotope peptide
EYEFRG

SEQ ID NO: 22 - protein, 7 aa, Artificial Sequence
  Description of Artificial Sequence Synthetic mimotope peptide
DWEFRDA

SEQ ID NO: 23 - protein, 6 aa, Artificial Sequence
  Description of Artificial Sequence Synthetic mimotope peptide
SWEFRT

SEQ ID NO: 24 - protein, 5 aa, Artificial Sequence
  Description of Artificial Sequence Synthetic mimotope peptide
DKELR

SEQ ID NO: 25 - protein, 6 aa, Artificial Sequence
  Description of Artificial Sequence Synthetic mimotope peptide
SFEFRG

SEQ ID NO: 26 - protein, 6 aa, Homo sapiens
DAEFRH

SEQ ID NO: 27 - protein, 7 aa, Artificial Sequence
  Description of Artificial Sequence Synthetic peptide
DAEFRWP

SEQ ID NO: 28 - protein, 7 aa, Artificial Sequence
  Description of Artificial Sequence Synthetic peptide
DNEFRSP

SEQ ID NO: 29 - protein, 7 aa, Artificial Sequence
  Description of Artificial Sequence Synthetic peptide
GSEFRDY

SEQ ID NO: 30 - protein, 7 aa, Artificial Sequence
  Description of Artificial Sequence Synthetic peptide
GAEFRFT

SEQ ID NO: 31 - protein, 7 aa, Artificial Sequence
  Description of Artificial Sequence Synthetic peptide
SAEFRTQ

SEQ ID NO: 32 - protein, 7 aa, Artificial Sequence
  Description of Artificial Sequence Synthetic peptide
SAEFRAT

SEQ ID NO: 33 - protein, 7 aa, Artificial Sequence
  Description of Artificial Sequence Synthetic peptide
SWEFRNP

SEQ ID NO: 34 - protein, 7 aa, Artificial Sequence
  Description of Artificial Sequence Synthetic peptide
SWEFRLY

SEQ ID NO: 35 - protein, 7 aa, Artificial Sequence
  Description of Artificial Sequence Synthetic peptide
SWELRQA

SEQ ID NO: 36 - protein, 7 aa, Artificial Sequence
  Description of Artificial Sequence Synthetic peptide
SVEFRYH

SEQ ID NO: 37 - protein, 7 aa, Artificial Sequence
  Description of Artificial Sequence Synthetic peptide
SYEFRHH

SEQ ID NO: 38 - protein, 7 aa, Artificial Sequence
  Description of Artificial Sequence Synthetic peptide
SQEFRTP

SEQ ID NO: 39 - protein, 7 aa, Artificial Sequence
  Description of Artificial Sequence Synthetic peptide
SSEFRVS

SEQ ID NO: 40 - protein, 6 aa, Artificial Sequence
  Description of Artificial Sequence Synthetic peptide
DWEFRD

SEQ ID NO: 41 - protein, 6 aa, Artificial Sequence
  Description of Artificial Sequence Synthetic peptide
DAELRY

SEQ ID NO: 42 - protein, 6 aa, Artificial Sequence
  Description of Artificial Sequence Synthetic peptide
DWELRQ

SEQ ID NO: 43 - protein, 6 aa, Artificial Sequence
  Description of Artificial Sequence Synthetic peptide
SLEFRF

SEQ ID NO: 44 - protein, 6 aa, Artificial Sequence
  Description of Artificial Sequence Synthetic peptide
GPEFRW

SEQ ID NO: 45 - protein, 6 aa, Artificial Sequence
  Description of Artificial Sequence Synthetic peptide
GKEFRT

SEQ ID NO: 46 - protein, 6 aa, Artificial Sequence
  Description of Artificial Sequence Synthetic peptide
AYEFRH

SEQ ID NO: 47 - protein, 5 aa, Artificial Sequence
  Description of Artificial Sequence Synthetic peptide
DKELR

SEQ ID NO: 48 - protein, 5 aa, Artificial Sequence
  Description of Artificial Sequence Synthetic peptide
DKEVR

SEQ ID NO: 49 - protein, 5 aa, Artificial Sequence
  Description of Artificial Sequence Synthetic peptide
DKEAR

SEQ ID NO: 50 - protein, 11 aa, Homo sapiens
SEVKMDAEFRH

SEQ ID NO: 51 - protein, 6 aa, Artificial Sequence
  Description of Artificial Sequence Synthetic mimotope peptide
WHWSWR

SEQ ID NO: 52 - protein, 6 aa, Artificial Sequence
  Description of Artificial Sequence Synthetic mimotope peptide
KKELRI

SEQ ID NO: 53 - protein, 7 aa, Artificial Sequence
  Description of Artificial Sequence Synthetic peptide
VPTSALA

SEQ ID NO: 54 - protein, 7 aa, Artificial Sequence
  Description of Artificial Sequence Synthetic peptide
ATYAYWN

SEQ ID NO: 55 - protein, 7 aa, Artificial Sequence
  Description of Artificial Sequence Synthetic mimotope peptide
IRWDTPC

SEQ ID NO: 56 - protein, 8 aa, Artificial Sequence
  Description of Artificial Sequence Synthetic mimotope peptide
VRWDVYPC

SEQ ID NO: 57 - protein, 8 aa, Artificial Sequence
  Description of Artificial Sequence Synthetic mimotope peptide
IRYDAPLC

SEQ ID NO: 58 - protein, 8 aa, Artificial Sequence
  Description of Artificial Sequence Synthetic mimotope peptide
IRYDMAGC

SEQ ID NO: 59 - protein, 8 aa, Artificial Sequence
  Description of Artificial Sequence Synthetic mimotope peptide
IRWDTSLC

SEQ ID NO: 60 - protein, 7 aa, Artificial Sequence
  Description of Artificial Sequence Synthetic mimotope peptide
IRWDQPC

SEQ ID NO: 61 - protein, 6 aa, Artificial Sequence
  Description of Artificial Sequence Synthetic mimotope peptide
IRWDGC

SEQ ID NO: 62 - protein, 7 aa, Artificial Sequence
  Description of Artificial Sequence Synthetic mimotope peptide
IRWDGGC

SEQ ID NO: 63 - protein, 8 aa, Artificial Sequence
  Description of Artificial Sequence Synthetic mimotope peptide
EVWHRHQC

SEQ ID NO: 64 - protein, 8 aa, Artificial Sequence
  Description of Artificial Sequence Synthetic mimotope peptide
ERWHEKHC

SEQ ID NO: 65 - protein, 8 aa, Artificial Sequence
  Description of Artificial Sequence Synthetic mimotope peptide
EVWHRLQC

SEQ ID NO: 66 - protein, 8 aa, Artificial Sequence
  Description of Artificial Sequence Synthetic mimotope peptide
ELWHRYPC

SEQ ID NO: 67 - protein, 8 aa, Artificial Sequence
  Description of Artificial Sequence Synthetic mimotope peptide
ELWHRAFC

SEQ ID NO: 68 - protein, 7 aa, Artificial Sequence
  Description of Artificial Sequence Synthetic mimotope peptide
ELWHRAC

SEQ ID NO: 69 - protein, 7 aa, Artificial Sequence
  Description of Artificial Sequence Synthetic mimotope peptide
EVWHRGC

SEQ ID NO: 70 - protein, 7 aa, Artificial Sequence
  Description of Artificial Sequence Synthetic mimotope peptide
EVWHRHC

SEQ ID NO: 71 - protein, 7 aa, Artificial Sequence
  Description of Artificial Sequence Synthetic mimotope peptide
ERWHEKC

SEQ ID NO: 72 - protein, 7 aa, Artificial Sequence
  Description of Artificial Sequence Synthetic mimotope peptide
QDFRHYC

SEQ ID NO: 73 - protein, 7 aa, Artificial Sequence
  Description of Artificial Sequence Synthetic mimotope peptide
SEFKHGC

SEQ ID NO: 74 - protein, 7 aa, Artificial Sequence
  Description of Artificial Sequence Synthetic mimotope peptide
TSFRHGC

SEQ ID NO: 75 - protein, 7 aa, Artificial Sequence
  Description of Artificial Sequence Synthetic mimotope peptide
TSVFRHC

SEQ ID NO: 76 - protein, 7 aa, Artificial Sequence
  Description of Artificial Sequence Synthetic mimotope peptide
TPFRHTC

SEQ ID NO: 77 - protein, 7 aa, Artificial Sequence
  Description of Artificial Sequence Synthetic mimotope peptide
SQFRHYC

SEQ ID NO: 78 - protein, 7 aa, Artificial Sequence
  Description of Artificial Sequence Synthetic mimotope peptide
LMFRHNC

SEQ ID NO: 79 - protein, 7 aa, Artificial Sequence
  Description of Artificial Sequence Synthetic mimotope peptide
SAFRHHC

SEQ ID NO: 80 - protein, 7 aa, Artificial Sequence
  Description of Artificial Sequence Synthetic mimotope peptide
LPFRHGC

SEQ ID NO: 81 - protein, 7 aa, Artificial Sequence
  Description of Artificial Sequence Synthetic mimotope peptide
SHFRHGC

SEQ ID NO: 82 - protein, 7 aa, Artificial Sequence
  Description of Artificial Sequence Synthetic mimotope peptide
ILFRHGC

SEQ ID NO: 83 - protein, 7 aa, Artificial Sequence
  Description of Artificial Sequence Synthetic mimotope peptide
QFKHDLC

SEQ ID NO: 84 - protein, 7 aa, Artificial Sequence
  Description of Artificial Sequence Synthetic mimotope peptide
NWFPHPC

SEQ ID NO: 85 - protein, 7 aa, Artificial Sequence
  Description of Artificial Sequence Synthetic mimotope peptide
EEFKYSC

SEQ ID NO: 86 - protein, 8 aa, Artificial Sequence
  Description of Artificial Sequence Synthetic mimotope peptide
NELRHSTC

SEQ ID NO: 87 - protein, 8 aa, Artificial Sequence
  Description of Artificial Sequence Synthetic mimotope peptide
GEMRHQPC

SEQ ID NO: 88 - protein, 8 aa, Artificial Sequence
  Description of Artificial Sequence Synthetic mimotope peptide
DTYFPRSC

SEQ ID NO: 89 - protein, 8 aa, Artificial Sequence
  Description of Artificial Sequence Synthetic mimotope peptide
VELRHSRC

SEQ ID NO: 90 - protein, 8 aa, Artificial Sequence
  Description of Artificial Sequence Synthetic mimotope peptide
YSMRHDAC

SEQ ID NO: 91 - protein, 8 aa, Artificial Sequence
  Description of Artificial Sequence Synthetic mimotope peptide
AANYFPRC

SEQ ID NO: 92 - protein, 8 aa, Artificial Sequence
  Description of Artificial Sequence Synthetic mimotope peptide
SPNQFRHC

SEQ ID NO: 93 - protein, 8 aa, Artificial Sequence
  Description of Artificial Sequence Synthetic mimotope peptide
SSSFFPRC

SEQ ID NO: 94 - protein, 8 aa, Artificial Sequence
  Description of Artificial Sequence Synthetic mimotope peptide
EDWFFWHC

SEQ ID NO: 95 - protein, 8 aa, Artificial Sequence
  Description of Artificial Sequence Synthetic mimotope peptide
SAGSFRHC

SEQ ID NO: 96 - protein, 8 aa, Artificial Sequence
  Description of Artificial Sequence Synthetic mimotope peptide
QVMRHHAC

SEQ ID NO: 97 - protein, 8 aa, Artificial Sequence
  Description of Artificial Sequence Synthetic mimotope peptide
SEFSHSSC

SEQ ID NO: 98 - protein, 8 aa, Artificial Sequence
  Description of Artificial Sequence Synthetic mimotope peptide
QPNLFYHC

SEQ ID NO: 99 - protein, 8 aa, Artificial Sequence
  Description of Artificial Sequence Synthetic mimotope peptide
ELFKHHLC

SEQ ID NO: 100 - protein, 8 aa, Artificial Sequence
  Description of Artificial Sequence Synthetic mimotope peptide
TLHEFRHC

SEQ ID NO: 101 - protein, 8 aa, Artificial Sequence
  Description of Artificial Sequence Synthetic mimotope peptide
ATFRHSPC

SEQ ID NO: 102 - protein, 8 aa, Artificial Sequence
  Description of Artificial Sequence Synthetic mimotope peptide
APMYFPHC

SEQ ID NO: 103 - protein, 8 aa, Artificial Sequence
  Description of Artificial Sequence Synthetic mimotope peptide
TYFSHSLC

SEQ ID NO: 104 - protein, 8 aa, Artificial Sequence
  Description of Artificial Sequence Synthetic mimotope peptide
HEPLFSHC

SEQ ID NO: 105 - protein, 8 aa, Artificial Sequence
  Description of Artificial Sequence Synthetic mimotope peptide
SLMRHSSC

SEQ ID NO: 106 - protein, 8 aa, Artificial Sequence
  Description of Artificial Sequence Synthetic mimotope peptide
EFLRHTLC

SEQ ID NO: 107 - protein, 8 aa, Artificial Sequence
  Description of Artificial Sequence Synthetic mimotope peptide
ATPLFRHC

SEQ ID NO: 108 - protein, 8 aa, Artificial Sequence
  Description of Artificial Sequence Synthetic mimotope peptide
QELKRYYC

SEQ ID NO: 109 - protein, 8 aa, Artificial Sequence
  Description of Artificial Sequence Synthetic mimotope peptide
THTDFRHC

SEQ ID NO: 110 - protein, 8 aa, Artificial Sequence
  Description of Artificial Sequence Synthetic mimotope peptide
LHIPFRHC

SEQ ID NO: 111 - protein, 8 aa, Artificial Sequence
  Description of Artificial Sequence Synthetic mimotope peptide
NELFKHFC

SEQ ID NO: 112 - protein, 8 aa, Artificial Sequence
  Description of Artificial Sequence Synthetic mimotope peptide
SQYFPRPC

SEQ ID NO: 113 - protein, 8 aa, Artificial Sequence
  Description of Artificial Sequence Synthetic mimotope peptide
DEHPFRHC

SEQ ID NO: 114 - protein, 8 aa, Artificial Sequence
  Description of Artificial Sequence Synthetic mimotope peptide
MLPFRHGC

SEQ ID NO: 115 - protein, 8 aa, Artificial Sequence
  Description of Artificial Sequence Synthetic mimotope peptide
SAMRHSLC

SEQ ID NO: 116 - protein, 8 aa, Artificial Sequence
  Description of Artificial Sequence Synthetic mimotope peptide
TPLMFWHC

SEQ ID NO: 117 - protein, 8 aa, Artificial Sequence
  Description of Artificial Sequence Synthetic mimotope peptide
LQFKHSTC

SEQ ID NO: 118 - protein, 8 aa, Artificial Sequence
  Description of Artificial Sequence Synthetic mimotope peptide
ATFRHSTC

SEQ ID NO: 119 - protein, 8 aa, Artificial Sequence
  Description of Artificial Sequence Synthetic mimotope peptide
TGLMFKHC

SEQ ID NO: 120 - protein, 8 aa, Artificial Sequence
  Description of Artificial Sequence Synthetic mimotope peptide
AEFSHWHC

SEQ ID NO: 121 - protein, 8 aa, Artificial Sequence
  Description of Artificial Sequence Synthetic mimotope peptide
QSEFKHWC

SEQ ID NO: 122 - protein, 8 aa, Artificial Sequence
  Description of Artificial Sequence Synthetic mimotope peptide
AEFMHSVC

SEQ ID NO: 123 - protein, 8 aa, Artificial Sequence
  Description of Artificial Sequence Synthetic mimotope peptide
ADHDFRHC

SEQ ID NO: 124 - protein, 8 aa, Artificial Sequence
  Description of Artificial Sequence Synthetic mimotope peptide
DGLLFKHC

SEQ ID NO: 125 - protein, 8 aa, Artificial Sequence
  Description of Artificial Sequence Synthetic mimotope peptide
IGFRHDSC

SEQ ID NO: 126 - protein, 8 aa, Artificial Sequence
  Description of Artificial Sequence Synthetic mimotope peptide
SNSEFRRC

SEQ ID NO: 127 - protein, 8 aa, Artificial Sequence
  Description of Artificial Sequence Synthetic mimotope peptide
SELRHSTC

SEQ ID NO: 128 - protein, 8 aa, Artificial Sequence
  Description of Artificial Sequence Synthetic mimotope peptide
THMEFRRC

SEQ ID NO: 129 - protein, 8 aa, Artificial Sequence
  Description of Artificial Sequence Synthetic mimotope peptide
EELRHSVC

SEQ ID NO: 130 - protein, 8 aa, Artificial Sequence
  Description of Artificial Sequence Synthetic mimotope peptide
QLFKHSPC

SEQ ID NO: 131 - protein, 8 aa, Artificial Sequence
  Description of Artificial Sequence Synthetic mimotope peptide
YEFRHAQC

SEQ ID NO: 132 - protein, 8 aa, Artificial Sequence
  Description of Artificial Sequence Synthetic mimotope peptide
SNFRHSVC

SEQ ID NO: 133 - protein, 8 aa, Artificial Sequence
  Description of Artificial Sequence Synthetic mimotope peptide
APIQFRHC

SEQ ID NO: 134 - protein, 8 aa, Artificial Sequence
  Description of Artificial Sequence Synthetic mimotope peptide
AYFPHTSC

SEQ ID NO: 135 - protein, 8 aa, Artificial Sequence
  Description of Artificial Sequence Synthetic mimotope peptide
NSSELRHC

SEQ ID NO: 136 - protein, 8 aa, Artificial Sequence
  Description of Artificial Sequence Synthetic mimotope peptide
TEFRHKAC

SEQ ID NO: 137 - protein, 8 aa, Artificial Sequence
  Description of Artificial Sequence Synthetic mimotope peptide
TSTEMWHC

SEQ ID NO: 138 - protein, 8 aa, Artificial Sequence
  Description of Artificial Sequence Synthetic mimotope peptide
SQSYFKHC

SEQ ID NO: 139 - protein, 6 aa, Artificial Sequence
  Description of Artificial Sequence Synthetic mimotope peptide
CSEFKH

SEQ ID NO: 140 - protein, 6 aa, Artificial Sequence
  Description of Artificial Sequence Synthetic mimotope peptide
SEFKHC

SEQ ID NO: 141 - protein, 6 aa, Artificial Sequence
  Description of Artificial Sequence Synthetic mimotope peptide
CHEFRH

SEQ ID NO: 142 - protein, 6 aa, Artificial Sequence
  Description of Artificial Sequence Synthetic mimotope peptide
HEFRHC

SEQ ID NO: 143 - protein, 11 aa, Artificial Sequence
  Description of Artificial Sequence Synthetic mimotope peptide
DAEFRHDSGYC

SEQ ID NO: 144 - protein, 7 aa, Artificial Sequence
  Description of Artificial Sequence Synthetic mimotope peptide
EFRHDSC

SEQ ID NO: 145 - protein, 7 aa, Artificial Sequence
  Description of Artificial Sequence Synthetic mimotope peptide
EVHHQKC

SEQ ID NO: 146 - protein, 7 aa, Artificial Sequence
  Description of Artificial Sequence Synthetic mimotope peptide
EFRHDSC

SEQ ID NO: 147 - protein, 10 aa, Artificial Sequence
  Description of Artificial Sequence Synthetic mimotope peptide
EVHHQKLVFC

SEQ ID NO: 148 - protein, 9 aa, Artificial Sequence
  Description of Artificial Sequence Synthetic mimotope peptide
GYEVHHQKC

SEQ ID NO: 149 - protein, 10 aa, Artificial Sequence
  Description of Artificial Sequence Synthetic mimotope peptide
EVHHQKLVFC

SEQ ID NO: 150 - protein, 11 aa, Artificial Sequence
  Description of Artificial Sequence Synthetic mimotope peptide
CEVHHQKLVFF

SEQ ID NO: 151 - protein, 9 aa, Artificial Sequence
  Description of Artificial Sequence Synthetic mimotope peptide
CGLMVGGVV

SEQ ID NO: 152 - protein, 40 aa, Artificial Sequence
  Description of Artificial Sequence Synthetic mimotope polypeptide
DAEFRHDSGYEVHHQKLVFFAEDVGSNKGAIIGLMVGGVV

SEQ ID NO: 153 - protein, 42 aa, Artificial Sequence [sequence omitted]

SEQ ID NO: 154 - protein, 8 aa, Artificial Sequence
  Description of Artificial Sequence Synthetic mimotope peptide
XRXDXXXC

SEQ ID NO: 155 - protein, 8 aa, Artificial Sequence
  Description of Artificial Sequence Synthetic mimotope peptide
EXWHXXXC

SEQ ID NO: 156 - protein, 8 aa, Artificial Sequence
  Description of Artificial Sequence Synthetic amyloid-beta-peptide fragment
EFRHDSGY

SEQ ID NO: 157 - protein, 8 aa, Artificial Sequence
  Description of Artificial Sequence Synthetic amyloid-beta-peptide fragment
EFRHDSGY

SEQ ID NO: 158 - protein, 7 aa, Artificial Sequence
  Description of Artificial Sequence Synthetic amyloid-beta-peptide fragment
EVHHQKL

SEQ ID NO: 159 - protein, 8 aa, Artificial Sequence
  Description of Artificial Sequence Synthetic mimotope peptide
SHTRLYFC

SEQ ID NO: 160 - protein, 8 aa, Artificial Sequence
  Description of Artificial Sequence Synthetic mimotope peptide
SGEYVFHC

SEQ ID NO: 161 - protein, 8 aa, Artificial Sequence
  Description of Artificial Sequence Synthetic mimotope peptide
SGQLKFPC

SEQ ID NO: 162 - protein, 8 aa, Artificial Sequence
  Description of Artificial Sequence Synthetic mimotope peptide
SGQIWFRC

SEQ ID NO: 163 - protein, 8 aa, Artificial Sequence
  Description of Artificial Sequence Synthetic mimotope peptide
SGEIHFNC

SEQ ID NO: 164 - protein, 8 aa, Artificial Sequence
  Description of Artificial Sequence Synthetic mimotope peptide
GQIWFISC

SEQ ID NO: 165 - protein, 8 aa, Artificial Sequence
  Description of Artificial Sequence Synthetic mimotope peptide
NDAKIVFC

SEQ ID NO: 166 - protein, 8 aa, Artificial Sequence
  Description of Artificial Sequence Synthetic mimotope peptide
GQIIFQSC

SEQ ID NO: 167 - protein, 8 aa, Artificial Sequence
  Description of Artificial Sequence Synthetic mimotope peptide
GQIRFDHC

SEQ ID NO: 168 - protein, 8 aa, Artificial Sequence
  Description of Artificial Sequence Synthetic mimotope peptide
HMRLFFNC

SEQ ID NO: 169 - protein, 8 aa, Artificial Sequence
  Description of Artificial Sequence Synthetic mimotope peptide
GEMWFALC

SEQ ID NO: 170 - protein, 8 aa, Artificial Sequence
  Description of Artificial Sequence Synthetic mimotope peptide
GELQFPPC

SEQ ID NO: 171 - protein, 7 aa, Artificial Sequence
  Description of Artificial Sequence Synthetic mimotope peptide
GELWFPC

SEQ ID NO: 172 - protein, 8 aa, Artificial Sequence
  Description of Artificial Sequence Synthetic mimotope peptide
SHQRLWFC

SEQ ID NO: 173 - protein, 8 aa, Artificial Sequence
  Description of Artificial Sequence Synthetic mimotope peptide
HQKMIFAC

SEQ ID NO: 174 - protein, 8 aa, Artificial Sequence
  Description of Artificial Sequence Synthetic mimotope peptide
GEMQFFIC

SEQ ID NO: 175 - protein, 8 aa, Artificial Sequence
  Description of Artificial Sequence Synthetic mimotope peptide
GELYFRAC

SEQ ID NO: 176 - protein, 8 aa, Artificial Sequence
  Description of Artificial Sequence Synthetic mimotope peptide
GEIRFALC

SEQ ID NO: 177 - protein, 8 aa, Artificial Sequence
  Description of Artificial Sequence Synthetic mimotope peptide
GMIVFPHC

SEQ ID NO: 178 - protein, 8 aa, Artificial Sequence
  Description of Artificial Sequence Synthetic mimotope peptide
GEIWFEGC

SEQ ID NO: 179 - protein, 8 aa, Artificial Sequence
  Description of Artificial Sequence Synthetic mimotope peptide
GEIYFERC

SEQ ID NO: 180 - protein, 8 aa, Artificial Sequence
  Description of Artificial Sequence Synthetic mimotope peptide
AIPLFVMC

SEQ ID NO: 181 - protein, 8 aa, Artificial Sequence
  Description of Artificial Sequence Synthetic mimotope peptide
GDLKFPLC

SEQ ID NO: 182 - protein, 8 aa, Artificial Sequence
  Description of Artificial Sequence Synthetic mimotope peptide
GQILFPVC

SEQ ID NO: 183 - protein, 8 aa, Artificial Sequence
  Description of Artificial Sequence Synthetic mimotope peptide
GELFFPKC

SEQ ID NO: 184 - protein, 8 aa, Artificial Sequence
  Description of Artificial Sequence Synthetic mimotope peptide
GQIMFPRC

SEQ ID NO: 185 - protein, 8 aa, Artificial Sequence
  Description of Artificial Sequence Synthetic mimotope peptide
HMRMYFEC

SEQ ID NO: 186 - protein, 8 aa, Artificial Sequence
  Description of Artificial Sequence Synthetic mimotope peptide
GSLFFWPC

SEQ ID NO: 187 - protein, 8 aa, Artificial Sequence
  Description of Artificial Sequence Synthetic mimotope peptide
GEILFGMC

SEQ ID NO: 188 - protein, 8 aa, Artificial Sequence
  Description of Artificial Sequence Synthetic mimotope peptide
GQLKFPFC

SEQ ID NO: 189 - protein, 8 aa, Artificial Sequence
  Description of Artificial Sequence Synthetic mimotope peptide
KLPLFVMC

SEQ ID NO: 190 - protein, 8 aa, Artificial Sequence
  Description of Artificial Sequence Synthetic mimotope peptide
GTIFFRDC

SEQ ID NO: 191 - protein, 8 aa, Artificial Sequence
  Description of Artificial Sequence Synthetic mimotope peptide
THQRLWFC

SEQ ID NO: 192 - protein, 8 aa, Artificial Sequence
  Description of Artificial Sequence Synthetic mimotope peptide
GQIKFAQC

SEQ ID NO: 193 - protein, 8 aa, Artificial Sequence
  Description of Artificial Sequence Synthetic mimotope peptide
GTLIFHHC

SEQ ID NO: 194 - protein, 8 aa, Artificial Sequence
  Description of Artificial Sequence Synthetic mimotope peptide
GEIRFGSC

SEQ ID NO: 195 - protein, 8 aa, Artificial Sequence
  Description of Artificial Sequence Synthetic mimotope peptide
GQIQFPLC

SEQ ID NO: 196 - protein, 8 aa, Artificial Sequence
  Description of Artificial Sequence Synthetic mimotope peptide
GEIKFDHC

SEQ ID NO: 197 - protein, 8 aa, Artificial Sequence
  Description of Artificial Sequence Synthetic mimotope peptide
GEIQFGAC

SEQ ID NO: 198 - protein, 8 aa, Artificial Sequence
  Description of Artificial Sequence Synthetic mimotope peptide
QLPLFVLC

SEQ ID NO: 199 - protein, 7 aa, Artificial Sequence
  Description of Artificial Sequence Synthetic mimotope peptide
HQKMIFC

SEQ ID NO: 200 - protein, 8 aa, Artificial Sequence
  Description of Artificial Sequence Synthetic mimotope peptide
GELFFEKC

SEQ ID NO: 201 - protein, 8 aa, Artificial Sequence
  Description of Artificial Sequence Synthetic mimotope peptide
GEIRFELC

SEQ ID NO: 202 - protein, 8 aa, Artificial Sequence
  Description of Artificial Sequence Synthetic mimotope peptide
GEIYFERC

SEQ ID NO: 203 - protein, 9 aa, Artificial Sequence
  Description of Artificial Sequence Synthetic mimotope peptide
SGEIYFERC

SEQ ID NO: 204 - protein, 9 aa, Artificial Sequence
  Description of Artificial Sequence Synthetic mimotope peptide
AGEIYFERC

SEQ ID NO: 205 - protein, 8 aa, Artificial Sequence
  Description of Artificial Sequence Synthetic mimotope peptide
CGEIYFER

SEQ ID NO: 206 - protein, 11 aa, Artificial Sequence
  Description of Artificial Sequence Synthetic mimotope peptide
DAEFRHDSGYC

SEQ ID NO: 207 - protein, 11 aa, Artificial Sequence
  Description of Artificial Sequence Synthetic mimotope peptide
CHQKLVFFAED

SEQ ID NO: 208 - protein, 10 aa, Artificial Sequence
  Description of Artificial Sequence Synthetic mimotope peptide
EVHHQKLVFC

SEQ ID NO: 209 - protein, 10 aa, Artificial Sequence
  Description of Artificial Sequence Synthetic mimotope peptide
EVHHQKLVFC

SEQ ID NO: 210 - protein, 9 aa, Artificial Sequence
  Description of Artificial Sequence Synthetic mimotope peptide
CGLMVGGVV

SEQ ID NO: 211 - protein, 40 aa, Artificial Sequence; identical to SEQ ID NO: 152
  Description of Artificial Sequence Synthetic mimotope polypeptide

SEQ ID NO: 212 - protein, 42 aa, Artificial Sequence [sequence omitted]

SEQ ID NO: 213 - protein, 9 aa, Artificial Sequence
  Description of Artificial Sequence Synthetic mimotope peptide
XGXXXFXXC

SEQ ID NO: 214 - protein, 10 aa, Artificial Sequence
  Description of Artificial Sequence Synthetic mimotope peptide
XMHXXXXFCX

SEQ ID NO: 215 - protein, 6 aa, Artificial Sequence
  Description of Artificial Sequence Synthetic amyloid-beta-peptide fragment
HQKLVF

SEQ ID NO: 216 - protein, 10 aa, Artificial Sequence
  Description of Artificial Sequence Synthetic amyloid-beta-peptide fragment
HQKLVFFAED

SEQ ID NO: 217 - protein, 7 aa, Artificial Sequence
  Description of Artificial Sequence Synthetic peptide
XXXXXXX

SEQ ID NO: 218 - protein, 6 aa, Artificial Sequence
  Description of Artificial Sequence Synthetic peptide
YREFFI

SEQ ID NO: 219 - protein, 7 aa, Artificial Sequence
  Description of Artificial Sequence Synthetic peptide
IRWDTPC

SEQ ID NO: 220 - protein, 8 aa, Artificial Sequence
  Description of Artificial Sequence Synthetic peptide
VRWDVYPC

SEQ ID NO: 221 - protein, 8 aa, Artificial Sequence
  Description of Artificial Sequence Synthetic peptide
IRYDAPLC

SEQ ID NO: 222 - protein, 8 aa, Artificial Sequence
  Description of Artificial Sequence Synthetic peptide
IRYDMAGC

SEQ ID NO: 223 - protein, 8 aa, Artificial Sequence
  Description of Artificial Sequence Synthetic peptide
IRWDTSLC

SEQ ID NO: 224 - protein, 7 aa, Artificial Sequence
  Description of Artificial Sequence Synthetic peptide
IRWDQPC

SEQ ID NO: 225 - protein, 6 aa, Artificial Sequence
  Description of Artificial Sequence Synthetic peptide
IRWDGC

SEQ ID NO: 226 - protein, 7 aa, Artificial Sequence
  Description of Artificial Sequence Synthetic peptide
IRWDGGC

SEQ ID NO: 227 - protein, 8 aa, Artificial Sequence
  Description of Artificial Sequence Synthetic peptide
EVWHRHQC

SEQ ID NO: 228 - protein, 8 aa, Artificial Sequence
  Description of Artificial Sequence Synthetic peptide
ERWHEKHC

SEQ ID NO: 229 - protein, 8 aa, Artificial Sequence
  Description of Artificial Sequence Synthetic peptide
EVWHRLQC

SEQ ID NO: 230 - protein, 8 aa, Artificial Sequence
  Description of Artificial Sequence Synthetic peptide
ELWHRYPC

SEQ ID NO: 231 - protein, 8 aa, Artificial Sequence
  Description of Artificial Sequence Synthetic peptide
ELWHRAFC

SEQ ID NO: 232 - protein, 7 aa, Artificial Sequence
  Description of Artificial Sequence Synthetic peptide
ELWHRAC

SEQ ID NO: 233 - protein, 7 aa, Artificial Sequence
  Description of Artificial Sequence Synthetic peptide
EVWHRGC

SEQ ID NO: 234 - protein, 7 aa, Artificial Sequence
  Description of Artificial Sequence Synthetic peptide
EVWHRHC

SEQ ID NO: 235 - protein, 7 aa, Artificial Sequence
  Description of Artificial Sequence Synthetic peptide
ERWHEKC

SEQ ID NO: 236 - protein, 7 aa, Artificial Sequence
  Description of Artificial Sequence Synthetic peptide
QDFRHYC

SEQ ID NO: 237 - protein, 7 aa, Artificial Sequence
  Description of Artificial Sequence Synthetic peptide
SEFKHGC

SEQ ID NO: 238 - protein, 7 aa, Artificial Sequence
  Description of Artificial Sequence Synthetic peptide
TSFRHGC

SEQ ID NO: 239 - protein, 7 aa, Artificial Sequence
  Description of Artificial Sequence Synthetic peptide
TSVFRHC

SEQ ID NO: 240 - protein, 7 aa, Artificial Sequence
  Description of Artificial Sequence Synthetic peptide
TPFRHTC

SEQ ID NO: 241 - protein, 7 aa, Artificial Sequence
  Description of Artificial Sequence Synthetic peptide
SQFRHYC

SEQ ID NO: 242 - protein, 7 aa, Artificial Sequence
  Description of Artificial Sequence Synthetic peptide
LMFRHNC

SEQ ID NO: 243 - protein, 7 aa, Artificial Sequence
  Description of Artificial Sequence Synthetic peptide
SAFRHHC

SEQ ID NO: 244 - protein, 7 aa, Artificial Sequence
  Description of Artificial Sequence Synthetic peptide
LPFRHGC

SEQ ID NO: 245 - protein, 7 aa, Artificial Sequence
  Description of Artificial Sequence Synthetic peptide
SHFRHGC

SEQ ID NO: 246 - protein, 7 aa, Artificial Sequence
  Description of Artificial Sequence Synthetic peptide
ILFRHGC

SEQ ID NO: 247 - protein, 7 aa, Artificial Sequence
  Description of Artificial Sequence Synthetic peptide
QFKHDLC

SEQ ID NO: 248 - protein, 7 aa, Artificial Sequence
  Description of Artificial Sequence Synthetic peptide
NWFPHPC

SEQ ID NO: 249 - protein, 7 aa, Artificial Sequence
  Description of Artificial Sequence Synthetic peptide
EEFKYSC

SEQ ID NO: 250 - protein, 8 aa, Artificial Sequence
  Description of Artificial Sequence Synthetic peptide
NELRHSTC

SEQ ID NO: 251 - protein, 8 aa, Artificial Sequence
  Description of Artificial Sequence Synthetic peptide
GEMRHQPC

SEQ ID NO: 252 - protein, 8 aa, Artificial Sequence
  Description of Artificial Sequence Synthetic peptide
DTYFPRSC

SEQ ID NO: 253 - protein, 8 aa, Artificial Sequence
  Description of Artificial Sequence Synthetic peptide
VELRHSRC

SEQ ID NO: 254 - protein, 8 aa, Artificial Sequence
  Description of Artificial Sequence Synthetic peptide
YSMRHDAC

SEQ ID NO: 255 - protein, 8 aa, Artificial Sequence
  Description of Artificial Sequence Synthetic peptide
AANYFPRC

SEQ ID NO: 256 - protein, 8 aa, Artificial Sequence
  Description of Artificial Sequence Synthetic peptide
SPNQFRHC

SEQ ID NO: 257 - protein, 8 aa, Artificial Sequence
  Description of Artificial Sequence Synthetic peptide
SSSFFPRC

SEQ ID NO: 258 - protein, 8 aa, Artificial Sequence
  Description of Artificial Sequence Synthetic peptide
EDWFFWHC

SEQ ID NO: 259 - protein, 8 aa, Artificial Sequence
  Description of Artificial Sequence Synthetic peptide
SAGSFRHC

SEQ ID NO: 260 - protein, 8 aa, Artificial Sequence
  Description of Artificial Sequence Synthetic peptide
QVMRHHAC

SEQ ID NO: 261 - protein, 8 aa, Artificial Sequence
  Description of Artificial Sequence Synthetic peptide
SEFSHSSC

SEQ ID NO: 262 - protein, 8 aa, Artificial Sequence
  Description of Artificial Sequence Synthetic peptide
QPNLFYHC

SEQ ID NO: 263 - protein, 8 aa, Artificial Sequence
  Description of Artificial Sequence Synthetic peptide
ELFKHHLC

SEQ ID NO: 264 - protein, 8 aa, Artificial Sequence
  Description of Artificial Sequence Synthetic peptide
TLHEFRHC

SEQ ID NO: 265 - protein, 8 aa, Artificial Sequence
  Description of Artificial Sequence Synthetic peptide
ATFRHSPC

SEQ ID NO: 266 - protein, 8 aa, Artificial Sequence
  Description of Artificial Sequence Synthetic peptide
APMYFPHC

SEQ ID NO: 267 - protein, 8 aa, Artificial Sequence
  Description of Artificial Sequence Synthetic peptide
TYFSHSLC

SEQ ID NO: 268 - protein, 8 aa, Artificial Sequence
  Description of Artificial Sequence Synthetic peptide
HEPLFSHC

SEQ ID NO: 269 - protein, 8 aa, Artificial Sequence
  Description of Artificial Sequence Synthetic peptide
SLMRHSSC

SEQ ID NO: 270 - protein, 8 aa, Artificial Sequence
  Description of Artificial Sequence Synthetic peptide
EFLRHTLC

SEQ ID NO: 271 - protein, 8 aa, Artificial Sequence
  Description of Artificial Sequence Synthetic peptide
ATPLFRHC

SEQ ID NO: 272 - protein, 8 aa, Artificial Sequence
  Description of Artificial Sequence Synthetic peptide
QELKRYYC

SEQ ID NO: 273 - protein, 8 aa, Artificial Sequence
  Description of Artificial Sequence Synthetic peptide
THTDFRHC

SEQ ID NO: 274 - protein, 8 aa, Artificial Sequence
  Description of Artificial Sequence Synthetic peptide
LHIPFRHC

SEQ ID NO: 275 - protein, 8 aa, Artificial Sequence
  Description of Artificial Sequence Synthetic peptide
NELFKHFC

SEQ ID NO: 276 - protein, 8 aa, Artificial Sequence
  Description of Artificial Sequence Synthetic peptide
SQYFPRPC

SEQ ID NO: 277 - protein, 8 aa, Artificial Sequence
  Description of Artificial Sequence Synthetic peptide
DEHPFRHC

SEQ ID NO: 278 - protein, 8 aa, Artificial Sequence
  Description of Artificial Sequence Synthetic peptide
MLPFRHGC

SEQ ID NO: 279 - protein, 8 aa, Artificial Sequence
  Description of Artificial Sequence Synthetic peptide
SAMRHSLC

SEQ ID NO: 280 - protein, 8 aa, Artificial Sequence
  Description of Artificial Sequence Synthetic peptide
TPLMFWHC

SEQ ID NO: 281 - protein, 8 aa, Artificial Sequence
  Description of Artificial Sequence Synthetic peptide
LQFKHSTC

SEQ ID NO: 282 - protein, 8 aa, Artificial Sequence
  Description of Artificial Sequence Synthetic peptide
ATFRHSTC

SEQ ID NO: 283 - protein, 8 aa, Artificial Sequence
  Description of Artificial Sequence Synthetic peptide
TGLMFKHC

SEQ ID NO: 284 - protein, 8 aa, Artificial Sequence
  Description of Artificial Sequence Synthetic peptide
AEFSHWHC

SEQ ID NO: 285 - protein, 8 aa, Artificial Sequence
  Description of Artificial Sequence Synthetic peptide
QSEFKHWC

SEQ ID NO: 286 - protein, 8 aa, Artificial Sequence
  Description of Artificial Sequence Synthetic peptide
AEFMHSVC

SEQ ID NO: 287 - protein, 8 aa, Artificial Sequence
  Description of Artificial Sequence Synthetic peptide
ADHDFRHC

SEQ ID NO: 288 - protein, 8 aa, Artificial Sequence
  Description of Artificial Sequence Synthetic peptide
DGLLFKHC

SEQ ID NO: 289 - protein, 8 aa, Artificial Sequence
  Description of Artificial Sequence Synthetic peptide
IGFRHDSC

SEQ ID NO: 290 - protein, 8 aa, Artificial Sequence
  Description of Artificial Sequence Synthetic peptide
SNSEFRRC

SEQ ID NO: 291 - protein, 8 aa, Artificial Sequence
  Description of Artificial Sequence Synthetic peptide
SELRHSTC

SEQ ID NO: 292 - protein, 8 aa, Artificial Sequence
  Description of Artificial Sequence Synthetic peptide
THMEFRRC

SEQ ID NO: 293 - protein, 8 aa, Artificial Sequence
  Description of Artificial Sequence Synthetic peptide
EELRHSVC

SEQ ID NO: 294 - protein, 8 aa, Artificial Sequence
  Description of Artificial Sequence Synthetic peptide
QLFKHSPC

SEQ ID NO: 295 - protein, 8 aa, Artificial Sequence
  Description of Artificial Sequence Synthetic peptide
YEFRHAQC

SEQ ID NO: 296 - protein, 8 aa, Artificial Sequence
  Description of Artificial Sequence Synthetic peptide
SNFRHSVC

SEQ ID NO: 297 - protein, 8 aa, Artificial Sequence
  Description of Artificial Sequence Synthetic peptide
APIQFRHC

SEQ ID NO: 298 - protein, 8 aa, Artificial Sequence
  Description of Artificial Sequence Synthetic peptide
AYFPHTSC

SEQ ID NO: 299 - protein, 8 aa, Artificial Sequence
  Description of Artificial Sequence Synthetic peptide
NSSELRHC

SEQ ID NO: 300 - protein, 8 aa, Artificial Sequence
  Description of Artificial Sequence Synthetic peptide
TEFRHKAC

SEQ ID NO: 301 - protein, 8 aa, Artificial Sequence
  Description of Artificial Sequence Synthetic peptide
TSTEMWHC

SEQ ID NO: 302 - protein, 8 aa, Artificial Sequence
  Description of Artificial Sequence Synthetic peptide
SQSYFKHC

SEQ ID NO: 303 - protein, 6 aa, Artificial Sequence
  Description of Artificial Sequence Synthetic peptide
CSEFKH

SEQ ID NO: 304 - protein, 6 aa, Artificial Sequence
  Description of Artificial Sequence Synthetic peptide
SEFKHC

SEQ ID NO: 305 - protein, 6 aa, Artificial Sequence
  Description of Artificial Sequence Synthetic peptide
CHEFRH

SEQ ID NO: 306 - protein, 6 aa, Artificial Sequence
  Description of Artificial Sequence Synthetic peptide
HEFRHC

SEQ ID NO: 307 - protein, 8 aa, Artificial Sequence
  Description of Artificial Sequence Synthetic peptide
SGEYVFHC

SEQ ID NO: 308 - protein, 8 aa, Artificial Sequence
  Description of Artificial Sequence Synthetic peptide
SGQLKFPC

SEQ ID NO: 309 - protein, 8 aa, Artificial Sequence
  Description of Artificial Sequence Synthetic peptide
SGQIWFRC

SEQ ID NO: 310 - protein, 8 aa, Artificial Sequence
  Description of Artificial Sequence Synthetic peptide
SGEIHFNC

SEQ ID NO: 311 - protein, 8 aa, Artificial Sequence
  Description of Artificial Sequence Synthetic peptide
GQIWFISC

SEQ ID NO: 312 - protein, 8 aa, Artificial Sequence
  Description of Artificial Sequence Synthetic peptide
GQIIFQSC

SEQ ID NO: 313 - protein, 8 aa, Artificial Sequence
  Description of Artificial Sequence Synthetic peptide
GQIRFDHC

SEQ ID NO: 314 - protein, 8 aa, Artificial Sequence
  Description of Artificial Sequence Synthetic peptide
GEMWFALC

SEQ ID NO: 315 - protein, 8 aa, Artificial Sequence
  Description of Artificial Sequence Synthetic peptide
GELQFPPC

SEQ ID NO: 316 - protein, 7 aa, Artificial Sequence
  Description of Artificial Sequence Synthetic peptide
GELWFPC

SEQ ID NO: 317 - protein, 8 aa, Artificial Sequence
  Description of Artificial Sequence Synthetic peptide
GEMQFFIC

SEQ ID NO: 318 - protein, 8 aa, Artificial Sequence
  Description of Artificial Sequence Synthetic peptide
GELYFRAC

SEQ ID NO: 319 - protein, 8 aa, Artificial Sequence
  Description of Artificial Sequence Synthetic peptide
GEIRFALC

SEQ ID NO: 320 - protein, 8 aa, Artificial Sequence
  Description of Artificial Sequence Synthetic peptide
GMIVFPHC

SEQ ID NO: 321 - protein, 8 aa, Artificial Sequence
  Description of Artificial Sequence Synthetic peptide
GEIWFEGC

SEQ ID NO: 322 - protein, 8 aa, Artificial Sequence
  Description of Artificial Sequence Synthetic peptide
GDLKFPLC

SEQ ID NO: 323 - protein, 8 aa, Artificial Sequence
  Description of Artificial Sequence Synthetic peptide
GQILFPVC

SEQ ID NO: 324 - protein, 8 aa, Artificial Sequence
  Description of Artificial Sequence Synthetic peptide
GELFFPKC

SEQ ID NO: 325 - protein, 8 aa, Artificial Sequence
  Description of Artificial Sequence Synthetic peptide
GQIMFPRC

SEQ ID NO: 326 - protein, 8 aa, Artificial Sequence
  Description of Artificial Sequence Synthetic peptide
GSLFFWPC

SEQ ID NO: 327 - protein, 8 aa, Artificial Sequence
  Description of Artificial Sequence Synthetic peptide
GEILFGMC

SEQ ID NO: 328 - protein, 8 aa, Artificial Sequence
  Description of Artificial Sequence Synthetic peptide
GQLKFPFC

SEQ ID NO: 329 - protein, 8 aa, Artificial Sequence
  Description of Artificial Sequence Synthetic peptide
GTIFFRDC

SEQ ID NO: 330 - protein, 8 aa, Artificial Sequence
  Description of Artificial Sequence Synthetic peptide
GQIKFAQC

SEQ ID NO: 331 - protein, 8 aa, Artificial Sequence
  Description of Artificial Sequence Synthetic peptide
GTLIFHHC

SEQ ID NO: 332 - protein, 8 aa, Artificial Sequence
  Description of Artificial Sequence Synthetic peptide
GEIRFGSC

SEQ ID NO: 333 - protein, 8 aa, Artificial Sequence
  Description of Artificial Sequence Synthetic peptide
GQIQFPLC

SEQ ID NO: 334 - protein, 8 aa, Artificial Sequence
  Description of Artificial Sequence Synthetic peptide
GEIKFDHC

SEQ ID NO: 335 - protein, 8 aa, Artificial Sequence
  Description of Artificial Sequence Synthetic peptide
GEIQFGAC

SEQ ID NO: 336 - protein, 8 aa, Artificial Sequence
  Description of Artificial Sequence Synthetic peptide
GELFFEKC

SEQ ID NO: 337 - protein, 8 aa, Artificial Sequence
  Description of Artificial Sequence Synthetic peptide
GEIRFELC

SEQ ID NO: 338 - protein, 8 aa, Artificial Sequence
  Description of Artificial Sequence Synthetic peptide
GEIYFERC

SEQ ID NO: 339 - protein, 9 aa, Artificial Sequence
  Description of Artificial Sequence Synthetic peptide
SGEIYFERC

SEQ ID NO: 340 - protein, 9 aa, Artificial Sequence
  Description of Artificial Sequence Synthetic peptide
AGEIYFERC

SEQ ID NO: 341 - protein, 8 aa, Artificial Sequence
  Description of Artificial Sequence Synthetic peptide
CGEIYFER

SEQ ID NO: 342 - protein, 9 aa, Artificial Sequence
  Description of Artificial Sequence Synthetic peptide
XHXXXXFXC

SEQ ID NO: 343 - protein, 8 aa, Artificial Sequence
  Description of Artificial Sequence Synthetic peptide
SHTRLYFC

SEQ ID NO: 344 - protein, 8 aa, Artificial Sequence
  Description of Artificial Sequence Synthetic peptide
HMRLFFNC

SEQ ID NO: 345 - protein, 8 aa, Artificial Sequence
  Description of Artificial Sequence Synthetic peptide
SHQRLWFC

SEQ ID NO: 346 - protein, 8 aa, Artificial Sequence
  Description of Artificial Sequence Synthetic peptide
HQKMIFAC

SEQ ID NO: 347 - protein, 8 aa, Artificial Sequence
  Description of Artificial Sequence Synthetic peptide
HMRMYFEC

SEQ ID NO: 348 - protein, 8 aa, Artificial Sequence
  Description of Artificial Sequence Synthetic peptide
THQRLWFC

SEQ ID NO: 349 - protein, 7 aa, Artificial Sequence
  Description of Artificial Sequence Synthetic peptide
HQKMIFC

SEQ ID NO: 350 - protein, 8 aa, Artificial Sequence
  Description of Artificial Sequence Synthetic peptide
AIPLFVMC

SEQ ID NO: 351 - protein, 8 aa, Artificial Sequence
  Description of Artificial Sequence Synthetic peptide
KLPLFVMC

SEQ ID NO: 352 - protein, 8 aa, Artificial Sequence
  Description of Artificial Sequence Synthetic peptide
QLPLFVLC

SEQ ID NO: 353 - protein, 8 aa, Artificial Sequence
  Description of Artificial Sequence Synthetic peptide
NDAKIVFC

SEQ ID NO: 354 - protein, 6 aa, Artificial Sequence
  Description of Artificial Sequence Synthetic 6xHis tag
HHHHHH

SEQ ID NO: 355 - protein, 6 aa, Artificial Sequence
  Description of Artificial Sequence Synthetic peptide
DISFRG

SEQ ID NO: 356 - protein, 6 aa, Artificial Sequence
  Description of Artificial Sequence Synthetic peptide
DIGWRG

SEQ ID NO: 357 - protein, 6 aa, Artificial Sequence
  Description of Artificial Sequence Synthetic peptide
SWFRNP

SEQ ID NO: 358 - protein, 5 aa, Artificial Sequence
  Description of Artificial Sequence Synthetic peptide
DKEGR

SEQ ID NO: 359 - protein, 6 aa, Artificial Sequence
  Description of Artificial Sequence Synthetic peptide
EFRHDS

SEQ ID NO: 360 - protein, 6 aa, Artificial Sequence
  Description of Artificial Sequence Synthetic peptide
EFRHDS

SEQ ID NO: 361 - protein, 6 aa, Artificial Sequence
  Description of Artificial Sequence Synthetic peptide
EVHHQK

SEQ ID NO: 362 - protein, 13 aa, Artificial Sequence
  Description of Artificial Sequence Synthetic peptide
EVHHQKLVFFAED

SEQ ID NO: 363 - protein, 7 aa, Artificial Sequence
  Description of Artificial Sequence Synthetic peptide
HQKLVFC

SEQ ID NO: 364 - protein, 6 aa, Artificial Sequence
  Description of Artificial Sequence Synthetic peptide
DREFRH

SEQ ID NO: 365 - protein, 6 aa, Artificial Sequence
  Description of Artificial Sequence Synthetic peptide
DNEFRH

SEQ ID NO: 366 - protein, 6 aa, Artificial Sequence
  Description of Artificial Sequence Synthetic peptide
DDEFRH

SEQ ID NO: 367 - protein, 6 aa, Artificial Sequence
  Description of Artificial Sequence Synthetic peptide
DQEFRH

SEQ ID NO: 368 - protein, 6 aa, Artificial Sequence
  Description of Artificial Sequence Synthetic peptide
DEEFRH

SEQ ID NO: 369 - protein, 6 aa, Artificial Sequence
  Description of Artificial Sequence Synthetic peptide
DGEFRH

SEQ ID NO: 370 - protein, 6 aa, Artificial Sequence
  Description of Artificial Sequence Synthetic peptide
DHEFRH

SEQ ID NO: 371 - protein, 6 aa, Artificial Sequence
  Description of Artificial Sequence Synthetic peptide
DIEFRH

SEQ ID NO: 372 - protein, 6 aa, Artificial Sequence
  Description of Artificial Sequence Synthetic peptide
DLEFRH

SEQ ID NO: 373 - protein, 6 aa, Artificial Sequence
  Description of Artificial Sequence Synthetic peptide
DMEFRH

SEQ ID NO: 374 - protein, 6 aa, Artificial Sequence
  Description of Artificial Sequence Synthetic peptide
DFEFRH

SEQ ID NO: 375 - protein, 6 aa, Artificial Sequence
  Description of Artificial Sequence Synthetic peptide
DPEFRH

SEQ ID NO: 376 - protein, 6 aa, Artificial Sequence
  Description of Artificial Sequence Synthetic peptide
DSEFRH

SEQ ID NO: 377 - protein, 6 aa, Artificial Sequence
  Description of Artificial Sequence Synthetic peptide
DTEFRH

SEQ ID NO: 378 - protein, 6 aa, Artificial Sequence
  Description of Artificial Sequence Synthetic peptide
DWEFRH

SEQ ID NO: 379 - protein, 6 aa, Artificial Sequence
  Description of Artificial Sequence Synthetic peptide
DYEFRH

SEQ ID NO: 380 - protein, 6 aa, Artificial Sequence
  Description of Artificial Sequence Synthetic peptide
DVEFRH

SEQ ID NO: 381 - protein, 6 aa, Artificial Sequence
  Description of Artificial Sequence Synthetic peptide
DAAFRH

SEQ ID NO: 382 - protein, 6 aa, Artificial Sequence
  Description of Artificial Sequence Synthetic peptide
DARFRH

SEQ ID NO: 383 - protein, 6 aa, Artificial Sequence
  Description of Artificial Sequence Synthetic peptide
DANFRH

SEQ ID NO: 384 - protein, 6 aa, Artificial Sequence
  Description of Artificial Sequence Synthetic peptide
DADFRH

SEQ ID NO: 385 - protein, 6 aa, Artificial Sequence
  Description of Artificial Sequence Synthetic peptide
DAQFRH

SEQ ID NO: 386 - protein, 6 aa, Artificial Sequence
  Description of Artificial Sequence Synthetic peptide
DAGFRH

SEQ ID NO: 387 - protein, 6 aa, Artificial Sequence
  Description of Artificial Sequence Synthetic peptide
DAHFRH

SEQ ID NO: 388 - protein, 6 aa, Artificial Sequence
  Description of Artificial Sequence Synthetic peptide
DAIFRH

SEQ ID NO: 389 - protein, 6 aa, Artificial Sequence
  Description of Artificial Sequence Synthetic peptide
DALFRH

SEQ ID NO: 390 - protein, 6 aa, Artificial Sequence
  Description of Artificial Sequence Synthetic peptide
DAMFRH

SEQ ID NO: 391 - protein, 6 aa, Artificial Sequence
  Description of Artificial Sequence Synthetic peptide
DAFFRH

SEQ ID NO: 392 - protein, 6 aa, Artificial Sequence
  Description of Artificial Sequence Synthetic peptide
DAPFRH

SEQ ID NO: 393 - protein, 6 aa, Artificial Sequence
  Description of Artificial Sequence Synthetic peptide
DASFRH

SEQ ID NO: 394 - protein, 6 aa, Artificial Sequence
  Description of Artificial Sequence Synthetic peptide
DATFRH

SEQ ID NO: 395 - protein, 6 aa, Artificial Sequence
  Description of Artificial Sequence Synthetic peptide
DAWFRH

SEQ ID NO: 396 - protein, 6 aa, Artificial Sequence
  Description of Artificial Sequence Synthetic peptide
DAYFRH

SEQ ID NO: 397 - protein, 6 aa, Artificial Sequence
  Description of Artificial Sequence Synthetic peptide
DAVFRH

SEQ ID NO: 398 - protein, 6 aa, Artificial Sequence
  Description of Artificial Sequence Synthetic peptide
DAEARH

SEQ ID NO: 399 - protein, 6 aa, Artificial Sequence
  Description of Artificial Sequence Synthetic peptide
DAERRH

SEQ ID NO: 400 - protein, 6 aa, Artificial Sequence
  Description of Artificial Sequence Synthetic peptide
DAENRH

SEQ ID NO: 401 - protein, 6 aa, Artificial Sequence
  Description of Artificial Sequence Synthetic peptide
DAEDRH

SEQ ID NO: 402 - protein, 6 aa, Artificial Sequence
  Description of Artificial Sequence Synthetic peptide
DAEQRH

SEQ ID NO: 403 - protein, 6 aa, Artificial Sequence
  Description of Artificial Sequence Synthetic peptide
DAEERH

SEQ ID NO: 404 - protein, 6 aa, Artificial Sequence
  Description of Artificial Sequence Synthetic peptide
DAEGRH

SEQ ID NO: 405 - protein, 6 aa, Artificial Sequence
  Description of Artificial Sequence Synthetic peptide
DAEHRH

SEQ ID NO: 406 - protein, 6 aa, Artificial Sequence
  Description of Artificial Sequence Synthetic peptide
DAEIRH

SEQ ID NO: 407 - protein, 6 aa, Artificial Sequence
  Description of Artificial Sequence Synthetic peptide
DAELRH

SEQ ID NO: 408 - protein, 6 aa, Artificial Sequence
  Description of Artificial Sequence Synthetic peptide
DAEMRH

SEQ ID NO: 409 - protein, 6 aa, Artificial Sequence
  Description of Artificial Sequence Synthetic peptide
DAEPRH

SEQ ID NO: 410 - protein, 6 aa, Artificial Sequence
  Description of Artificial Sequence Synthetic peptide
DAESRH

SEQ ID NO: 411 - protein, 6 aa, Artificial Sequence
  Description of Artificial Sequence Synthetic peptide
DAETRH

SEQ ID NO: 412 - protein, 6 aa, Artificial Sequence
  Description of Artificial Sequence Synthetic peptide
DAEWRH

SEQ ID NO: 413 - protein, 6 aa, Artificial Sequence
  Description of Artificial Sequence Synthetic peptide
DAEYRH

SEQ ID NO: 414 - protein, 6 aa, Artificial Sequence
  Description of Artificial Sequence Synthetic peptide
DAEVRH

SEQ ID NO: 415 - protein, 6 aa, Artificial Sequence
  Description of Artificial Sequence Synthetic peptide
DAEFAH

SEQ ID NO: 416 - protein, 6 aa, Artificial Sequence
  Description of Artificial Sequence Synthetic peptide
DAEFNH

SEQ ID NO: 417 - protein, 6 aa, Artificial Sequence
  Description of Artificial Sequence Synthetic peptide
DAEFDH

SEQ ID NO: 418 - protein, 6 aa, Artificial Sequence
  Description of Artificial Sequence Synthetic peptide
DAEFQH

SEQ ID NO: 419 - protein, 6 aa, Artificial Sequence
  Description of Artificial Sequence Synthetic peptide
DAEFEH

SEQ ID NO: 420 - protein, 6 aa, Artificial Sequence
  Description of Artificial Sequence Synthetic peptide
DAEFGH

SEQ ID NO: 421 - protein, 6 aa, Artificial Sequence
  Description of Artificial Sequence Synthetic peptide
DAEFHH

SEQ ID NO: 422 - protein, 6 aa, Artificial Sequence
  Description of Artificial Sequence Synthetic peptide
DAEFIH

SEQ ID NO: 423 - protein, 6 aa, Artificial Sequence
  Description of Artificial Sequence Synthetic peptide
DAEFLH

SEQ ID NO: 424 - protein, 6 aa, Artificial Sequence
  Description of Artificial Sequence Synthetic peptide
DAEFMH

SEQ ID NO: 425 - protein, 6 aa, Artificial Sequence
  Description of Artificial Sequence Synthetic peptide
DAEFFH

SEQ ID NO: 426 - protein, 6 aa, Artificial Sequence
  Description of Artificial Sequence Synthetic peptide
DAEFPH

SEQ ID NO: 427 - protein, 6 aa, Artificial Sequence
  Description of Artificial Sequence Synthetic peptide
DAEFSH

SEQ ID NO: 428 - protein, 6 aa, Artificial Sequence
  Description of Artificial Sequence Synthetic peptide
DAEFTH

SEQ ID NO: 429 - protein, 6 aa, Artificial Sequence
  Description of Artificial Sequence Synthetic peptide
DAEFWH

SEQ ID NO: 430 - protein, 6 aa, Artificial Sequence
  Description of Artificial Sequence Synthetic peptide
DAEFYH

SEQ ID NO: 431 - protein, 6 aa, Artificial Sequence
  Description of Artificial Sequence Synthetic peptide
DAEFVH

SEQ ID NO: 432 - protein, 6 aa, Artificial Sequence
  Description of Artificial Sequence Synthetic peptide
DAEFRA

SEQ ID NO: 433 - protein, 6 aa, Artificial Sequence
  Description of Artificial Sequence Synthetic peptide
DAEFRR

SEQ ID NO: 434 - protein, 6 aa, Artificial Sequence
  Description of Artificial Sequence Synthetic peptide
DAEFRN

SEQ ID NO: 435 - protein, 6 aa, Artificial Sequence
  Description of Artificial Sequence Synthetic peptide
DAEFRD

SEQ ID NO: 436 - protein, 6 aa, Artificial Sequence
  Description of Artificial Sequence Synthetic peptide
DAEFRQ

SEQ ID NO: 437 - protein, 6 aa, Artificial Sequence
  Description of Artificial Sequence Synthetic peptide
DAEFRE

SEQ ID NO: 438 - protein, 6 aa, Artificial Sequence
  Description of Artificial Sequence Synthetic peptide
DAEFRG

SEQ ID NO: 439 - protein, 6 aa, Artificial Sequence
  Description of Artificial Sequence Synthetic peptide
DAEFRI

SEQ ID NO: 440 - protein, 6 aa, Artificial Sequence
  Description of Artificial Sequence Synthetic peptide
DAEFRL

SEQ ID NO: 441 - protein, 6 aa, Artificial Sequence
  Description of Artificial Sequence Synthetic peptide
DAEFRM

SEQ ID NO: 442 - protein, 6 aa, Artificial Sequence
  Description of Artificial Sequence Synthetic peptide
DAEFRF

SEQ ID NO: 443 - protein, 6 aa, Artificial Sequence
  Description of Artificial Sequence Synthetic peptide
DAEFRP

SEQ ID NO: 444 - protein, 6 aa, Artificial Sequence
  Description of Artificial Sequence Synthetic peptide
DAEFRS

SEQ ID NO: 445 - protein, 6 aa, Artificial Sequence
  Description of Artificial Sequence Synthetic peptide
DAEFRT

SEQ ID NO: 446 - protein, 6 aa, Artificial Sequence
  Description of Artificial Sequence Synthetic peptide
DAEFRW

SEQ ID NO: 447 - protein, 6 aa, Artificial Sequence
  Description of Artificial Sequence Synthetic peptide
DAEFRY

SEQ ID NO: 448 - protein, 6 aa, Artificial Sequence
  Description of Artificial Sequence Synthetic peptide
DAEFRV

(19) United States
(12) Patent Application Publication
(10) Pub. No.: US 2013/0287807 A1
(43) Pub. Date: Oct. 31, 2013

(54) COMPOUNDS FOR TREATING SYMPTOMS ASSOCIATED WITH PARKINSON'S DISEASE

(71) Applicants: Markus MANDLER, Vienna (AT); Frank Mattner, Vienna (AT); Walter Schmidt, Vienna (AT)

(72) Inventors: Markus MANDLER, Vienna (AT); Frank Mattner, Vienna (AT); Walter Schmidt, Vienna (AT)

(21) Appl. No.: 13/770,594

(22) Filed: Feb. 19, 2013

Related U.S. Application Data
(62) Division of application No. 12/997,702, filed on Dec. 13, 2010, now abandoned.

(30) Foreign Application Priority Data
Jun. 12, 2008 (AT) A 951/2008
Jun. 12, 2008 (AT) A 952/2008

(51) Int. Cl. C07K 7/06; A61K 39/00

(52) U.S. Cl. CPC C07K 7/06; A61K 39/00
USPC 424/186.1

(57) ABSTRACT

The present invention relates to a compound comprising a peptide for treating, preventing and/or ameliorating motor symptoms of Parkinson's disease, said peptide having a binding capacity to an antibody which is specific for an epitope of the amyloid-beta-peptide (Aβ).

### CROSS-REFERENCE TO RELATED APPLICATIONS

The present application is division of U.S. Ser. No. 12/997,702, filed Dec. 13, 2010, which is a National Stage (371) of PCT/AT2009/000237, filed Jun. 12, 2009, and claims priority to Austrian application A 952/2008, filed Jun. 12, 2008 and Austrian application A 951/2008, filed Jun. 12, 2008.

The present invention relates to methods and means for preventing, ameliorating and treat symptoms associated with Parkinson's disease.

Alzheimer's disease (AD) and Parkinson's Disease (PD) are the most common causes of dementia and movement disorders in humans. While AD is characterized by the accumulation of amyloid-beta protein (forming so called Aβ plaques) which is derived from amyloid precursor protein (APP), PD patients are developing pathologic accumulation of alpha-Synuclein (a-Syn, aSyn; forming so called Lewy Bodies). Both of these molecules are considered to be the major disease causing agents for these neurodegenerative disorders. Both diseases, AD and PD, are associated with degeneration of neurons and synaptic connections, deficiency of specific neurotransmitters, and abnormal accumulation of misfolded proteins, whose non pathogenic paternal proteins play important roles in normal central nervous system functions.

Recently, a novel form of dementia associated with movement disorders but clinical symptoms differing from those of AD, vascular dementia or idiopathic parkinsonism has been defined clinically. This novel syndrome has been defined as dementia with Lewy bodies or Parkinson's with dementia (DLB/PDD). DLB/PDD is amounting to up to 25% of all dementia cases and has to be considered as second most prominent form of dementia in the elderly. The disease is characterized by the formation of widespread Lewy body pathology associated with extensive amyloid deposition. This presence of widespread Lewy bodies differentiates the DLB/PDD cases from all other types of dementia as well as from other movement disorders. The neurological assessment of DLB/PDD shows prominent abnormalities in attention, in executive functions, in memory as well as behavioural and motoric alterations.

It is currently believed that aSyn and Aβ have distinct, as well as convergent, pathogenic effects on the nervous system. Synucleins are believed to affect motoric function more severely than cognitive function, whereas amyloid β peptides are described to have opposite effects. In addition, aSYN and Aβ could interact more directly by engaging synergistic neurodegenerative pathways. It has been recently shown that different pathologic molecules including Aβ, Tau as well as aSyn can mutually exacerbate toxic effects in preclinical disease models and indicate an important function of Aβ in different neurodegenerative conditions. In a recent transgenic animal model for DLB/PDD it has been shown that coexpression of both molecules, haSYN and hAPP, in mice leads to the development of cognitive and motor alterations accompanied by loss of cholinergic neurons and reduction in synaptic vesicles, formation of extensive amyloid plaques, and haSYN-immunoreactive intraneuronal fibrillar inclusions. All of these features are also found in the DLB/PDD syndrome.

Current therapies of symptoms of Parkinson's disease involve the administration of dopaminergic agents to patients suffering from said disease. Dopaminergic agents are believed to reduce the symptoms of Parkinson's disease because it is believed that these symptoms are caused by the deprivation of dopamine in the brain. The insufficiency of dopamine in the brain may therefore be compensated by administering to the patient dopaminergic agents, such as dopamine agonists or dopamine precursors, e.g. levodopa. There is no established cure for Parkinson's disease, which means that the symptoms worsen, necessitating an increase in daily dosage of the medicament as the disease progresses. Furthermore, the chronic use of increased dosages of levodopa leads to the development of motor complications, such as wearing off and involuntary movements (dyskinesia).

The symptoms of motor dysfunction can be improved by levodopa treatment especially combined with other compounds that improve its efficacy.

One of the major disadvantages of the administration of dopaminergic agents is that these agents have to be administered at regular intervals. Furthermore these agents lead only to an increase of dopaminergeic agents in the patient without removing the cause of the symptoms of Parkinson's disease, namely a-Syn plaques.

It is an object of the present invention to provide means for treating symptoms of Parkinson's disease sustainably by reducing the amount of a-Syn deposits.

The present invention relates to a compound comprising a peptide for treating and/or ameliorating motor symptoms of Parkinson's disease, said peptide having a binding capacity to an antibody which is specific for an epitope of the amyloid-beta-peptide (Aβ).

It surprisingly turned out that compounds capable to induce antibodies directed to the amyloid-beta-peptide and, hence, employable to treat beta-amyloidoses such as Alzheimer's disease, can be used to treat and ameliorate the symptoms of Parkinson's disease, in particular the motor symptoms of Parkinson's disease. The antibodies formed by the administration of said compounds reduce surprisingly the amount of a-Syn deposits.

“Motor symptoms”, as used herein, refers to those symptoms of the Parkinson's disease which are described in the EMEA Guideline on Clinical Investigation of Medicinal Products in the Treatment of Parkinson's Disease (CPMP/EWP/563/95 Rev.1) that affect the motor behaviour of a patient suffering from said disease and affects autonomic functions of a patient as well. These symptoms include but are not limited to the core symptoms resting tremor, bradykinesia, rigidity, postural instability as well as stooped posture, dystonia, fatigue, impaired fine motor dexterity and motor coordination, impaired gross motor coordination, poverty of movement (decreased arm swing), akathisia, speech problems, such as softness of voice or slurred speech caused by lack of muscle control, loss of facial expression, or “masking”, micrographia, difficulty swallowing, sexual dysfunction, drooling.

As used herein, the term “epitope” refers to an immunogenic region of an antigen which is recognized by a particular anti-body molecule. An antigen may possess one or more epitopes, each capable of binding an antibody that recognizes the particular epitope.

The term “peptide having a binding capacity to an anti-body which is specific for an epitope of the amyloid-beta-peptide” means that said peptide can be bound to an amyloid-beta peptide specific antibody which has been produced by the administration of amyloid-beta peptide or fragments thereof to a mammal. Said peptide having said binding capacity is able to induce the formation of amyloid-beta peptide specific antibodies in a mammal. The latter antibodies bind consequently to the compound of the present invention as well as to the amyloid-beta peptide.

According to a preferred embodiment of the present invention said epitope of the amyloid-beta-peptide is selected from the group consisting of DAEFRH, EFRHDSGY, pEFRHDSGY, EVHHQKL, HQKLVF and HQKLVFFAED.

It is particularly preferred to use compounds of the present invention which are able to bind to antibodies directed to/specific for the aforementioned naturally occurring epitopes of the amyloid-beta-peptide. Consequently the compound according to the present invention may comprise a peptide having one of said amino acid sequences.

In another embodiment of the present invention the compound of the present invention does preferably not comprise a peptide having the amino acid sequence DAEFRH, EFRHDSGY, pEFRHDSGY, EVHHQKL, HQKLVF and HQKLVFFAED, but, however, also binds to amyloid-beta-specific antibodies.

For identifying such antibody-inducing peptides phage libraries and peptide libraries can be used. Of course it is also possible to identify such peptides by using means of combinatorial chemistry. All of these methods involve the step of contacting a peptide of a pool of peptides with an amyloid-beta peptide specific antibody. The peptides of the pool binding to said antibody can be isolated and sequenced, if the amino acid sequence of the respective peptide is unknown.

In the following peptides are listed which are able to induce the formation of amyloid-beta antibodies in a mammal. These peptides can also be used for reducing symptoms of Parkinson's disease.

According to a preferred embodiment of the present invention the peptide comprises the amino acid sequence

| | |
|---|---|
| X₁X₂X₃X₄X₅X₆X₇, | (Formula I) |

wherein X₁ is G or an amino acid with a hydroxy group or a negatively charged amino acid, preferably glycine (G), glutamic acid (E), tyrosine (Y), serine (S) or aspartic acid (D),

X₂ is a hydrophobic amino acid or a positively charged amino acid, preferably asparagine (N), isoleucine (I), leucine (L), valine (V), lysine (K), tryptophane (W), arginine (R), tyrosine (Y), phenylalanine (F) or alanine (A),

X₃ is a negatively charged amino acid, preferably aspartic acid (D) or glutamic acid (E),

X₄ is an aromatic amino acid or a hydrophobic amino acid or leucine (L), preferably tyrosine (Y), phenylalanine (F) or leucine (L),

X₅ is histidine (H), lysine (K), tyrosine (Y), phenylalanine (F) or arginine (R), preferably histidine (H), phenylalanine (F) or arginine (R), and

X₆ is not present or serine (S), threonine (T), asparagine (N), glutamine (Q), aspartic acid (D), glutamic acid (E), arginine (R), isoleucine (I), lysine (K), tyrosine (Y), or glycine (G), preferably threonine (T), asparagine (N), aspartic acid (D), arginine (R), isoleucine (I) or glycine (G),

X₇ is not present or any amino acid, preferably proline (P), tyrosine (Y), threonine (T), glutamine (Q), alanine (A), histidine (H) or serine (S),

preferably EIDYHR, ELDYHR, EVDYHR, DIDYHR, DLDYHR, DVDYHR, DI-DYRR, DLDYRR, DVDYRR, DKELRI, DWELRI, YREFFI, YREFRI, YAEFRG, EAEFRG, DYEFRG, ELEFRG, DRELRI, DKELKI, DRELKI, GREFRN, EYEFRG, DWEFRDA, SWEFRT, DKELR, SFEFRG, DAEFRWP, DNEFRSP, GSEFRDY, GAEFRFT, SAEFRTQ, SAEFRAT, SWEFRNP, SWEFRLY, SWELRQA, SVEFRYH, SYEFRHH, SQEFRTP, SSEFRVS, DWEFRD, DAELRY, DWELRQ, SLEFRF, GPEFRW, GKEFRT, AYEFRH, DKE(Nle)R, DKE(Nva)R or DKE(Cha)R.

According to a further embodiment of the present invention said peptide comprises the amino acid sequence

| | |
|---|---|
| X₁RX₂DX₃(X₄)ₙ(X₅)ₘ(X₆)ₒ, | (Formula II), |

wherein X₁ is isoleucine (I) or valine (V),

X₂ is tryptophan (W) or tyrosine (Y),

X₃ is threonine (T), valine (V), alanine (A), methionine (M), glutamine (Q) or glycine (G),

X₄ is proline (P), alanine (A), tyrosine (Y), serine (S), cysteine (C) or glycine (G),

X₅ is proline (P), leucine (L), glycine (G) or cysteine (C),

X₆ is cysteine (C),

n, m and o are, independently, 0 or 1,

preferably IRWDTP(C), VRWDVYP(C), IRYDAPL(C), IRYDMAG(C), IRWDTSL(C), IRWDQP(C), IRWDG(C) or IRWDGG(C).

The peptide of the compound of the present invention may comprise the amino acid sequence

| | |
|---|---|
| EX₁WHX₂X₃(X₄)ₙ(X₅)ₘ | (Formula III), |

wherein X₁ is valine (V), arginine (R) or leucine (L),

X₂ is arginine (R) or glutamic acid (E),

X₃ is alanine (A), histidine (H), lysine (K), leucine (L), tyrosine (Y) or glycine (G),

X₄ is proline (P), histidine (H), phenylalanine (F) or glutamine (Q) or Cysteine

X₅ is cysteine (C),

n and m are, independently, 0 or 1,

preferably EVWHRHQ(C), ERHEKH(C), EVWHRLQ(C), ELWHRYP(C), ELWHRAF(C), ELWHRA(C), EVWHRG(C), EVWHRH(C) and ERHEK(C), preferably EVWHRHQ(C), ERHEKH(C), EVWHRLQ(C), ELWHRYP(C) or ELWHRAF(C).

According to a particularly preferred embodiment of the present invention the peptide comprises the amino acid sequence QDFRHY(C), SEFKHG(C), TSFRHG(C), TSVFRH(C), TPFRHT(C), SQFRHY(C), LMFRHN(C), SAFRHH(C), LPFRHG(C), SHFRHG(C), ILFRHG(C), QFKHDL(C), NWFPHP(C), EEFKYS(C), NELRHST(C), GEMRHQP(C), DTYFPRS(C), VELRHSR(C), YSMRHDA(C), AANYFPR(C), SPNQFRH(C), SSSFFPR(C), EDWFFWH(C), SAGSFRH(C), QVMRHHA(C), SEFSHSS(C), QPNLFYH(C), ELFKHHL(C), TLHEFRH(C), ATFRHSP(C), APMYFPH(C), TYFSHSL(C), HEPLFSH(C), SLMRHSS(C), EFLRHTL(C), ATPLFRH(C), QELKRYY(C), THTDFRH(C), LHIPFRH(C), NELFKHF(C), SQYFPRP(C), DEHPFRH(C), MLPFRHG(C), SAMRHSL(C), TPLMFWH(C), LQFKHST(C), ATFRHST(C), TGLMFKH(C), AEFSHWH(C), QSEFKHW(C), AEFMHSV(C), ADHDFRH(C), DGLLFKH(C), IGFRHDS(C), SNSEFRR(C), SELRHST(C), THMEFRR(C), EELRHSV(C), QLFKHSP(C), YEFRHAQ(C), SNFRHSV(C), APIQFRH(C), AYFPHTS(C), NSSELRH(C), TEFRHKA(C), TSTEMWH(C), SQSYFKH(C), (C)SEFKH, SEFKH(C), (C)HEFRH or HEFRH(C).

According to another preferred embodiment of the present invention the peptide comprises the amino acid sequence

| | |
|---|---|
| (X₁)ₘGX₂X₃X₄FX₅X₆(X₇)ₙ | (Formula IV), |

wherein X₁ is serine (S), alanine (A) or cysteine (c),

X₂ is serine (S), threonine (T), glutamic acid (E), aspartic acid (D), glutamine (Q) or methionine (M),

X₃ is isoleucine (I), tyrosine (Y), methionine (M) or leucine (L),

X₄ is leucine (L), arginine (R), glutamine (Q), tryptophan (W), valine (V), histidine (H), tyrosine (Y), isoleucine (I), lysine (K) methionine (M) or phenylalanine (F),

X₅ is alanine (A), phenylalanine (F), histidine (H), asparagine (N), arginine (R), glutamic acid (E), isoleucine (I), glutamine (O), aspartic acid (D), proline (P) or tryptophane (W), glycine (G)

X₆ is any amino acid residue,

X₇ is cysteine (C),

m and n are, independently, 0 or 1,

preferably SGEYVFH(C), SGQLKFP(C), SGQIWFR(C), SGEIHFN(C), GQIWFIS(C), GQIIFQS(C), GQIRFDH(C), GEMWFAL(C), GELQFPP(C), GELWFP(C), GEMQFFI(C), GELYFRA(C), GEIRFAL(C), GMIVFPH(C), GEIWFEG(C), GDLKFPL(C), GQILFPV(C), GELFFPK(C), GQIMFPR(C), GSLFFWP(C), GEILFGM(C), GQLKFPF(C), GTIFFRD(C), GQIKFAQ(C), GTLIFHH(C), GEIRFGS(C), GQIQFPL(C), GEIKFDH(C), GEIQFGA(C), GELFFEK(C), GEIRFEL(C), GEIYFER(C), SGEIYFER(C), AGEIYFER(C) or (C)GEIYFER.

According to a further preferred embodiment of the present invention the peptide comprises the amino acid sequence

| | |
|---|---|
| (X₁)ₘHX₂X₃X₄X₅FX₆(X₇)ₙ | (Formula V), |

wherein X₁ is serine (S), threonine (T) or cysteine (C),

X₂ is glutamine (Q), threonine (T) or methionine (M),

X₃ is lysine (K) or arginine (R),

X₄ is leucine (L), methionine (M),

X₅ is tryptophane (W), tyrosine (Y), phenylalanine (F) or isoleucine (I),

X₆ is asparagine (N), glutamic acid (E), alanine (A) or cysteine (C),

X₇ is cysteine (C),

n and m are, independently, 0 or 1,

preferably SHTRLYF(C), HMRLFFN(C), SHQRLWF(C), HQKMIFA(C), HMRMYFE(C), THQRLWF(C) or HQKMIF(C).

According to a preferred embodiment of the present invention the peptide comprises the amino acid sequence AIPLFVM(C), KLPLFVM(C), QLPLFVL(C) or NDAKIVF(C).

The compound according to the present invention is preferably a polypeptide/peptide and comprises 4 to 30 amino acid residues, preferably 5 to 25 amino acid residues, more preferably 5 to 20 amino acid residues.

The compound of the present invention may also be part of a polypeptide comprising 4 to 30 amino acid residues.

The peptides exhibiting an affinity to amyloid-beta antibodies may be considered as mimotopes. According to the present invention the term “mimotope” refers to a molecule which has a conformation that has a topology equivalent to the epitope of which it is a mimic. The mimotope binds to the same antigen-binding region of an antibody which binds immunospecifically to a desired antigen. The mimotope will elicit an immunological response in a host that is reactive to the antigen to which it is a mimic. The mimotope may also act as a competitor for the epitope of which it is a mimic in in vitro inhibition assays (e.g. ELISA inhibition assays) which involve the epitope and an antibody binding to said epitope. However, a mimotope of the present invention may not necessarily prevent or compete with the binding of the epitope of which it is a mimic in an in vitro inhibition assay although it is capable to induce a specific immune response when administered to a mammal. The compounds of the present invention comprising such mimotopes (also those listed above) have the advantage to avoid the formation of autoreactive T-cells, since the peptides of the compounds have an amino acid sequence which varies from those of naturally occurring amyloid-beta peptide.

The mimotopes/peptides of the present invention can be synthetically produced by chemical synthesis methods which are well known in the art, either as an isolated peptide or as a part of another peptide or polypeptide. Alternatively, the peptide mimotope can be produced in a microorganism which produces the peptide mimotope which is then isolated and if desired, further purified. The peptide mimotope can be produced in microorganisms such as bacteria, yeast or fungi, in eukaryote cells such as a mammalian or an insect cell, or in a recombinant virus vector such as adenovirus, poxvirus, herpesvirus, Simliki forest virus, baculovirus, bacteriophage, sindbis virus or sendai virus. Suitable bacteria for producing the peptide mimotope include E. coli, B. subtilis or any other bacterium that is capable of expressing peptides such as the peptide mimotope. Suitable yeast types for expressing the peptide mimotope include Saccharomyces cerevisiae, Schizosaccharomyces pombe, Candida, Pichia pastoris or any other yeast capable of expressing peptides. Corresponding methods are well known in the art. Also methods for isolating and purifying recombinantly produced peptides are well known in the art and include e.g. as gel filtration, affinity chromatography, ion exchange chromatography etc.

To facilitate isolation of the peptide mimotope, a fusion polypeptide may be made wherein the peptide mimotope is translationally fused (covalently linked) to a heterologous polypeptide which enables isolation by affinity chromatography. Typical heterologous polypeptides are His-Tag (e.g. His₆; 6 histidine residues), GST-Tag (Glutathione-S-transferase) etc. The fusion polypeptide facilitates not only the purification of the mimotopes but can also prevent the mimotope polypeptide from being degraded during purification. If it is desired to remove the heterologous polypeptide after purification the fusion polypeptide may comprise a cleavage site at the junction between the peptide mimotope and the heterologous polypeptide. The cleavage site consists of an amino acid sequence that is cleaved with an enzyme specific for the amino acid sequence at the site (e.g. proteases).

The mimotopes of the present invention may also be modified at or nearby their N- and/or C-termini so that at said positions a cysteine residue is bound thereto. In a preferred embodiment terminally positioned (located at the N- and C-termini of the peptide) cysteine residues are used to cyclize the peptides through a disulfide bond.

The mimotopes of the present invention may also be used in various assays and kits, in particular in immunological assays and kits. Therefore, it is particularly preferred that the mimotope may be part of another peptide or polypeptide, particularly an enzyme which is used as a reporter in immunological assays. Such reporter enzymes include e.g. alkaline phosphatase or horseradish peroxidase.

The mimotopes according to the present invention preferably are antigenic polypeptides which in their amino acid sequence vary from the amino acid sequence of Aβ or of fragments of Aβ. In this respect, the inventive mimotopes may not only comprise amino acid substitutions of one or more naturally occurring amino acid residues but also of one or more non-natural amino acids (i.e. not from the 20 “classical” amino acids) or they may be completely assembled of such non-natural amino acids. Moreover, the inventive antigens which induce antibodies directed and binding to Aβ1-40/42, AβpE3-40/42, Aβ3-40/42, Aβ11-40/42, AβpE11-40/42 and Aβ14-40/42 (and other N-terminally truncated forms of Aβ starting from amino acid positions 2, 3, 4, 5, 6, 7, 8, 9, 10, 11, 12 and 13) may be assembled of D- or L-amino acids or of combinations of DL-amino acids and, optionally, they may have been changed by further modifications, ring closures or derivatizations. Suitable antibody-inducing antigens may be provided from commercially available peptide libraries. Preferably, these peptides are at least 7 amino acids, and preferred lengths may be up to 16, preferably up to 14 or 20 amino acids (e.g. 5 to 16 amino acid residues). According to the invention, however, also longer peptides may very well be employed as antibody-inducing antigens. Furthermore the mimotopes of the present invention may also be part of a polypeptide and consequently comprising at their N- and/or C-terminus at least one further amino acid residue.

For preparing the mimotopes of the present invention (i.e. the antibody-inducing antigens disclosed herein), of course also phage libraries, peptide libraries are suitable, for instance produced by means of combinatorial chemistry or obtained by means of high throughput screening techniques for the most varying structures (Display: A Laboratory Manual by Carlos F. Barbas (Editor), et al.; Willats W G Phage display: practicalities and prospects. Plant Mol. Biol. 2002 December; 50(6):837-54).

Furthermore, according to the invention also anti-Aβ1-40/42-, -AβpE3-40/42-, -Aβ3-40/42-, -Aβ11-40/42-AβpE11-40/42- and Aβ14-40/42-antibody-inducing antigens based on nucleic acids (“aptamers”) may be employed, and these, too, may be found with the most varying (oligonucleotide) libraries (e.g. with 2-180 nucleic acid residues) (e.g. Burgstaller et al., Curr. Opin. Drug Discov. Dev. 5(5) (2002), 690-700; Famulok et al., Acc. Chem. Res. 33 (2000), 591-599; Mayer et al., PNAS 98 (2001), 4961-4965, etc.). In antibody-inducing antigens based on nucleic acids, the nucleic acid backbone can be provided e.g. by the natural phosphor-diester compounds, or also by phosphorotioates or combinations or chemical variations (e.g. as PNA), wherein as bases, according to the invention primarily U, T, A, C, G, H and mC can be employed. The 2′-residues of the nucleotides which can be used according to the present invention preferably are H, OH, F, Cl, NH₂, O-methyl, O-ethyl, O-propyl or O-butyl, wherein the nucleic acids may also be differently modified, i.e. for instance with protective groups, as they are commonly employed in oligonucleotide synthesis. Thus, aptamer-based antibody-inducing antigens are also preferred antibody-inducing antigens within the scope of the present invention.

According to a preferred embodiment of the present invention the compound is coupled to a pharmaceutically acceptable carrier, preferably KLH (Keyhole Limpet Hemocyanin), tetanus toxoid, albumin-binding protein, bovine serum albumin, a dendrimer (MAP; Biol. Chem. 358: 581), peptide linkers (or flanking regions) as well as the adjuvant substances described in Singh et al., Nat. Biotech. 17 (1999), 1075-1081 (in particular those in Table 1 of that document), and O'Hagan et al., Nature Reviews, Drug Discovery 2 (9) (2003), 727-735 (in particular the endogenous immunopotentiating compounds and delivery systems described therein), or mixtures thereof. The conjugation chemistry (e.g. via heterobifunctional compounds such as GMBS and of course also others as described in “Bioconjugate Techniques”, Greg T. Hermanson) in this context can be selected from reactions known to the skilled man in the art. Moreover, the vaccine composition may be formulated with an adjuvant, preferably a low soluble aluminium composition, in particular aluminium hydroxide. Of course, also adjuvants like MF59 aluminium phosphate, calcium phosphate, cytokines (e.g., IL-2, IL-12, GM-CSF), saponins (e.g., QS21), MDP derivatives, CpG oligos, LPS, MPL, polyphosphazenes, emulsions (e.g., Freund's, SAF), liposomes, virosomes, iscoms, cochleates, PLG microparticles, poloxamer particles, virus-like particles, heat-labile enterotoxin (LT), cholera toxin (CT), mutant toxins (e.g., LTK63 and LTR72), microparticles and/or polymerized liposomes may be used.

The compound of the present invention is preferably bound to the carrier or adjuvant via a linker, which is selected from the group consisting of NHS-poly (ethylene oxide) (PEO) (e.g. NHS-PEO₄-maleimide).

A vaccine which comprises the present compound (mimotope, peptide) and the pharmaceutically acceptable carrier may be administered by any suitable mode of application, e.g. i.d., i.v., i.p., i.m., intranasally, orally, subcutaneously, etc. and in any suitable delivery device (O'Hagan et al., Nature Reviews, Drug Discovery 2 (9), (2003), 727-735). The compound of the present invention is preferably formulated for intravenous, subcutaneous, intradermal or intramuscular administration (see e.g. “Handbook of Pharmaceutical Manufacturing Formulations”, Sarfaraz Niazi, CRC Press Inc, 2004).

The medicament (vaccine) according to the present invention contains the compound according to the invention in an amount of from 0.1 ng to 10 mg, preferably 10 ng to 1 mg, in particular 100 ng to 100 μg, or, alternatively, e.g. 100 fmol to 10 μmol, preferably 10 μmol to 1 μmol, in particular 100 μmol to 100 nmol. Typically, the vaccine may also contain auxiliary substances, e.g. buffers, stabilizers etc.

According to a preferred embodiment of the present invention the motor symptoms of Parkinson's disease are selected from the group consisting of resting tremor, Bradykinesia, rigidity, postural instability, stooped posture, dystonia, fatigue, impaired fine motor dexterity and motor coordination, impaired gross motor coordination, poverty of movement (decreased arm swing), akathisia, speech problems, loss of facial expression, micrographia, difficulty swallowing, sexual dysfunction and drooling.

Another aspect of the present invention relates to the use of a compound according to the present invention for the manufacture of a medicament for treating, preventing and/or ameliorating motor symptoms of Parkinson's disease.

Yet another aspect of the present invention relates to a method for treating and/or ameliorating symptoms, in particular motor symptoms, of Parkinson's disease.

### BRIEF DESCRIPTION OF THE DRAWINGS

The present invention is further illustrated in the following figures and examples, however, without being restricted thereto.

FIG. 1A to FIG. 1C shows the individualised peptide members of library 4 used for the present screening process.

FIG. 2 shows an inhibition assay with mimotopes for DAEFRH.

FIG. 3 shows another inhibition assay with other mimotopes for DAEFRH.

FIGS. 4 and 5 describe the results of inhibition assays performed with mimotope peptides according to the present invention.

FIGS. 6 to 9 show the results of inhibition assays performed with mimotope peptides 4011-4018, 4019-4025, 4031-4038 and 4061-4064, respectively.

FIG. 10 shows binding of monoclonal antibody MV-001 to specific peptides and recombinant proteins;

FIG. 11 shows binding of monoclonal antibody MV-003 to specific peptides and recombinant proteins;

FIG. 12 shows binding of monoclonal antibody MV-004 to specific peptides and recombinant proteins;

FIG. 13A to FIG. 13C shows typical binding assays with mimotopes for β-amyloid and N-terminally truncated and/or posttrans-lationally modified β-amyloid fragments;

FIG. 14A to FIG. 14C shows typical inhibition assays with mimotopes for β-amyloid and N-terminally truncated and/or posttrans-lationally modified β-amyloid fragments;

FIG. 15A to FIG. 15C shows examples for in vivo characterisations of the immune response elicited by mimotope vaccination (injected peptide/irrelevant peptide);

FIG. 16A to FIG. 16C shows examples for in vivo characterisation of the immune response elicited by mimotope vaccination against Amyloid Beta fragments;

FIG. 17A to FIG. 17B shows examples for in vivo characterisation of the immune response elicited by mimotope vaccination against full length

FIG. 18 shows areas occupied by amyloid plaques. Tg2576 were injected 6 times with mimotope vaccines adjuvanted with aluminium hydroxide (ALUM) by s.c. inoculation at monthly intervals. Control mice received PBS-ALUM only. Area occupied by amyloid plaques shown as percent of the control group. Gr1 . . . control group; Gr2 . . . received p4381; Gr3 . . . received p4390; Gr4 . . . received p4715

FIG. 19 shows areas occupied by amyloid plaques. Tg2576 were injected 6 times with AFFITOPE vaccines adjuvanted with aluminium hydroxide (ALUM) by s.c. inoculation at monthly intervals. Control mice received PBS-ALUM only. Area occupied by amyloid plaques shown as percent of the control group. Gr1 . . . control group; Gr2 . . . received p4395.

FIG. 20 shows binding of monoclonal antibody MV-002 to specific peptides and recombinant proteins.

FIG. 21 shows typical binding assays with mimotopes for β-amyloid and N-terminally truncated and/or posttrans-lationally modified β-amyloid fragments.

FIG. 22 shows typical inhibition assays with mimotopes for β-amyloid and N-terminally truncated and/or posttrans-lationally modified β-amyloid fragments.

FIG. 23 shows examples for in vivo characterisations of the immune response elicited by mimotope vaccination (injected peptide/irrelevant peptide).

FIG. 24 shows examples for in vivo characterisation of the immune response elicited by mimotope vaccination against Amyloid Beta fragments and sAPP-alpha.

FIG. 25 shows examples for in vivo characterisation of the immune response elicited by mimotope vaccination against full length Aβ40/42.

FIG. 26 shows areas occupied by amyloid plaques. Tg2576 were injected 6 times with mimotope vaccines adjuvanted with aluminium hydroxide (ALUM) by s.c. inoculation at monthly intervals. Control mice received PBS-ALUM only. Area occupied by amyloid plaques shown as percent of the control group. Gr1 . . . control group; Gr2 . . . received p4675.

FIG. 27 shows a-synuclein positive inclusions. A . . . Control treated animal; B . . . AD mimotope treated animal; A and B display cortical sections stained for a-synuclein. Positive staining shows neuronal cells including pyramidal and non-pyramidal neurons. Arrows indicate two typical examples for inclusions in A and B. C . . . Number of inclusions in cortex and hippocampus (indicated as cortex).

FIG. 28 shows neuronal density. Pictures display cortical sections stained for NeuN. positive staining shows neuronal cells including pyramidal and non-pyramidal neurons. A . . . indicates a control treated animal; B . . . Shows an AD mimotope treated animal respectively. C and D . . . shows the number of NeuN positive neurons in the cortex and hippocampus.

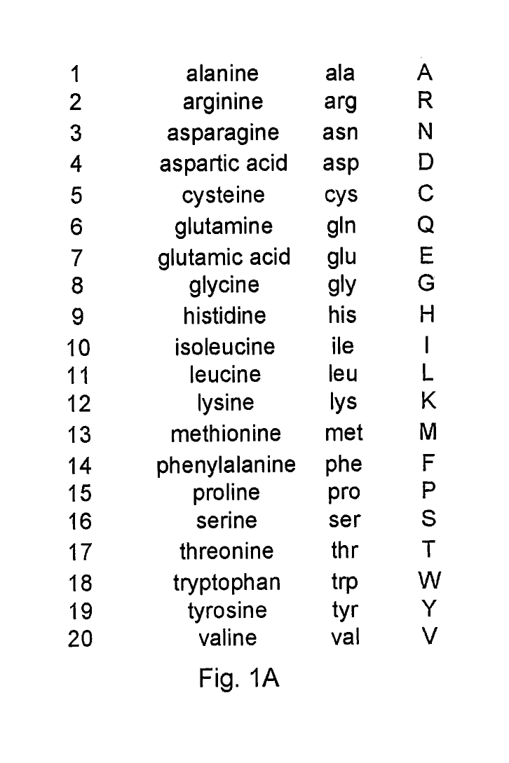

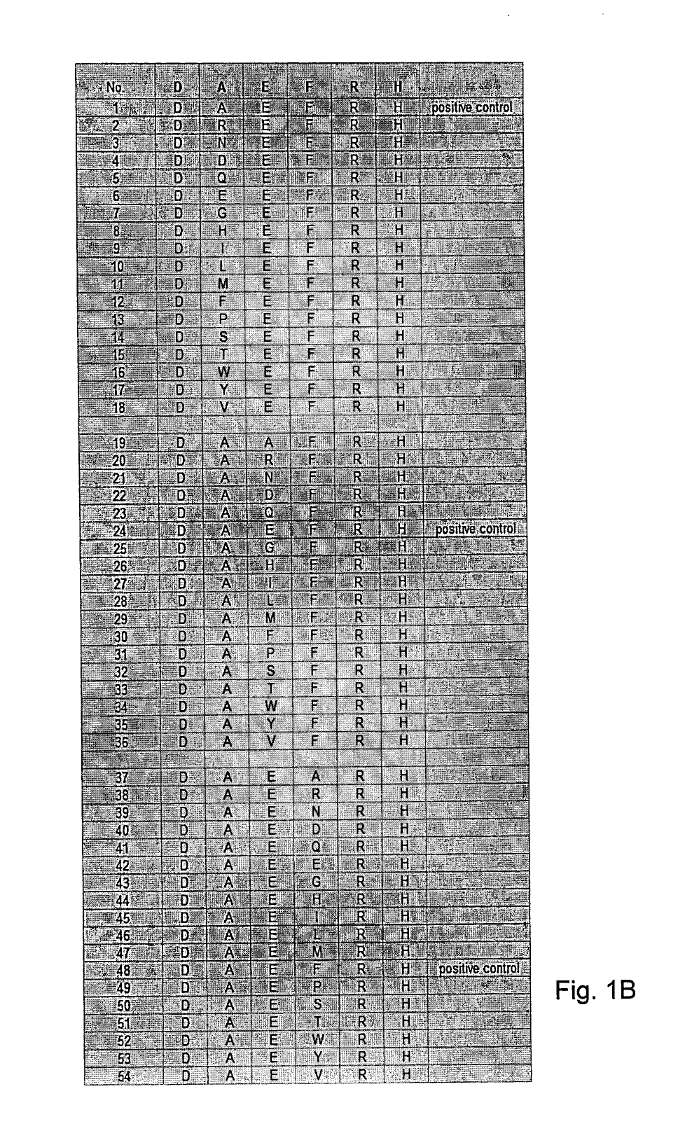

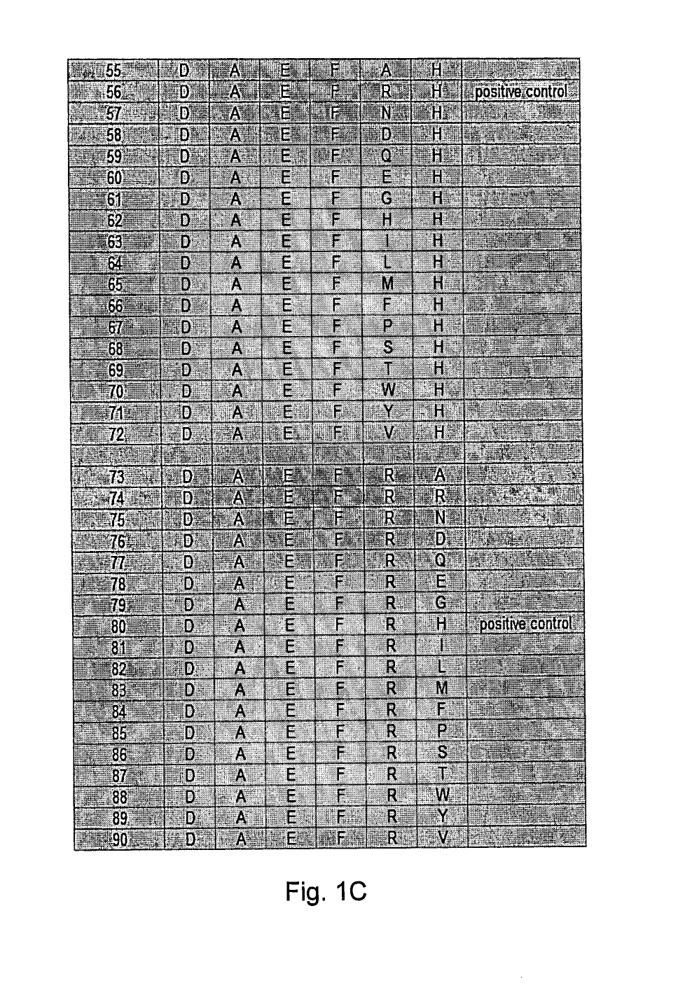

### EXAMPLES

### Example 1

### Generation of Monoclonal Antibodies (mAb) to Detect Aβ42-Derived Peptide Species with Free N-Terminus (Free Aspartic Acid at the N-Terminus)

Mice are vaccinated with the 6mer peptide DAEFRH (natural N-terminal Aβ42 sequence) linked to the protein bovine serum albumin BSA (to make use of the hapten-carrier-effect), emulsified in CFA (first injection) and IFA (booster injections). DAEFRH-peptide-specific, antibody-producing hybridomas are detected by ELISA (DAEFRH-peptide-coated ELISA plates). Peptide SEVKMDAEFRH (natural N-terminally prolonged sequence, APP-derived, containing the Aβ42-derived sequence DAEFRH) is used as negative control peptide: hybridomas recognizing the prolonged peptide are excluded because they do not distinguish between Aβ42-derived peptides with free aspartic acid at the N-terminus and APP-derived peptide DAEFRH without free aspartic acid.

### Example 2

### Identifying Mimotopes by Inhibition Assay

3.1. Libraries

The peptide libraries employed in inhibition assays (see below) are disclosed in WO 2004/062556.

3.2. Inhibition Assay

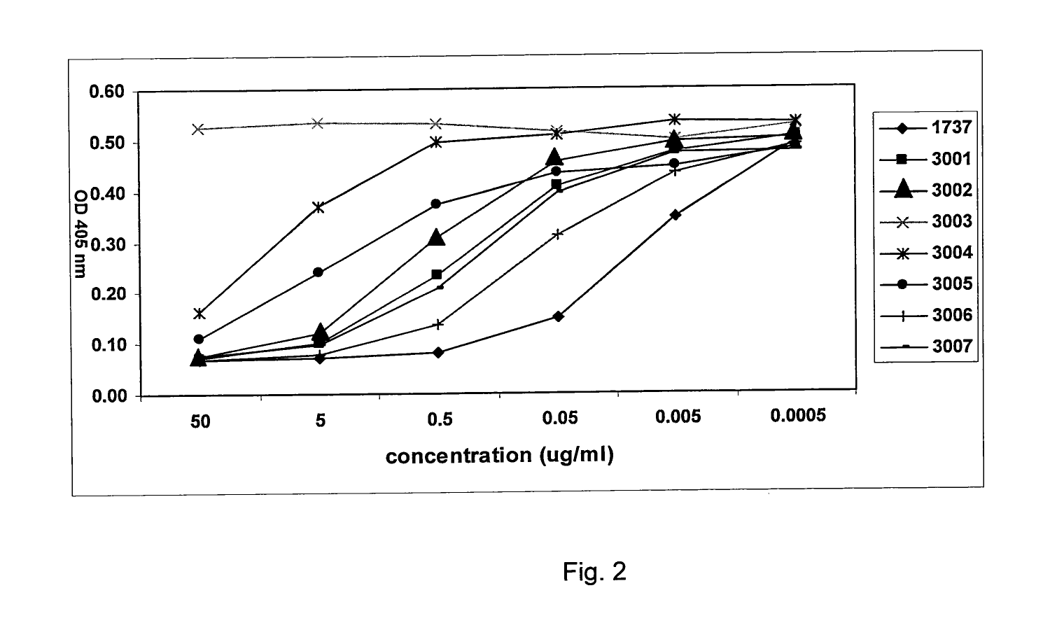

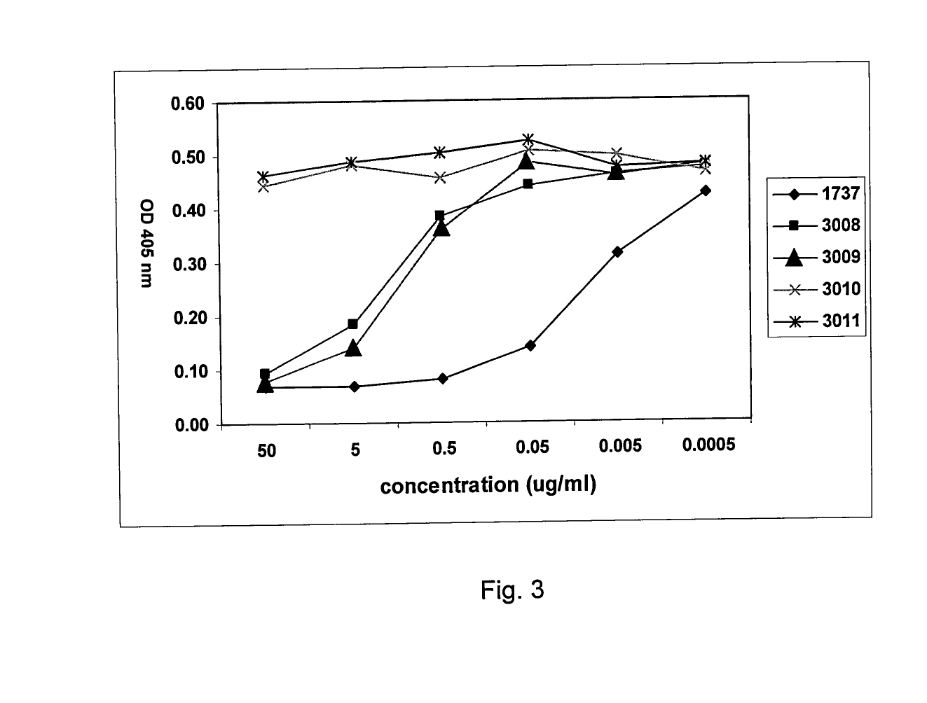

FIGS. 2 and 3 describe the results of inhibition assays performed with mimotope peptides included in and obtained from the 5 libraries (as described in WO 2004/062556). The mimotope peptides compete with the original epitope for recognition by the monoclonal antibody. Original epitope and mimotope peptides contain an additional C at the C-terminus for coupling to a protein carrier (if desired).

The following peptides are used:

| | | |
|---|---|---|
| Peptide | 1737 | DAEFRH |
| Peptide | 3001 | DKELRI |
| Peptide | 3002 | DWELRI |
| Peptide | 3003 | YREFFI |
| Peptide | 3004 | YREFRI |
| Peptide | 3005 | YAEFRG |
| Peptide | 3006 | EAEFRG |
| Peptide | 3007 | DYEFRG |
| Peptide | 3008 | ELEFRG |
| Peptide | 3009 | SFEFRG |
| Peptide | 3010 | DISFRG |
| Peptide | 3011 | DIGWRG |

Procedure:

ELISA plates (Nunc Maxisorp) are coated with the original peptide epitope DAEFRH(C-terminally prolonged with C and coupled to bovine serum albumin BSA) at a concentration of 0.1 μg/ml peptide-BSA (100 μl/well, 12 h, 4° C.). After blocking with PBS/BSA 1% (200 μl/well, 12 h, 4° C.), the plates are washed 3× times with PBS/Tween. Then, biotinylated monoclonal antibody (1:2000, 50 μl/well) and peptides (50 μl/well) at 50, 5, 0.5, 0.05, 0.005, and 0.0005 μg/ml are added for 20 min. at 37° C. The plates are washed 3× times with PBS/Tween and are incubated with horseradish peroxidase (HRP)-labeled streptavidin (100 μl/well, 30 min, RT). The plates are washed 5× times with PBS/Tween and are incubated with ABTS+H₂O₂(0.1% w/v, 10 to 45 min) and the reaction is stopped with citric acid followed by photometric evaluation (wavelength 405 nm).

As expected and seen in FIG. 2, peptide 1737 DAEFRH can compete with BSA-coupled, plate-bound peptide DAEFRH and thus inhibits recognition by the monoclonal antibody. Furthermore, it is shown that peptide 3003 is not able to inhibit binding of the monoclonal antibody to the original epitope. In contrast, peptides 3001, 3002, 3004, 3005, 3006, and 3007 (to a different extent) block epitope recognition. Whereas peptide 3004 is only inhibitory at a high concentration (50 μg/ml), peptides 3001, 3006, and 3007 are strongly inhibitory with an IC₅₀ of less than 0.5 μg/ml. Peptides 3002 and 3005 are “intermediate” inhibitors with an IC₅₀ of more than 0.5 μg/ml.

As expected and seen in FIG. 3, peptide 1737 DAEFRH can successfully compete with BSA-coupled, plate-bound peptide DAEFRH for monoclonal antibody recognition in an additionally performed, independent experiment. Furthermore, it is shown that peptides 3010 and 3011 are not inhibitory at the concentrations tested, whereas peptides 3008 and 3009 are (relatively) weak inhibitors with an IC₅₀ of less than 5 μg/ml.

Table 1 briefly summarizes the inhibitory capacity of mimotopes included in and obtained from libraries (as described):

TABLE 1
Inhibitory capacity of mimotopes:

| | | | |
|---|---|---|---|
| Peptide | 3001 | DKELRI | strong |
| Peptide | 3002 | DWELRI | intermediate |
| Peptide | 3003 | YREFFI | none |
| Peptide | 3004 | YREFRI | weak |
| Peptide | 3005 | YAEFRG | intermediate |
| Peptide | 3006 | EAEFRG | strong |
| Peptide | 3007 | DYEFRG | strong |
| Peptide | 3008 | ELEFRG | weak |
| Peptide | 3009 | SFEFRG | weak |
| Peptide | 3010 | DISFRG | none |
| Peptide | 3011 | DIGWRG | none |

### Example 3

### Inhibition Assay for Additional Mimotopes Screenend According to the Present Invention

Inhibition Assay

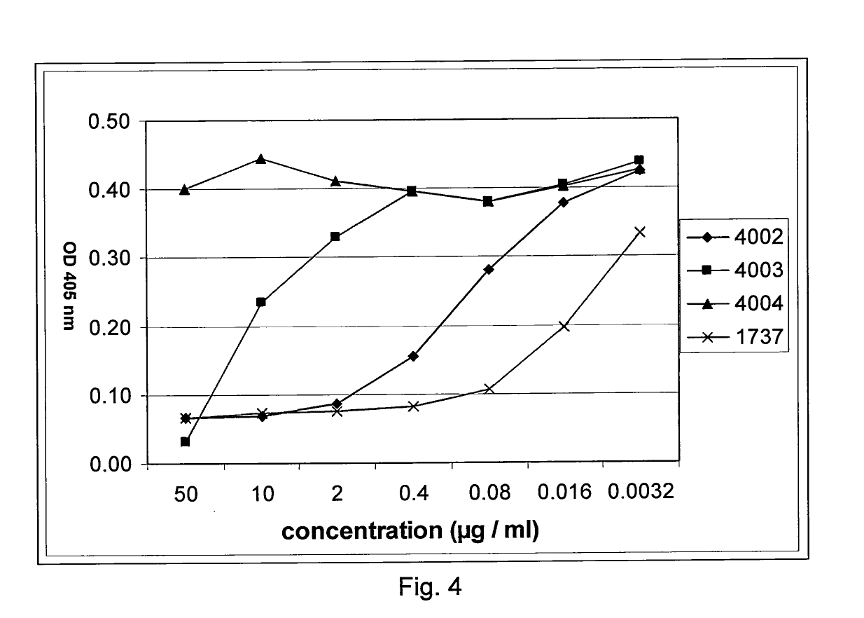

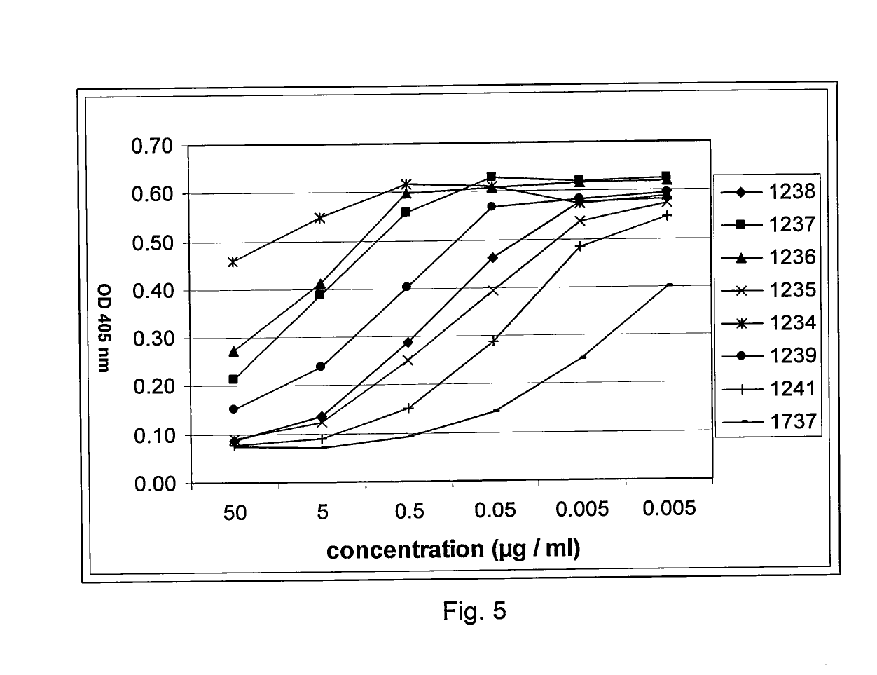

FIGS. 4 and 5 describe the results of inhibition assays performed with mimotope peptides included in and obtained from the 5 libraries as described in WO 2004/062556. The mimotope peptides compete with the original epitope for recognition by the monoclonal antibody. Original epitope and mimotope peptides contain an additional C at the C-terminus (position 7) for coupling to a protein carrier (if desired).

The following peptides are used:

| | | |
|---|---|---|
| Peptide | 1737 | DAEFRH (original epitope + C) |
| Peptide | 1234 | KKELRI |
| Peptide | 1235 | DRELRI |
| Peptide | 1236 | DKELKI |
| Peptide | 1237 | DRELKI |
| Peptide | 1238 | DKELR |
| Peptide | 1239 | EYEFRG |
| Peptide | 1241 | DWEFRDA |
| Peptide | 4002 | SWEFRT |
| Peptide | 4003 | GREFRN |
| Peptide | 4004 | WHWSWR |

Procedure:

ELISA plates (Nunc Maxisorp) are coated with the original peptide epitope DAEFRH(C-terminally prolonged with C and coupled to bovine serum albumin BSA) at a concentration of 0.1 μg/ml peptide-BSA (100 μl/well, 12 h, 4° C.). After blocking with PBS/BSA 1% (200 μl/well, 12 h, 4° C.), the plates are washed 3× times with PBS/Tween. Then, biotinylated monoclonal antibody (1:2000, 50 μl/well) and peptides (50 μl/well) at different concentrations are added for 20 min. at 37° C. The plates are washed 3× times with PBS/Tween and are incubated with horseradish peroxidase (HRP)-labeled streptavidin (100 μl/well, 30 min, RT). The plates are washed 5× times with PBS/Tween and are incubated with ABTS+H₂O₂ (0.1% w/v, 10 to 45 min) and the reaction is stopped with citric acid followed by photometric evaluation (wavelength 405 nm).

As expected and seen in FIG. 4, peptide 1737 DAEFRH can compete with BSA-coupled, plate-bound peptide DAEFRH and thus inhibits recognition by the monoclonal antibody. Furthermore, it is shown that peptide 4004 is not able to inhibit binding of the monoclonal antibody to the original epitope. In contrast, peptides 4002 and 4003 (to a different extent) block epitope recognition. Whereas peptide 4003 is only inhibitory at a relatively high concentration (10 μg/ml), peptide 4002 is strongly inhibitory with an IC₅₀ of less than 0.4 μg/ml.

As expected and seen in FIG. 5, peptide 1737 DAEFRH can successfully compete with BSA-coupled, plate-bound peptide DAEFRH for monoclonal antibody recognition in an additionally performed, independent experiment. Furthermore, it is shown that peptide 1234 is hardly inhibitory at the concentrations tested, whereas peptides 1235, 1236, 1237, 1238, 1239 and 1241 (to a different extent) block epitope recognition. Peptides 1235, 1238 and 1241 are strong inhibitors with an IC₅₀ of less than 0.5 μg/ml, whereas peptides 1236 and 1237 are (relatively) weak inhibitors with an IC₅₀ of more than 5 μg/ml. Peptide 1239 is an intermediate inhibitor with an IC₅₀ of more than 0.5 μg/ml.

Table 2 briefly summarizes the inhibitory capacity of mimotopes included in and obtained from libraries (as described):

TABLE 2
Inhibitory capacity of mimotopes:

| | |
|---|---|
| Peptide 1234 KKELRI | none |
| Peptide 1235 DRELRI | strong |
| Peptide 1236 DKELKI | weak |
| Peptide 1237 DRELKI | weak |
| Peptide 1238 DKELR | strong |
| Peptide 1239 EYEFRG | intermediate |
| Peptide 1241 DWEFRDA | strong |
| Peptide 4002 SWEFRT | strong |
| Peptide 4003 GREFRN | weak |
| Peptide 4004 WHWSWR | none |

The results presented in FIGS. 4 and 5 show that in addition to various 6mer peptides (as shown here and before), 5mer peptides (namely peptide 1238 DKELR) and 7mer peptides (namely peptide 1241 DWEFRDA) may be used as epitopes in a mimotope-based Alzheimer vaccine.

### Example 4

### Inhibition Assay for Mimotopes of the Present Invention and Disclosed in WO 2006/005707

### Libraries:

The mimotopes are obtained as described in WO 2006/005707.

The following peptides are used for the following assays:

| Peptide 1737 DAEFRH | original epitope | |
|---|---|---|
| Peptide 4011 DAEFRWP | 7mer | s |
| Peptide 4012 DNEFRSP | 7mer | s |
| Peptide 4013 GSEFRDY | 7mer | m |
| Peptide 4014 GAEFRFT | 7mer | m |
| Peptide 4015 SAEFRTQ | 7mer | s |
| Peptide 4016 SAEFRAT | 7mer | s |
| Peptide 4017 SWEFRNP | 7mer | s |
| Peptide 4018 SWEFRLY | 7mer | s |
| Peptide 4019 SWFRNP | 6mer | — |
| Peptide 4020 SWELRQA | 7mer | s |
| Peptide 4021 SVEFRYH | 7mer | s |
| Peptide 4022 SYEFRHH | 7mer | s |
| Peptide 4023 SQEFRTP | 7mer | s |
| Peptide 4024 SSEFRVS | 7mer | s |
| Peptide 4025 DWEFRD | 6mer | s |
| Peptide 4031 DAELRY | 6mer | s |
| Peptide 4032 DWELRQ | 6mer | s |
| Peptide 4033 SLEFRF | 6mer | s |
| Peptide 4034 GPEFRW | 6mer | s |
| Peptide 4035 GKEFRT | 6mer | s |
| Peptide 4036 AYEFRH | 6mer | m |
| Peptide 4037 VPTSALA | 7mer | — |
| Peptide 4038 ATYAYWN | 7mer | — |

Furthermore, the following 5mer peptides (with non natural amino acids) are used for inhibition assays:

| | | |
|---|---|---|
| Peptide 4061 DKE(tBuGly)R | 5mer | — |
| Peptide 4062 DKE(Nle)R | 5mer | m |
| Peptide 4063 DKE(Nva)R | 5mer | m |
| Peptide 4064 DKE((Cha)R | 5mer | m |

(s: strong inhibition, m: moderate inhibition; -: no inhibition)

Procedure:

ELISA plates (Nunc Maxisorp) are coated with the original peptide epitope DAEFRH(C-terminally prolonged with C and coupled to bovine serum albumin BSA) at a concentration of 0.1 μg/ml peptide-BSA (100 μl/well, 12 h, 4° C.). After blocking with PBS/BSA 1% (200 μl/well, 12 h, 4° C.), the plates are washed 3× times with PBS/Tween. Then, biotinylated monoclonal antibody (1:2000, 50 μl/well) and peptides (50 μl/well) at different concentrations are added for 20 min. at 37° C. The plates are washed 3× times with PBS/Tween and are incubated with horseradish peroxidase (HRP)-labeled streptavidin (100 μl/well, 30 min, RT). The plates are washed 5× times with PBS/Tween and are incubated with ABTS+H₂O₂(0.1% w/v, 10 to 45 min) and the reaction is stopped with citric acid followed by photometric evaluation (wavelength 405 nm).

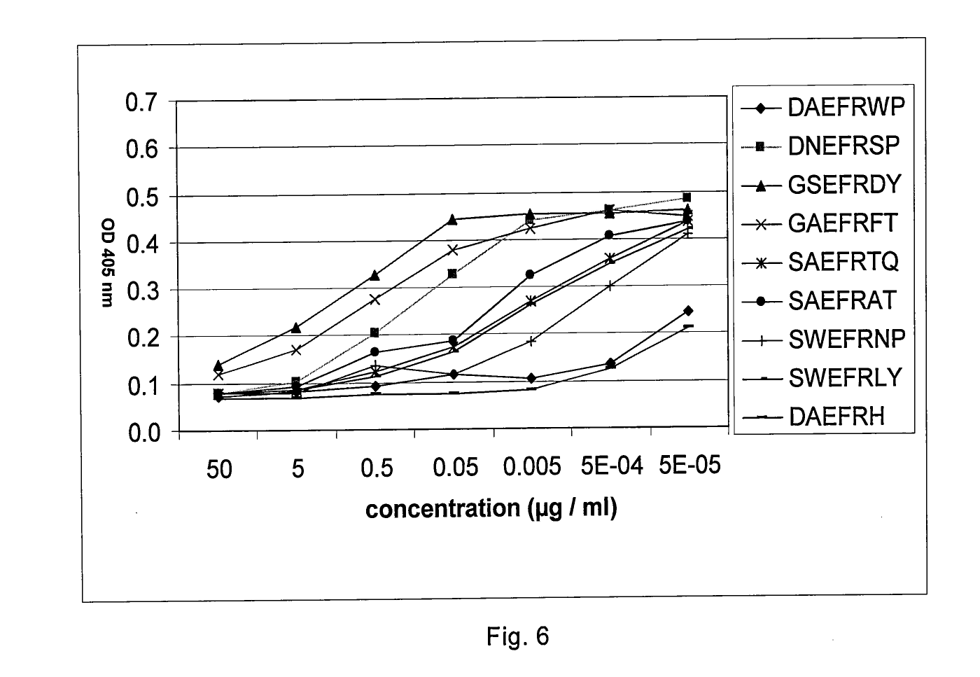

As expected and seen in FIG. 6 (showing peptides 4011-4018), peptide 1737 DAEFRH can compete with BSA-coupled, plate-bound peptide DAEFRH and thus inhibits recognition by the monoclonal antibody. Furthermore, it is shown that peptides 4012 DNEFRSP, 4013 GSEFRDY, and 4014 GAEFRFT are able to moderately inhibit binding of the monoclonal antibody to the original epitope. In contrast, peptides 4011 DAEFRWP, 4015 SAEFRTQ, 4016 SAEFRAT, 4017 SWEFRNP, and 4018 SWEFRLY (to a different extent) strongly block epitope recognition.

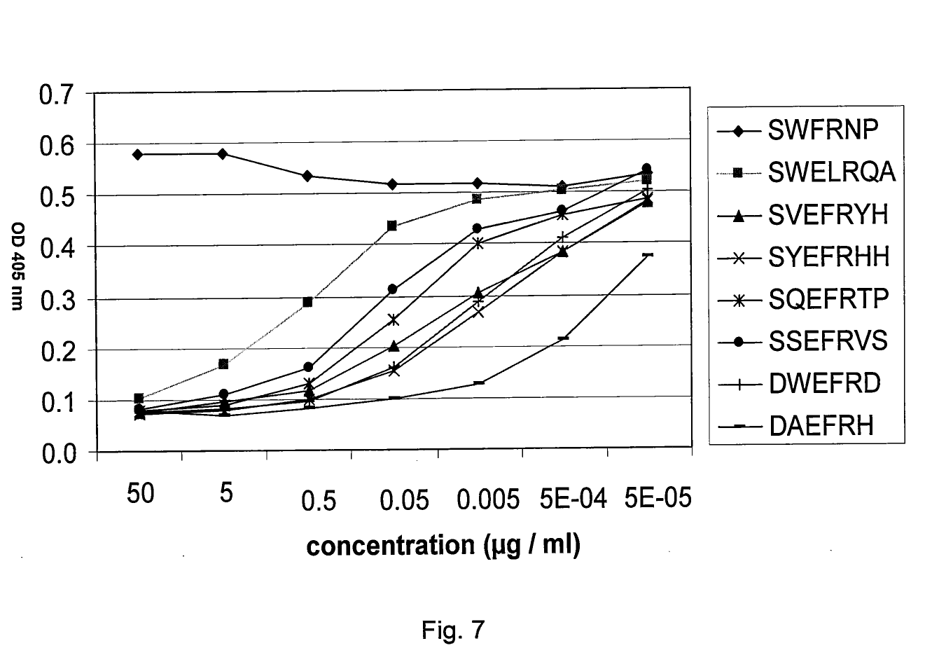

As expected and presented in FIG. 7 (showing peptides 4019-4025), peptide 1737 DAEFRH can successfully compete with BSA-coupled, plate-bound peptide DAEFRH for monoclonal antibody recognition in an additionally performed, independent experiment. Furthermore, it is shown that peptide 4019 SWFRNP is not inhibitory at the concentrations tested, whereas peptides 4020 SWELRQA, 4021 SVEFRYH, 4022 SYEFRHH, 4023 SQEFRTP, 4024 SSERFVS and 4025 DWEFRD (to a different extent) block epitope recognition. Peptides 4021, 4022, 4023, 4024 and 4025 are strong inhibitors with an IC50 of less than 0.5 μg/ml, whereas peptide 4020 is an intermediate inhibitor with an IC50 of more than 0.5 μg/ml.

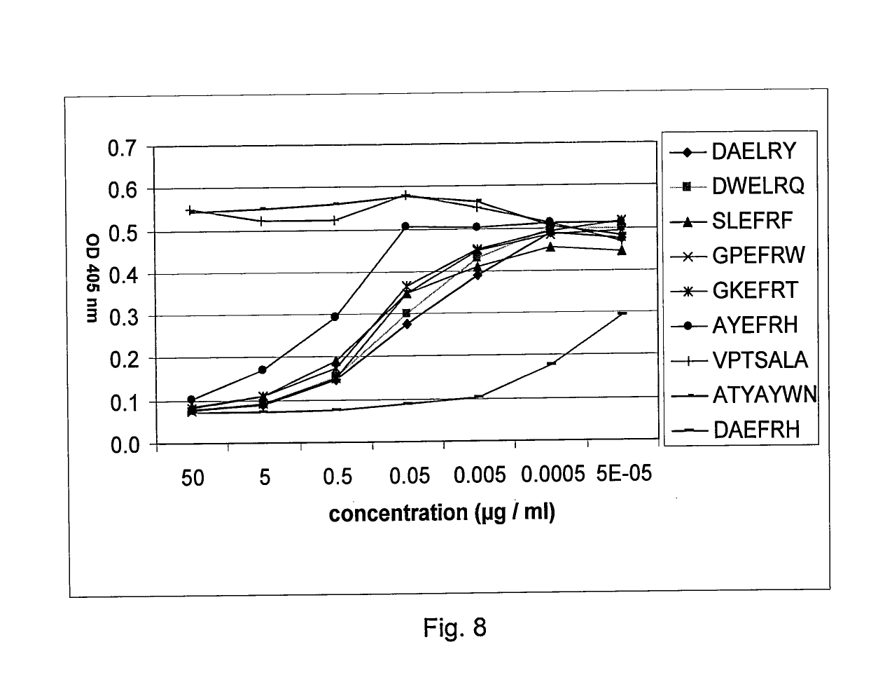

As expected and seen in FIG. 8 (peptides 4031-4038), peptide 1737 DAEFRH can successfully compete with BSA-coupled, plate-bound peptide DAEFRH for monoclonal antibody recognition in a 3rd independent experiment. Furthermore, it is shown that peptides 4037 VPTSALA and 4038 ATYAYWN are not inhibitory at the concentrations tested, whereas peptides 4031 DAELRY, 4032 DWELRQ, 4033 SLEFRF, 4034 GPEFRW, 4035 GKEFRT and 4036 AYEFRH (to a different extent) block epitope recognition. Peptides 4031, 4032, 4033, 4034 and 4035 are relatively strong inhibitors with an IC50 of less than 0.5 μg/ml, whereas peptide 4036 is a (relatively) weak inhibitor with an IC50 of more than 0.5 μg/ml.

In the following Table further examples of the immune response elicited by using AD mimotopes are described. All peptides listed in table 1 mount specific immune reactions against full length Aβ and/or fragments thereof.

| Internal Peptide number | Detection of Aβ |
|---|---|
| p1122 | + |
| p1123 | + |
| p1125 | + |
| p1238 | + |
| p1239 | + |
| p1252 | + |
| p1283 | + |
| p3005 | + |
| p3006 | + |
| p3007 | + |
| p3008 | + |
| p4003 | + |
| p4020 | + |
| p4023 | + |
| p4033 | + |
| p4034 | + |
| p4035 | + |

### Example 5

### Inhibition Assay with Defined 5Mer Peptides: Non-Natural Amino Acids

It has been shown previously that the 5mer peptide 1238 DKELR may be used as epitope in a mimotope-based Alzheimer vaccine (see PCT/EP04/00162). In the following, amino acids of the original 5mer epitope are replaced by non-natural amino acids: L is replaced by the non-natural amino acids tBuGly, Nle, Nva, or Cha.

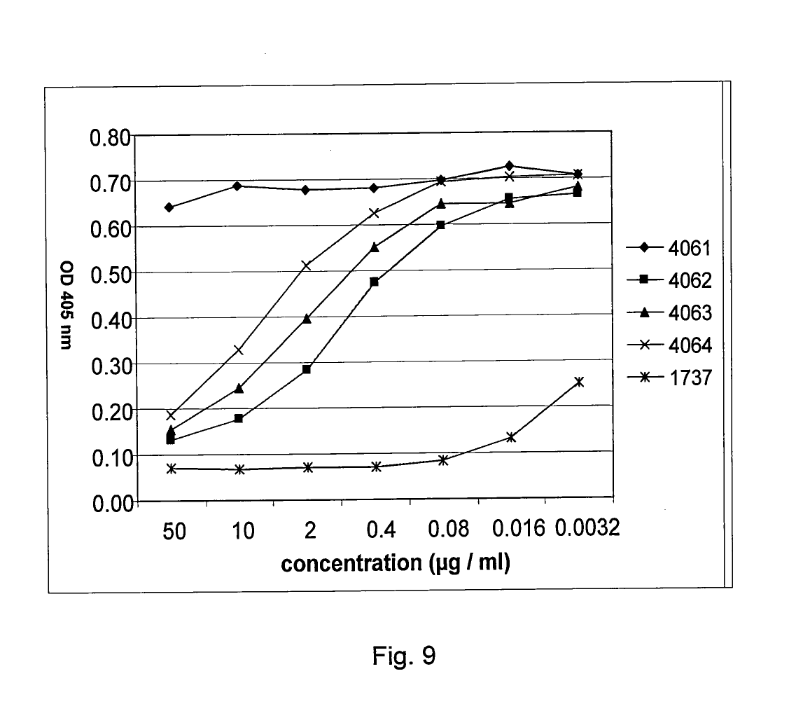

As expected and presented in FIG. 9 (peptides 4061-4064 DKELR variants), peptide 1737 DAEFRH can successfully compete with BSA-coupled, plate-bound peptide DAEFRH for monoclonal antibody recognition in a 4th independent experiment. Furthermore, it is shown that peptide 4061 DKE(tBuGly)R is not inhibitory at the concentrations tested. Interestingly, peptides 4062 DKE(Nle)R, 4063 DKE((Nva)R, and 4064 DKE(Cha)R (to a different extent) block epitope recognition. Peptides 4062, 4063, and 4064 are relatively weak inhibitors with an IC50 of more than 0.5 μg/ml.

### Example 6

### Generation of Monoclonal Antibodies to Specifically Detect β-Amyloid and N-Terminally Truncated and/or Post-translationally Modified β-Amyloid Fragments

Methods

The antibodies used for the mimotope identification according to the following examples detect amino acid sequences derived from human Aβ but do not bind to full length human APP. The sequences detected include EFRHDS (=Original epitope aa3-8 of Aβ), p(E)FRHDS (=Original epitope of the modified aa3-8 of Aβ), EVHHQK (=Original epitope aa11-16 of Aβ). The antibody may be a monoclonal or polyclonal antibody preparation or any anti-body part or derivative thereof, the only prerequisite is that the antibody molecule specifically recognises at least one of the epitopes mentioned above (derived from human Aβ), but does not bind to full length human APP.

The mimotopes are identified and further characterised with such monoclonal antibodies and peptide libraries.

### Example 6a

### Generation of Monoclonal Antibody MV-001

A monoclonal antibody derived from the fusion of experiment Alz-5 was generated: In experiment Alz-5 C57/B16 mice were immunized repeatedly with original Aβ epitope DAEFRHDSGYC coupled to KLH (Keyhole Limpet Hemocyanin) and Alum (Aluminium Hydroxyide) as adjuvant. p4371-peptide-specific, antibody-producing hybridomas were detected by ELISA (p1253- and p4371-peptide-coated ELISA plates). Human Aβ40/42 (recombinant protein) was used as positive control peptide: hybridomas recognizing the recombinant protein immobilised on ELISA plates were included because they are binding both peptide and full length Aβ specifically. P1454 (Human Aβ 33-40) was used as negative control peptide. Furthermore hybridomas were tested against p4373. Only hybridomas with no or limited p4373 binding were used for further antibody development.

The Hybridoma clone (MV-001 (internal name 824; IgG1) was purified and analysed for specific detection of p1253, p4371, p4373, p1454 and Aβ respectively. MV-001 recognized the injected epitope (p1253) as well as the specific epitope (p4371) and full length Aβ protein (recombinant protein; obtained from Bachem AG, Bubendorf, Switzerland) in ELISA. It however did not detect p1454 in ELISA. Furthermore, the MV-001 antibodies basically failed to detect the peptide p4373 encoding the pyroglutamate version of Aβ3-10 (30 times lower titer than the original epitopes).

### Example 6b

### Generation of Monoclonal Antibody MV-003

A monoclonal antibody derived from the fusion of experiment Alz-16 was generated: In experiment Alz-16 BalbC mice were immunized repeatedly with the epitope p(E)FRHDSC (p4373) coupled to KLH (Keyhole Limpet Hemocyanin) and Alum (Aluiminium Hydroxyide) as adjuvant. p4373-peptide-specific, antibody-producing hybridomas were detected by ELISA (p4373-peptide-coated ELISA plates). p1253, p1454 and Aβ40/42 were used as negative control peptides. Furthermore, hybridomas were tested against p4371. Only hybridomas with no or limited p4371 binding were used for further antibody development in order to guarantee for pyroglutamate-specificity.

The Hybridoma clone (MV-003 (internal name D129; IgG1) was purified and analysed for specific detection of p1253, p4371, p4373, p1454 and Aβ respectively. MV-003 recognized the injected epitope (p4373) but failed to detect p1454, p1253 or full length Aβ protein (recombinant protein; obtained from Bachem AG, Bubendorf, Switzerland) in ELISA. Furthermore, the MV-003 antibodies failed to detect the peptide p4371 encoding the normal version of Aβ3-10 (15 times lower titer than the original epitope).

### Example 6c

### Generation of Monoclonal Antibody MV-004

A monoclonal antibody derived from the fusion of experiment Alz-15 was generated: In experiment Alz-15 BalbC mice were immunized repeatedly with the epitope EVHHQKC (p4372) coupled to KLH (Keyhole Limpet Hemocyanin) and Alum (Aluiminium Hydroxide) as adjuvant. p4372-peptide-specific, antibody-producing hybridomas were detected by ELISA (p4372-peptide-coated ELISA plates). P4376, p4378, p1454 and Aβ40/42 were used as negative control peptides. Only hybridomas with no or limited p4376 and p4378 binding were used for further antibody development in order to guarantee for specificity against the free N-Terminus at position aa11.

The Hybridoma clone (MV-004 (internal name B204; IgG1) was purified and analysed for specific detection of p4372, p4376, p4378, p1454 and Aβ respectively. MV-004 recognized the injected epitope (p4372) but failed to detect p1454, p4376 and p4378 as well as full length Aβ protein (recombinant protein; obtained from Bachem AG, Bubendorf, Switzerland) in ELISA. The failure to detect p4376, p4378 demonstrates specificity for the free N-terminus at position aa11 in truncated Aβ.

### Example 6d

### Generation of Monoclonal Antibodies to Specifically Detect β-Amyloid and N-Terminally Truncated and/or Post-translationally Modified β-Amyloid Fragments-Monoclonal Anti-Body MV-002

Methods

The antibodies used for the mimotope identification according to the present invention detect amino acid sequences derived from human Aβ but do not bind to full length human APP. The sequences detected include EVHHQKLVFFAED (=Original epitope aa11-24 of Aβ) and p(E)VHHQKLVF (p4374=original epitope aa11-19 of Aβ with a pyroglutamate modification at the N-Terminus). The antibody may be a monoclonal or polyclonal antibody preparation or any antibody part or derivative thereof, the only prerequisite is that the antibody molecule specifically recognises at least one of the epitopes mentioned above (derived from human Aβ), but does not bind to full length human APP.

The mimotopes are identified and further characterised with such monoclonal antibodies and peptide libraries.

A monoclonal antibody derived from the fusion of experiment Alz-9 was generated: C57/B16 mice were immunized repeatedly with original Aβ epitope HQKLVFC coupled to KLH (Keyhole Limpet Hemocyanin) and Alum (Aluiminium Hydroxide) as adjuvant. p4377 peptide-specific, antibody-producing hybridomas were detected by ELISA (p4377-peptide-coated ELISA plates). Human Aβ40/42 (recombinant protein) was used as positive control peptide: hybridomas recognizing the recombinant protein immobilised on ELISA plates were included because they were binding both peptide and full length Aβ specifically. p1454 (Human Aβ 33-40) was used as negative control peptide. Furthermore hybridomas were tested against p4374, p1323 and sAPP-alpha. Only hybridomas with good p4374, and p1323 binding and a lack of sAPP-alpha binding were used for further antibody development.

The Hybridoma clone MV-002 (internal name A115; IgG2b) was purified and analysed for specific detection of p1323, p4374, p4377, p1454, Aβ and sAPP-alpha respectively. MV-002 recognized the epitopes p1323 as well as p4377 and full length Aβ protein (recombinant protein; obtained from Bachem AG, Bubendorf, Switzerland) in ELISA. It however did not detect p1454 in ELISA. Furthermore, the MV-002 antibodies failed to detect sAPP-alpha but bound specifically to the peptide p4374 encoding the pyroglutamate version of Aβ11-19.

### Example 7

### Phage Display, In Vitro Binding and Inhibition ELISA

Phage Display libraries used in this example were: Ph.D. 7: New England BioLabs E8102L (linear 7mer library). Phage Display was done according to manufacturer's protocol (www.neb.com).

After 2 or 3 subsequent rounds of panning, single phage clones were picked and phage supernatants were subjected to ELISA on plates coated with the antibody that was used for the panning procedure. Phage clones that were positive in this ELISA (strong signal for the target, but no signal for unspecific control) were sequenced. From DNA sequences, peptide sequences were deduced. These peptides were synthesized and characterised in binding and inhibition ELISA. Additionally, some novel mimotopes were created by combining sequence information from mimotopes identified in the screen to support the identification of a consensus sequence for a mimotope vaccination.

1. In Vitro Binding Assay (ELISA)

Peptides derived from Phage Display as well as variants thereof were coupled to BSA and bound to ELISA plates (1 μM; as indicated in the respective figures) and subsequently incubated with the monoclonal antibody that was used for the screening procedure to analyse binding capacity of identified peptides.

2. In Vitro Inhibition Assay (ELISA)

Different amounts of peptides (concentrations ranging from 10 μg to 0.08 μg; serial dilutions; for MV-002: concentrations ranging from 5 μg to 0.03 μg; serial dilutions), derived from Phage Display were incubated with the monoclonal antibody that was used for the screening procedure. Peptides diminishing subsequent binding of the antibody to the original epitope coated on ELISA plates were considered as inhibiting in this assay.

### Example 8

### In Vivo Testing of Mimotopes: Analysis of Immunogenicity and Crossreactivity

1. In Vivo Testing of Mimotopes

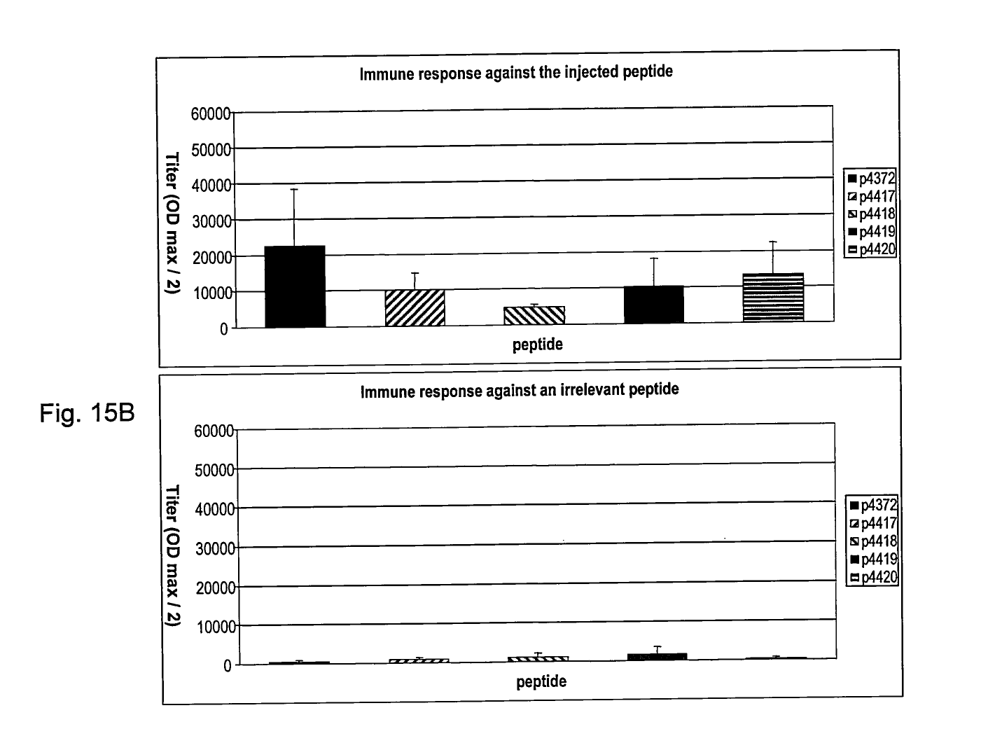

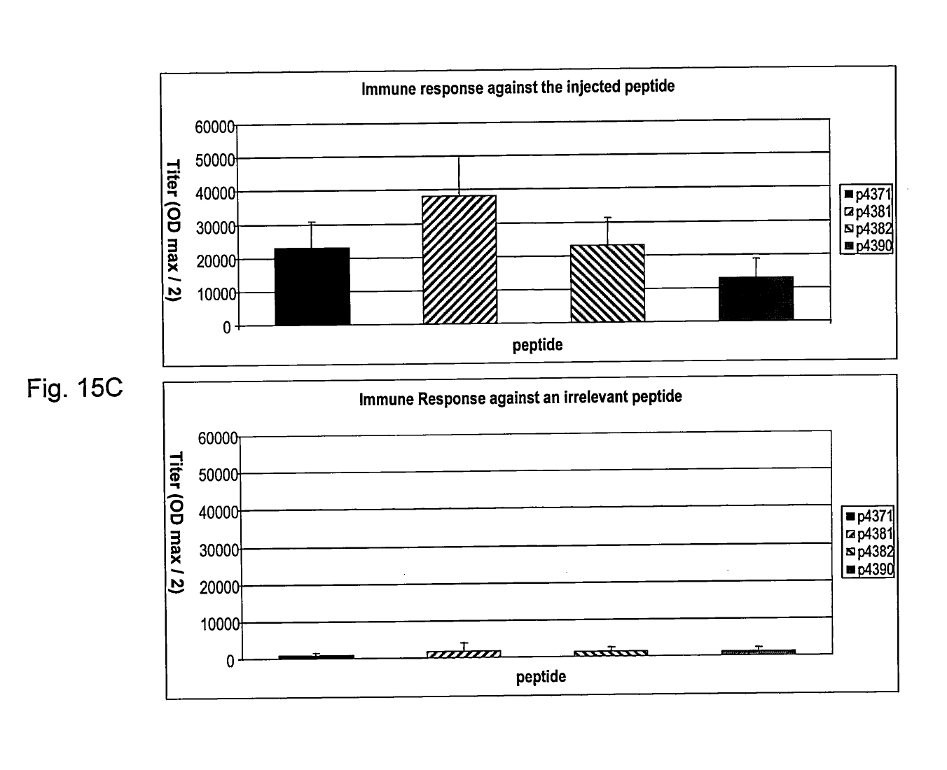

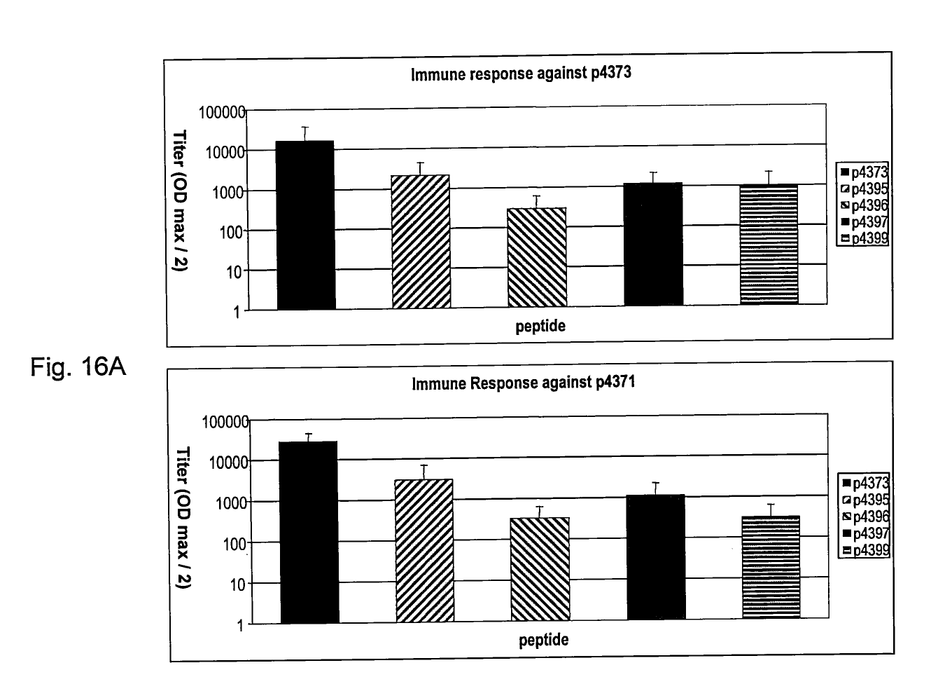

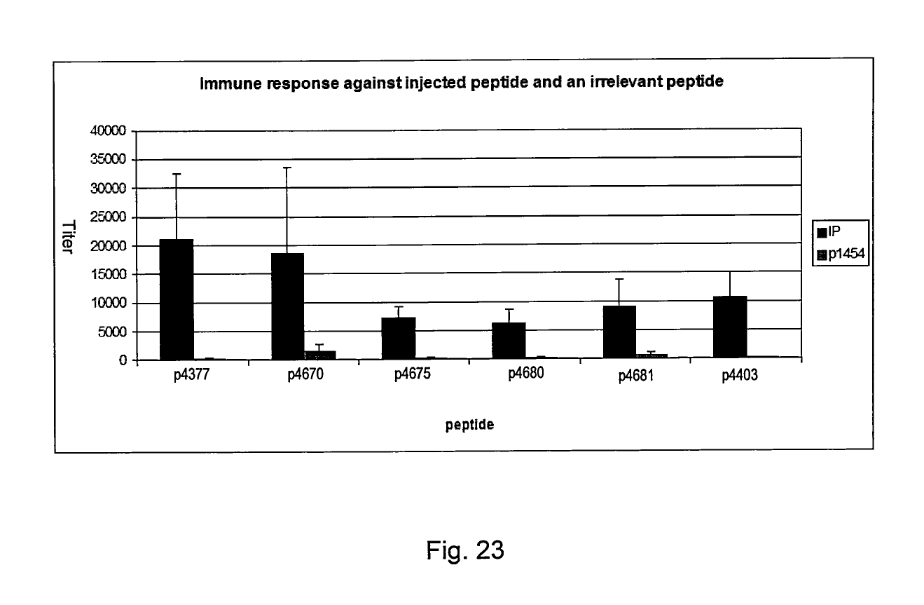

Inhibiting as well as non-inhibiting peptides were coupled to KLH and injected into mice (wildtype C57/B16 mice; subcutaneous injection into the flank) together with an appropriate adjuvant (aluminium hydroxide). Animals were vaccinated 3-6 times in biweekly intervals and sera were taken biweekly as well. Titers to injected peptides, as well as to an irrelevant peptide were determined with every serum. Furthermore, titers against the recombinant human Aβ protein, and against original peptides were determined respectively. In general sera were analysed by reaction against peptides coupled to Bovine Serum Albumin (BSA) and recombinant full length proteins which were immobilised on ELISA plates. Titers were determined using anti mouse IgG specific antibodies. For detailed results see FIGS. 15, 16 and 17 respectively and FIGS. 23, 24 and 25 respectively.

2. Results for MV-001, MV-003 and MV-004

2.1. Identification of Specific Monoclonal Antibodies (mAB) Directed Against n-Terminally Truncated and Modified Forms of Aβ:

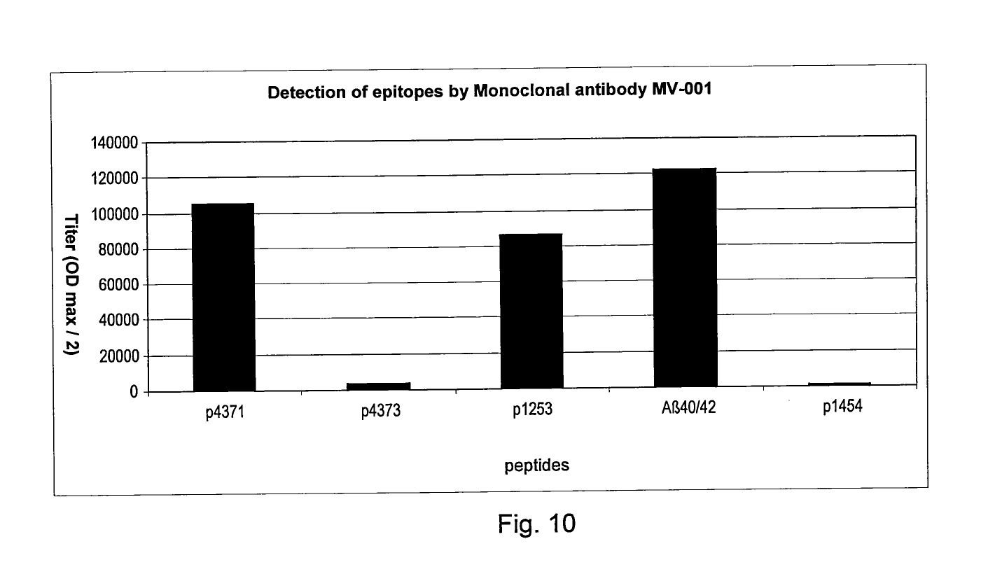

FIG. 10 depicts the characterisation of the monoclonal anti-body MV-001 (internal name 824; IgG1) derived from experiment Alz-5 demonstrating specificity for full length Aβ and Aβ truncated at position E3.

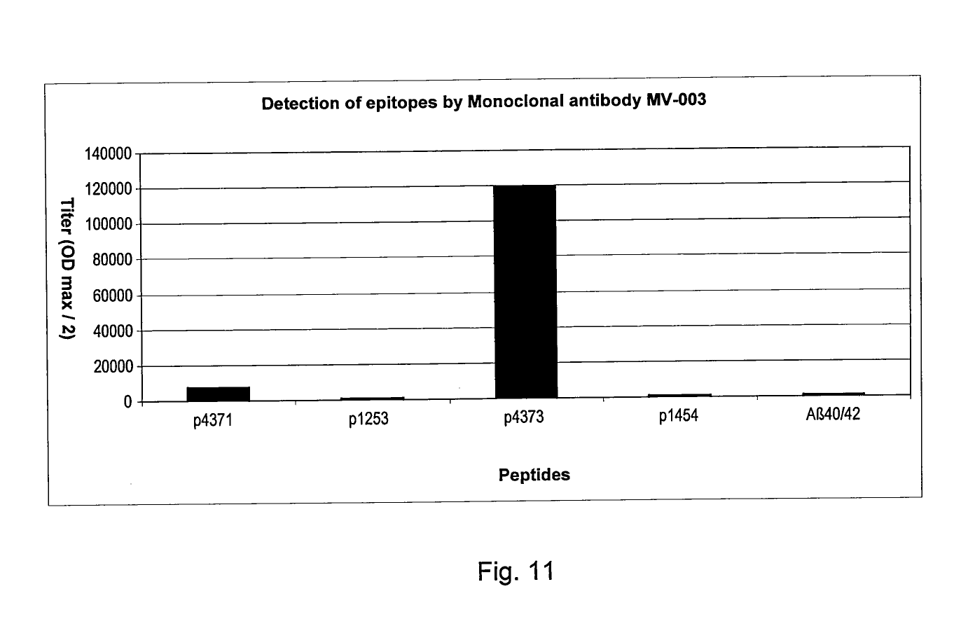

FIG. 11 depicts the characterisation of the monoclonal anti-body MV-003 (internal name D129; IgG1) derived from experiment Alz-16 demonstrating specificity for Aβ truncated and posttrans-lationally modified at position p(E)3.

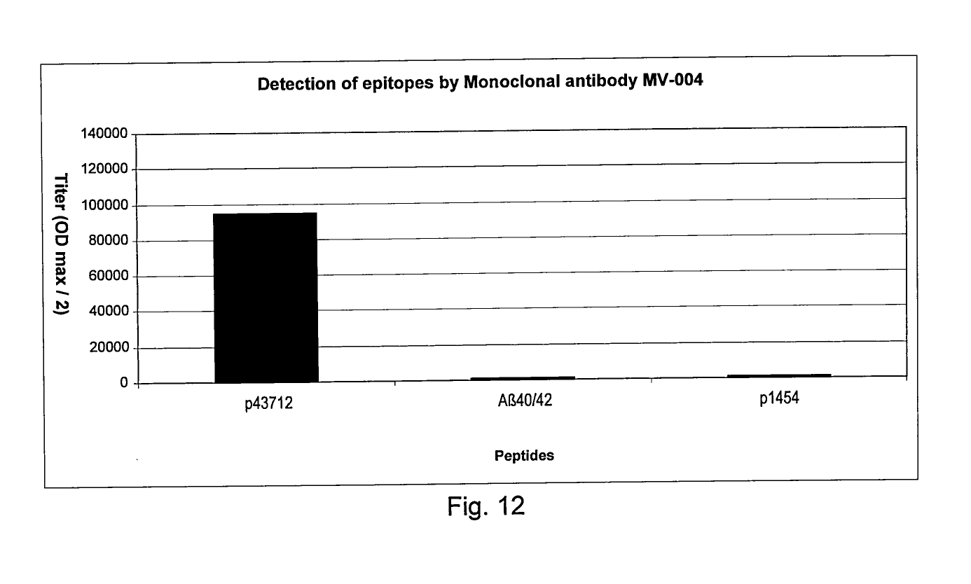

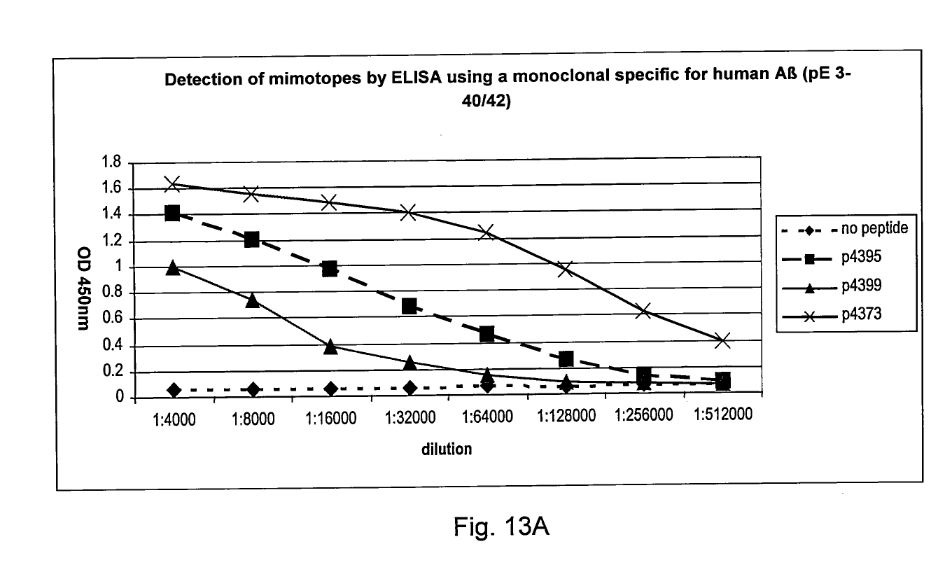

FIG. 12 depicts the characterisation of the monoclonal anti-body MV-004 (internal name B204; IgG1) derived from experiment Alz-15 demonstrating specificity for Aβ truncated at position E11.

2.2. Screening with Specific mABs Directed Against n-Terminally Truncated and Modified Forms of Aβ:

2.2.1. Phage Display Library Ph.D. 7

2.2.1.1. Screening with Monoclonal Antibody Directed Against p4373

8 Sequences were identified by screening PhD 7 phage display libraries in this screen: Table 1A summarises the peptides identified and their binding capacity as compared to the original epitope.

2.2.1.2. Screening with Monoclonal Antibody Directed Against p4372

9 Sequences were identified by screening PhD 7 phage display libraries in this screen: Table 1B summarises the peptides identified and their binding capacity as compared to the original epitope.

2.2.1.3. Screening with Monoclonal Antibody Directed Against p4371

71 Sequences were identified by screening PhD 7 and PhD12 phage display libraries in this screen: Table 1C summarises the peptides identified and their binding capacity as compared to the original epitope.

TABLE 1A
mimotopes binding to the
parental antibody MV-003

| Internal | | |
|---|---|---|
| Peptide | | Binding |
| number | Sequence | Capacity |
| p4395 | IRWDTPC | 2 |
| p4396 | VRWDVYPC | 1 |
| p4397 | IRYDAPLC | 1 |
| p4399 | IRYDMAGC | 1 |
| p4728 | IRWDTSLC | 3 |
| p4756 | IRWDQPC | 3 |
| p4792 | IRWDGC | 1 |
| p4793 | IRWDGGC | 2 |

Legend to Table 1A: the binding capacity is coded by the following binding code: 1:X describes the dilution factor of the parental AB.

| | | OD halfmax |
|---|---|---|
| binding code | | 1:X |
| 0 | no binding | :0 |
| 1 | weak binding | :<16000 |
| 2 | medium binding | :16-60000 |
| 3 | strong binding | :>60000 |

TABLE 1B
mimotopes binding to the
parental antibody MV-004

| Internal | | |
|---|---|---|
| Peptide | | Binding |
| number | Sequence | Capacity |
| p4417 | EVWHRHQC | 2 |
| p4418 | ERWHEKHC | 3 |
| p4419 | EVWHRLQC | 3 |
| p4420 | ELWHRYPC | 2 |
| p4665 | ELWHRAFC | 2 |
| p4786 | ELWHRAC | 1 |
| p4788 | EVWHRGC | 1 |
| p4789 | EVWHRHC | 1 |
| p4790 | ERWHEKC | 1 |

Legend to Table 1B: the binding capacity is coded by the following binding code: 1:X describes the dilution factor of the parental AB.

| binding code | | OD halfmax 1:X |
|---|---|---|
| 0 | no binding | :0 |
| 1 | weak binding | :<24000 |
| 2 | medium binding | :24-96000 |
| 3 | strong binding | :>96000 |

TABLE 1C
mimotopes binding to the
parental antibody MV-001

| Internal | | |
|---|---|---|
| Peptide | | Binding |
| number | Sequence | Capacity |
| p4380 | QDFRHYC | 2 |
| p4381 | SEFKHGC | 3 |
| p4382 | TSFRHGC | 2 |
| p4383 | TSVFRHC | 3 |
| p4384 | TPFRHTC | 2 |
| p4385 | SQFRHYC | 2 |
| p4386 | LMFRHNC | 3 |
| p4387 | SAFRHHC | 2 |
| p4388 | LPFRHGC | 2 |
| p4389 | SHFRHGC | 2 |
| p4390 | ILFRHGC | 3 |
| p4391 | QFKHDLC | 2 |
| p4392 | NWFPHPC | 1 |
| p4393 | EEFKYSC | 2 |
| p4701 | NELRHSTC | 3 |
| p4702 | GEMRHQPC | 3 |
| p4703 | DTYFPRSC | 2 |
| p4704 | VELRHSRC | 2 |
| p4705 | YSMRHDAC | 2 |
| p4706 | AANYFPRC | 2 |
| p4707 | SPNQFRHC | 3 |
| p4708 | SSSFFPRC | 2 |
| p4709 | EDWFFWHC | 1 |
| p4710 | SAGSFRHC | 3 |
| p4711 | QVMRHHAC | 2 |
| p4712 | SEFSHSSC | 3 |
| p4713 | QPNLFYHC | 1 |
| p4714 | ELFKHHLC | 3 |
| p4715 | TLHEFRHC | 3 |
| p4716 | ATFRHSPC | 2 |
| p4717 | APMYFPHC | 2 |
| p4718 | TYFSHSLC | 2 |
| p4719 | HEPLFSHC | 1 |
| p4721 | SLMRHSSC | 2 |
| p4722 | EFLRHTLC | 3 |
| p4723 | ATPLFRHC | 3 |
| p4724 | QELKRYYC | 1 |
| p4725 | THTDFRHC | 3 |
| p4726 | LHIPFRHC | 3 |
| p4727 | NELFKHFC | 2 |
| p4729 | SQYFPRPC | 2 |
| p4730 | DEHPFRHC | 3 |
| p4731 | MLPFRHGC | 2 |
| p4732 | SAMRHSLC | 2 |
| p4733 | TPLMFWHC | 1 |
| p4734 | LQFKHSTC | 2 |
| p4735 | ATFRHSTC | 2 |
| p4736 | TGLMFKHC | 2 |
| p4737 | AEFSHWHC | 2 |
| p4738 | QSEFKHWC | 3 |
| p4739 | AEFMHSVC | 2 |
| p4740 | ADHDFRHC | 3 |
| p4741 | DGLLFKHC | 3 |
| p4742 | IGFRHDSC | 2 |
| p4743 | SNSEFRRC | 3 |
| p4744 | SELRHSTC | 3 |
| p4745 | THMEFRRC | 3 |
| p4746 | EELRHSVC | 3 |
| p4747 | QLFKHSPC | 3 |
| p4748 | YEFRHAQC | 3 |
| p4749 | SNFRHSVC | 3 |
| p4750 | APIQFRHC | 3 |
| p4751 | AYFPHTSC | 2 |
| p4752 | NSSELRHC | 3 |
| p4753 | TEFRHKAC | 3 |
| p4754 | TSTEMWHC | 1 |
| p4755 | SQSYFKHC | 3 |
| p4800 | CSEFKH | 3 |
| p4801 | SEFKHC | 3 |
| p4802 | CHEFRH | 3 |
| p4803 | HEFRHC | 3 |

Legend to Table 1C: the binding capacity is coded by the following binding code: 1:X describes the dilution factor of the parental AB

| binding code | | OD halfmax 1:X |
|---|---|---|
| 0 | no binding | :0 |
| 1 | weak binding | :<4000 |
| 2 | medium binding | :4000-20000 |
| 3 | strong binding | :>20000 |

2.3. In Vitro Characterisation of Mimotopes Identified in Screening Phage Display Libraries with Monoclonal Antibodies Directed Against n-Terminally Truncated and Modified Forms of Aβ:

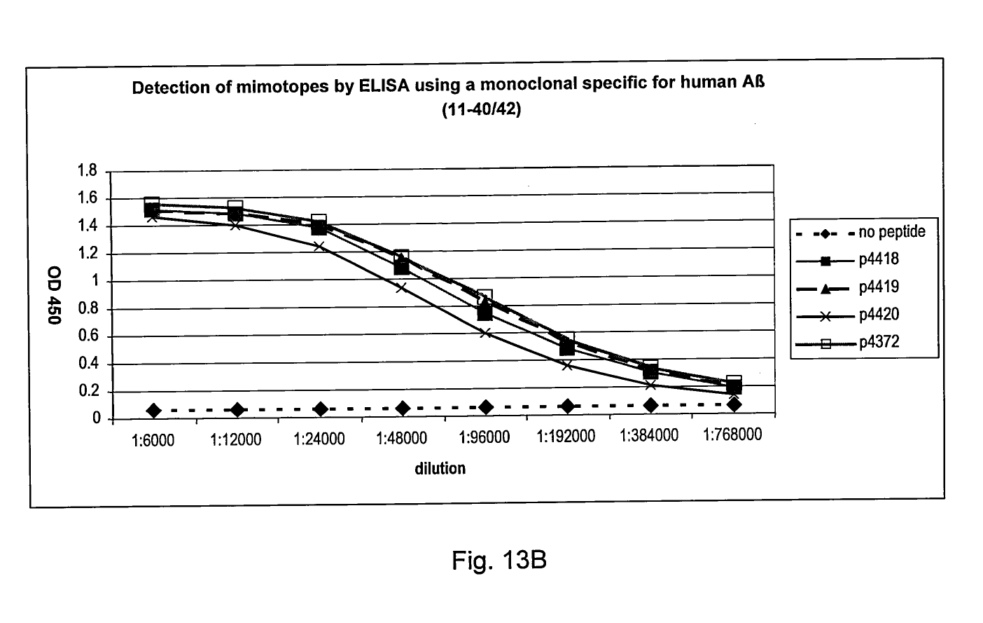

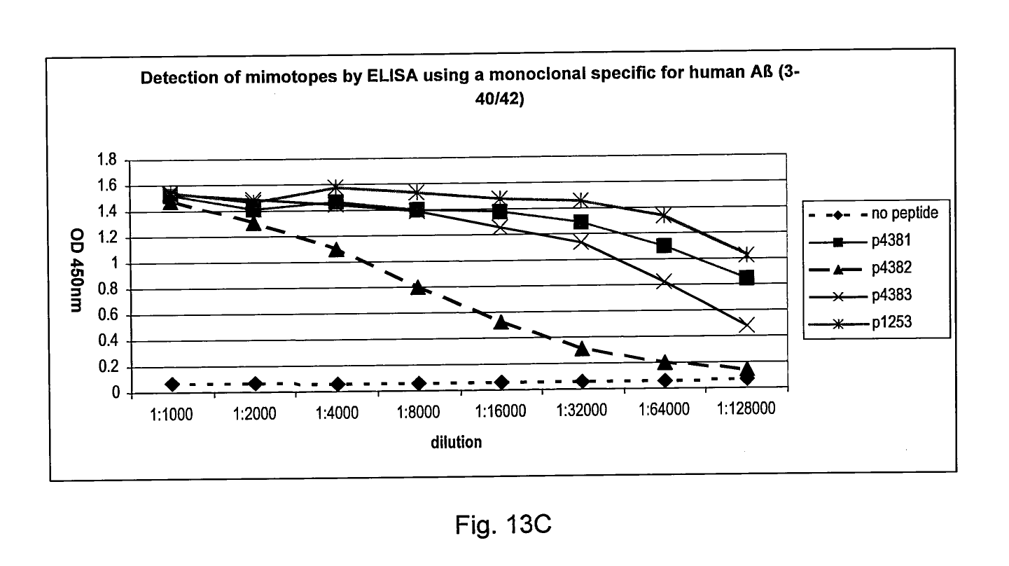

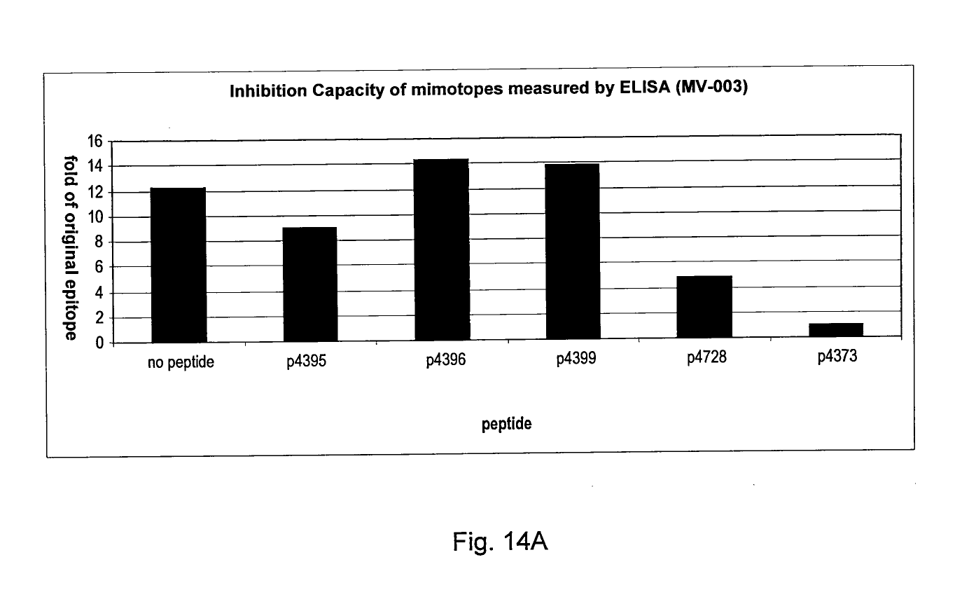

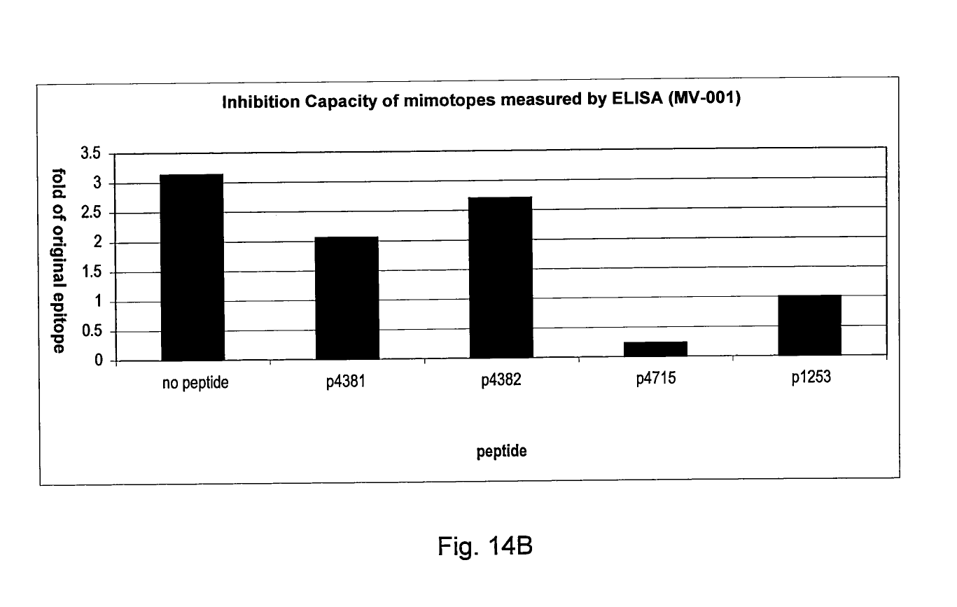

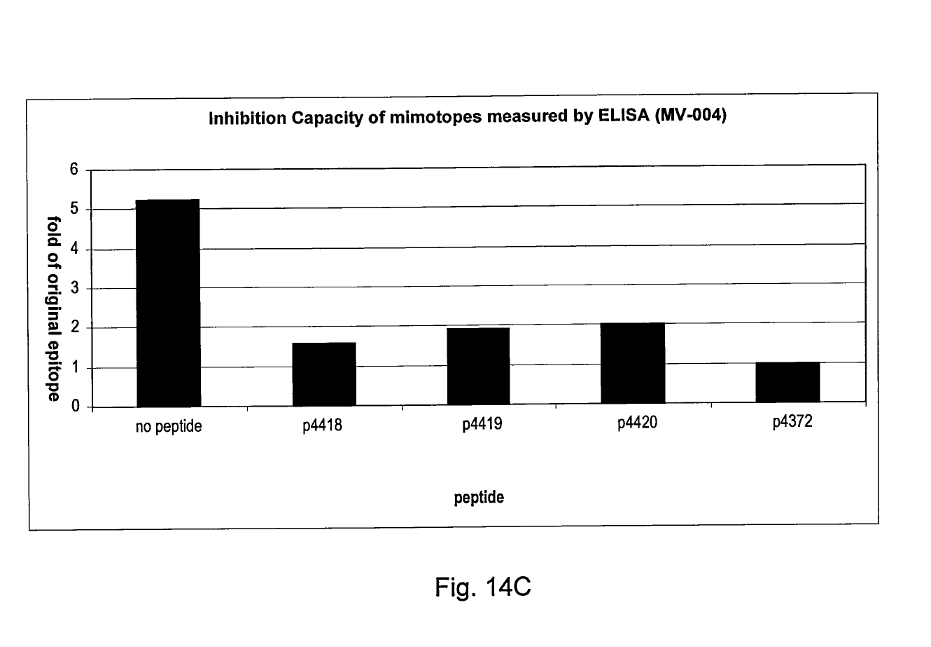

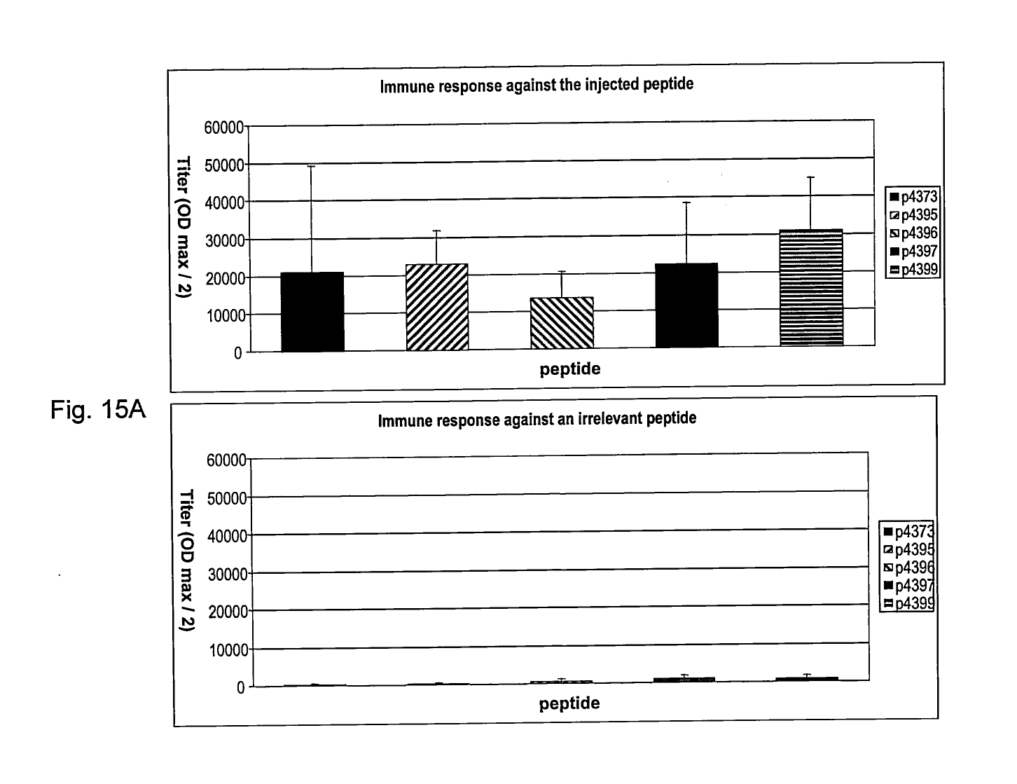

FIGS. 13 and 14 show representative examples for binding and inhibition assays used to characterise mimotopes in vitro. Data obtained are summarised in Tables 1 and 2 respectively.

MV-003 Mimotopes: From the 8 sequences presented 6 sequences inhibit binding of the p(E)3-7Aβ specific monoclonal antibody in in vitro competition experiments: Additional 2 sequences were identified that do not inhibit binding of monoclonal antibody in in vitro competition experiments but still retain binding capacity to the parental antibody (Table 2A).

MV-004 Mimotopes: All the 9 sequences presented inhibit binding of the monoclonal antibody specifically binding the free N-terminus of Aβ truncated at position E11 in in vitro competition experiments: (Table 2B).

MV-001 Mimotopes: From the 71 sequences presented 27 sequences inhibit binding of the monoclonal antibody specifically directed against Aβ truncated at position E3 in in vitro competition experiments: Additional 44 sequences were identified that do not inhibit binding of monoclonal antibody in in vitro competition experiments but still retain binding capacity to the parental antibody (Table 2C).

Table 2: mimotopes identified in this invention giving positive results in inhibiting assays

TABLE 2A
MV-003 Mimotopes

| Internal | | |
|---|---|---|
| Peptide | | Inhibition |
| number | Sequence | Capacity |
| p4395 | IRWDTPC | 1 |
| p4397 | IRYDAPLC | 1 |
| p4728 | IRWDTSLC | 2 |
| p4756 | IRWDQPC | 1 |
| p4792 | IRWDGC | 1 |
| p4793 | IRWDGGC | 1 |

Legend to Table 2A: the inhibition capacity is coded by the following code:
Weak inhibition means more peptide is required to lower AB binding than with the original epitope; strong inhibition means similar peptide amounts are required for mimotope and original epitope for lowering AB binding. Mimotopes are compared to the original peptide as standard. OD at 10 ug peptide used in the assay is used to calculate the competition capacity compared to original peptide.

| competition | |
|---|---|
| code | |
| 0 | no inhibition (OD of 10 ug peptide above 12 times |
| | of original peptide) |
| 1 | Weaker than original epitope (OD of 10 ug peptide |
| | below 12 times of original peptide) |
| 2 | strong inhibition (as original epitope; OD of |
| | 10 ug peptide below 5 times of original peptide) |

TABLE 2B
MV-004 Mimotopes

| Internal | | |
|---|---|---|
| Peptide | | Inhibition |
| number | Sequence | Capacity |
| p4417 | EVWHRHQC | 1 |
| p4418 | ERWHEKHC | 2 |
| p4419 | EVWHRLQC | 2 |
| p4420 | ELWHRYPC | 1 |
| p4665 | ELWHRAFC | 2 |
| p4786 | ELWHRAC | 1 |
| p4788 | EVWHRGC | 1 |
| p4789 | EVWHRHC | 1 |
| p4790 | ERWHEKC | 2 |

Legend to Table 2B: the inhibition capacity is coded by the following code:
Weak inhibition means more peptide is required to lower AB binding than with the original epitope; strong inhibition means similar peptide amounts are required for mimotope and original epitope for lowering AB binding. Mimotopes are compared to the original peptide as standard. OD at 10 ug peptide used in the assay is used to calculate the competition capacity compared to original peptide.

| competition | |
|---|---|
| code | |
| 0 | no inhibition (OD of 10 ug peptide above 5 times |
| | of original peptide) |
| 1 | Weaker than original epitope (OD of 10 ug peptide |
| | below 5 times of original peptide) |
| 2 | strong inhibition (as original epitope; OD of |
| | 10 ug peptide below 2 times of original peptide) |

TABLE 2C
MV-001 Mimotopes

| Internal | | |
|---|---|---|
| Peptide | | Inhibition |
| number | Sequence | Capacity |
| p4380 | QDFRHYC | 1 |
| p4381 | SEFKHGC | 1 |
| p4382 | TSFRHGC | 1 |
| p4383 | TSVFRHC | 1 |
| p4384 | TPFRHTC | 1 |
| p4385 | SQFRHYC | 1 |
| p4386 | LMFRHNC | 1 |
| p4387 | SAFRHHC | 1 |
| p4388 | LPFRHGC | 1 |
| p4389 | SHFRHGC | 1 |
| p4390 | ILFRHGC | 1 |
| p4391 | QFKHDLC | 1 |
| p4392 | NWFPHPC | 1 |
| p4393 | EEFKYSC | 1 |
| p4707 | SPNQFRHC | 1 |
| p4715 | TLHEFRHC | 2 |
| p4725 | THTDFRHC | 1 |
| p4730 | DEHPFRHC | 1 |
| p4738 | QSEFKHWC | 1 |
| p4740 | ADHDFRHC | 1 |
| p4741 | DGLLFKHC | 1 |
| p4746 | EELRHSVC | 1 |
| p4753 | TEFRHKAC | 2 |
| p4800 | CSEFKH | 2 |
| p4801 | SEFKHC | 1 |
| p4802 | CHEFRH | 2 |
| p4803 | HEFRHC | 2 |

Legend to Table 2C: the inhibition capacity is coded by the following code:
Weak inhibition means more peptide is required to lower AB binding than with the original epitope; strong inhibition means similar peptide amounts are required for mimotope and original epitope for lowering AB binding. Mimotopes are compared to the original peptide as standard. OD at 10 ug peptide used in the assay is used to calculate the competition capacity compared to original peptide.

| competition | |
|---|---|
| code | |
| 0 | no inhibition (OD of 10 ug peptide above 3 times |
| | of original peptide) |
| 1 | Weaker than original epitope (OD of 10 ug peptide |
| | below 3 times of original peptide) |
| 2 | strong inhibition (as original epitope; OD of |
| | 10 ug peptide below 2 times of original peptide) |

TABLE 3
Non-mimotope peptides

| Internal | |
|---|---|
| Peptide | |
| number | Sequence |
| p1253 | DAEFRHDSGYC |
| p4371 | EFRHDS-C |
| p4372 | EVHHQK-C |
| p4373 | p(E)FRHDS-C |
| p4374 | p(E)VHHQKLVFC |
| p4376 | GYEVHHQKC |
| p4377 | EVHHQKLVFC |
| p4378 | C-EVHHQKLVFF |
| p1454 | CGLMVGGVV |
| Aβ1-40 | DAEFRHDSGYEVHHQKLVFFAEDVGSNKGAIIGLMVGGVV |
| Aβ1-42 | DAEFRHDSGYEVHHQKLVFFAEDVGSNKGAIIGLMVGGVVIA |
| sAPPalpha | alpha-Secretase induced cleavage product |
| | derived from human APP (gi:112927) |

2.4. In Vivo Characterisation of Mimotopes Identified in Screening Phage Display Libraries with a Monoclonal Antibody Directed Against n-Terminally Truncated and Modified Forms of Aβ:

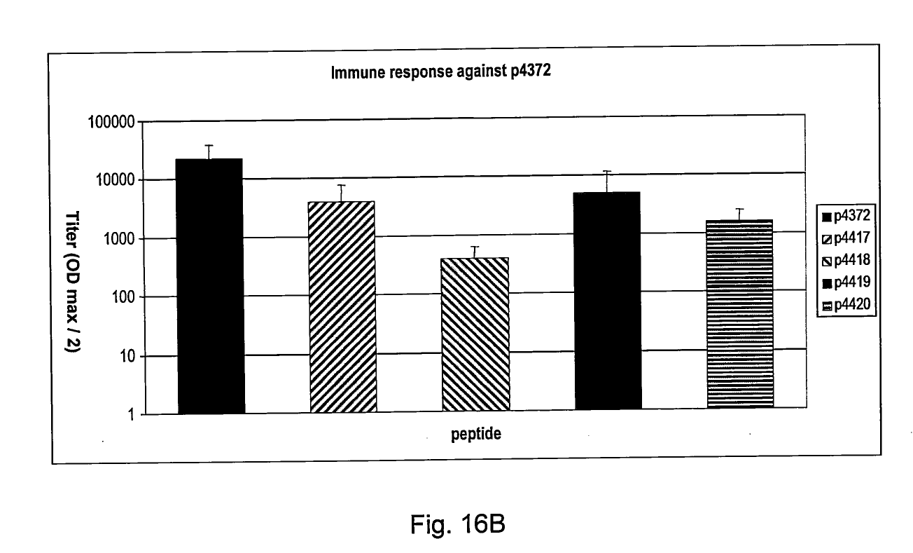

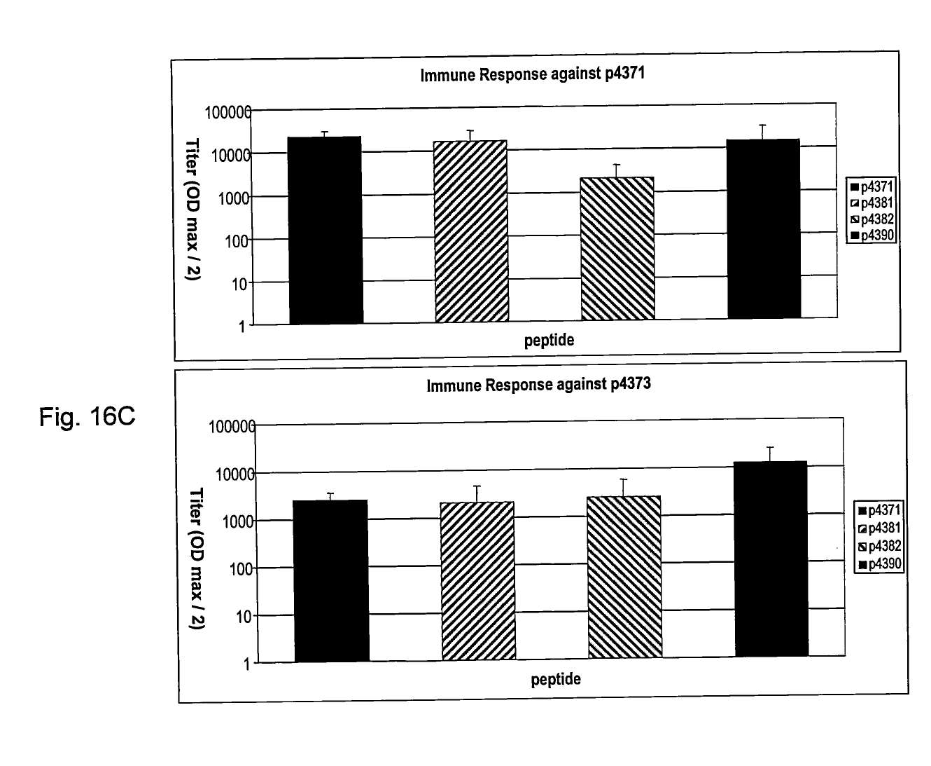

Female C57/b16 mice, 5-6 mice per group, were subcutaneously immunized with 30 μg peptide coupled to KLH. Control groups were administered original epitope-KLH conjugates respectively. As adjuvant alum was used (always 1 mg per mouse). The peptides administered were all able to bind to monoclonal antibodies specifically although some of the peptides did not inhibit the binding of the original epitope to its parental antibody in vitro (in an in vitro inhibition assay). The in vitro ELISA assay to determine the antibody titer was performed with sera of single mice after each vaccination in a two week interval (see FIGS. 15 and 16 respectively). The wells of the ELISA plate were coated with mimotope-BSA conjugate and an irrelevant peptide-BSA conjugate (negative control). The positive control was performed by reaction of the parental antibody with the respective mimotope-BSA conjugate. The detection was performed with anti-mouse IgG. Additionally, recombinant proteins were immobilised on ELISA plates and sera reacted accordingly. FIGS. 15 to 17 show representative examples for assays used to characterise mimotopes in vivo.

FIG. 15 shows examples for in vivo characterisations of the immune response elicited by mimotope vaccination by analysing the immune response against injected peptide and an irrelevant peptide, containing an unrelated sequence. In all three examples shown, the original epitopes and the mimotopes, elicit immune responses against the injected peptides but fail to induce a relevant immune response against an unrelated sequence (p1454).

As example for MV-003-mimotopes, original epitope p4373 and the mimotopes p4395, p4396, p4397, and p4399 are depicted in FIG. 15A. All vaccines are mounting similar immune responses against their respective mimotopes. Neither original epitope p4373-vaccine treated nor the animals treated with mimotope p4395, p4396, p4397 or p4399-vaccines mount relevant titers against irrelevant peptide p1454 (11×-25× less than injected peptides).

As example for MV-004-mimotopes original epitope p4372 and the mimotopes p4417, p4418, p4419, and p4420 are depicted in FIG. 15B. All vaccines are mounting similar immune responses against their respective mimotopes. Neither original epitope p4372-vaccine treated nor the animals treated with mimotope p4417, p4418, p4419, and p4420-vaccines mount relevant titers against irrelevant peptide p1454 (20-80× less than injected peptides).

As example for MV-001-mimotopes original epitope p4371 and the mimotopes p4381, p4382, and p4390 are depicted in FIG. 15C. All vaccines are mounting similar immune responses against their respective mimotopes. Neither original epitope p4371-vaccine treated nor the animals treated with mimotope p4381, p4382, and p4390-vaccines mount relevant titers against irrelevant peptide p1454 (>10× less than injected peptides).

FIG. 16 shows examples for in vivo characterisations of the immune response elicited by mimotope vaccination against the respective original epitope of the parental antibody as well as against peptides derived of other forms of truncated species of Aβ.

As example for MV-003-mimotopes, original epitope p4373 and the mimotopes p4395, p4396, p4397, and p4399 are depicted in FIG. 16A. 3/4 Mimotope vaccines indicated mount detectable immune responses against the original epitope p4373. A similar phenomenon can be detected analysing cross reactivity against the non-modified form as displayed by p4371. The original epitope p4373-vaccine and 2/4 Mimotope vaccines mount relevant titers against p4371. Surprisingly, the mimotopes selected by MV-003, which is specifically binding to p4373 are also inducing a immune reaction cross reacting with the unmodified form of the original epitope.

As example for MV-004-mimotopes, original epitope p4372 and the mimotopes p4417, p4418, p4419, and p4420 are depicted in FIG. 16B. 3/4 Mimotope vaccines shown mount detectable immune responses against the original epitope p4372.

As example for MV-001-mimotopes, original epitope p4371 and the mimotopes p4381, p4382, and p4390 are depicted in FIG. 16C. All Mimotope vaccines depicted mount detectable immune responses against the original epitope p4371. A similar phenomenon as described for MV-003 derived mimotopes can be detected analysing cross reactivity against the pyroglutamate-modified form as displayed by p4373. The original epitope p4371-vaccine and all Mimotope vaccines mount relevant titers against p4373. Surprisingly, the mimotopes selected by MV-001, which is specifically binding to p4371 are inducing a immune reaction cross reacting better with the modified form of the original epitope than the original epitope induced immune reaction or the parental antibody. Thus these mimotopes might surprisingly be able to induce but are not necessarily inducing a broader immune reaction than the parental antibody and can be used for a more wide targeting of forms of Aβ.

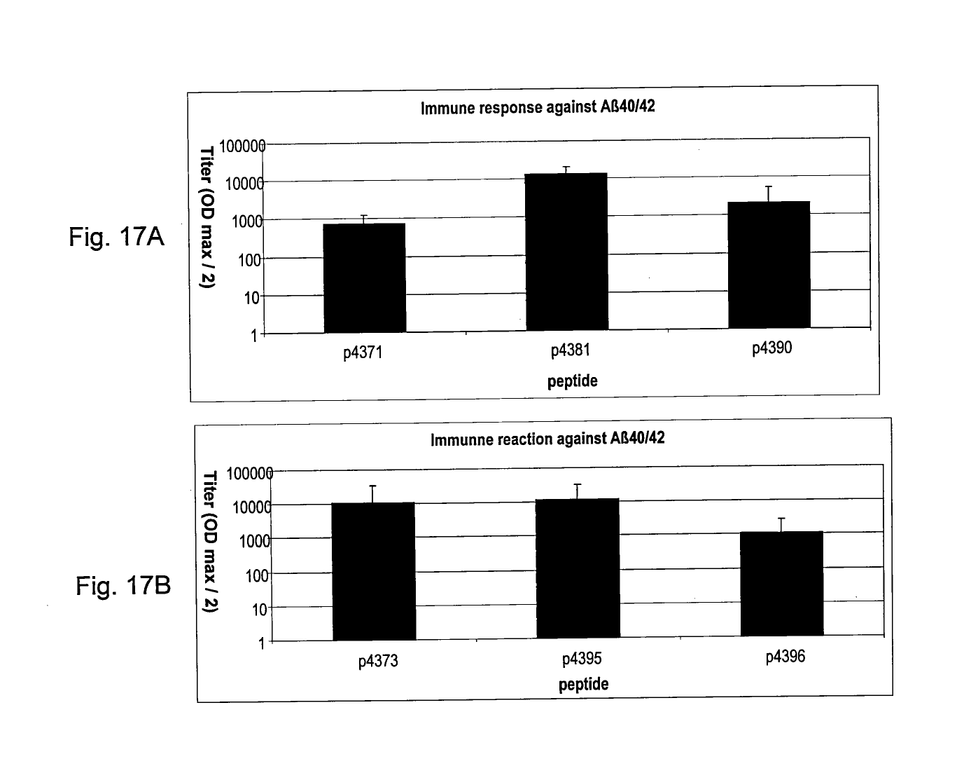

FIG. 17 shows examples for in vivo characterisations of the immune response elicited by mimotope vaccination against full length Aβ. Surprisingly, the mimotopes selected by using MV-001 and MV-003 induce a cross reaction not only with the truncated or modified short epitopes used to create the antibodies but also induce cross reactivity to full length, non modified forms of Aβ as good as the original sequence or even more efficiently than p4371/p4373. For MV-002 original epitope as well as for the mimotopes identified, no such cross reactivity can be detected demonstrating a transfer of specificity of the antibody to the free N-Terminus of unmodified A11-40/42. Thus the mimotopes presented in this invention constitute optimised vaccine candidates to target a broad spectrum of naturally occurring forms of the Aβ peptides as have been found in the brain of AD patients. The forms include but are not limited to Aβ1-40/42, and N-terminally truncated forms like Aβ-40/42, Aβ(pE)_{3-40/42} and unmodified Aβ11-40/42 respectively.

In Table 4 and 5 further examples of the immune response elicited by mimotope vaccination against full length Aβ by using MV-001 and MV-003 derived mimotopes are described.

TABLE 4
In vivo characterisation of mimotopes: MV-001

| Internal Peptide | Detection of Aβ/truncated/modified |
|---|---|
| number | forms |
| p4381 | + |
| p4383 | + |
| p4385 | + |
| p4386 | + |
| p4390 | + |
| p4707 | + |
| p4714 | + |
| p4715 | + |
| p4725 | + |
| p4730 | + |
| p4738 | + |
| p4740 | + |
| p4748 | + |
| p4753 | + |

All peptides listed in Table 4 mount specific immune reactions against full length and/or truncated and modified forms of Aβ or fragments thereof.

TABLE 5
In vivo characterisation of mimotopes: MV-003

| Internal Peptide | Detection of Aβ/truncated/modified |
|---|---|
| number | forms |
| p4395 | + |
| p4396 | + |
| p4397 | + |
| p4399 | + |

All peptides listed in Table 5 mount specific immune reactions against full length and/or truncated and modified forms of Aβ or fragments thereof.

3. Results for MV-002

3.1. Identification of Specific Monoclonal Antibodies (mAB) Directed Against n-Terminally Truncated and Modified Forms of Aβ:

FIG. 21 depicts the characterisation of the monoclonal anti-body MV-002 (internal name A115; IgG2b) derived from experiment Alz-9 demonstrating specificity for full length Aβ and Aβ fragments truncated at position E11 and H14 and modified at position E11 to pE11.

3.2. Screening with Specific mABs Directed Against n-Terminally Truncated and Modified Forms of Aβ:

3.2.1. Phage Display Library Ph.D. 7

3.2.1.1. Screening with Monoclonal Antibody Directed Against p1323

47 Sequences were identified by screening PhD 7 phage display libraries in this screen: Table 1 summarises the peptides identified and their binding capacity as compared to the original epitope.

TABLE 1
mimotopes binding to the parental antibody MV-002

| Internal Peptide | | |
|---|---|---|
| number | Sequence | Binding Capacity |
| p4403 | SHTRLYFC | 1 |
| p4404 | SGEYVFHC | 1 |
| p4413 | SGQLKFPC | 1 |
| p4414 | SGQIWFRC | 1 |
| p4415 | SGEIHFNC | 1 |
| p4666 | GQIWFISC | 1 |
| p4667 | NDAKIVFC | 3 |
| p4668 | GQIIFQSC | 2 |
| p4669 | GQIRFDHC | 3 |
| p4670 | HMRLFFNC | 3 |
| p4671 | GEMWFALC | 3 |
| p4672 | GELQFPPC | 3 |
| p4673 | GELWFPC | 3 |
| p4674 | SHQRLWFC | 3 |
| p4675 | HQKMIFAC | 3 |
| p4676 | GEMQFFIC | 3 |
| p4677 | GELYFRAC | 3 |
| p4678 | GEIRFALC | 3 |
| p4679 | GMIVFPHC | 3 |
| p4680 | GEIWFEGC | 3 |
| p4681 | GEIYFERC | 3 |
| p4682 | AIPLFVMC | 1 |
| p4683 | GDLKFPLC | 3 |
| p4684 | GQILFPVC | 3 |
| p4685 | GELFFPKC | 3 |
| p4686 | GQIMFPRC | 3 |
| p4687 | HMRMYFEC | 3 |
| p4688 | GSLFFWPC | 2 |
| p4689 | GEILFGMC | 3 |
| p4690 | GQLKFPFC | 3 |
| p4691 | KLPLFVMC | 1 |
| p4692 | GTIFFRDC | 1 |
| p4693 | THQRLWFC | 3 |
| p4694 | GQIKFAQC | 3 |
| p4695 | GTLIFHHC | 2 |
| p4696 | GEIRFGSC | 3 |
| p4697 | GQIQFPLC | 3 |
| p4698 | GEIKFDHC | 3 |
| p4699 | GEIQFGAC | 3 |
| p4700 | QLPLFVLC | 1 |
| p4794 | HQKMIFC | 2 |
| p4795 | GELFFEKC | 2 |
| p4796 | GEIRFELC | 2 |
| p4804 | Ac-GEIYFERC | 2 |
| p4805 | SGEIYFERC | 1 |
| p4806 | AGEIYFERC | 1 |
| p4807 | CGEIYFER | 1 |

Legend to Table 1: the binding capacity is coded by the following binding code: 1:X describes the dilution factor of the parental AB. Ac- . . . indicates acetylated AA.

| | | OD halfmax |
|---|---|---|
| binding code | | 1:X |
| 0 | no binding | :0 |
| 1 | weak binding | :<40000 |
| 2 | medium binding | :40000-320000 |
| 3 | strong binding | :>320000 |

3.3. In Vitro Characterisation of Mimotopes Identified in Screening Phage Display Libraries with Monoclonal Antibodies Directed Against n-Terminally Truncated and Modified Forms of Aβ:

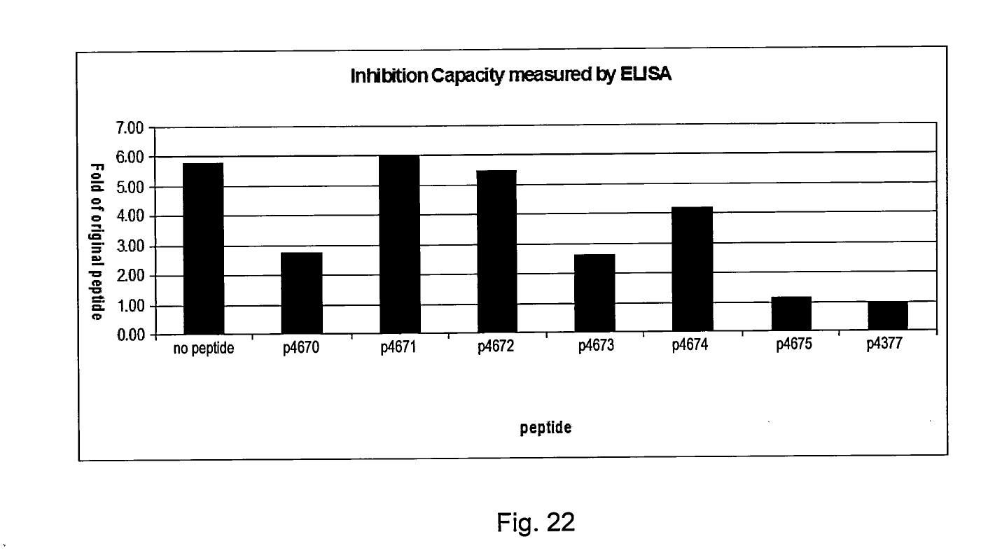

FIGS. 21 and 22 show representative examples for binding and inhibition assays used to characterise mimotopes in vitro. Data obtained are summarised in Tables 1 and 2 respectively.

MV-002 Mimotopes: From the 47 sequences presented 11 sequences inhibited binding of the monoclonal antibody MV-002 in in vitro competition experiments. Additional 36 sequences were identified that did not inhibit binding of monoclonal antibody in in vitro competition experiments but still retained binding capacity to the parental antibody (Table 2). Importantly, as described in FIGS. 23-25, the ability to compete with the original epitope for binding to the parental antibody in vitro was no prerequisite to mount specific immune responses cross reacting with specific peptides in vivo. Thus inhibiting as well as non-inhibiting peptides can be used for inducing immune responses detecting peptides in vivo (for details see: FIGS. 23-25) which can lead to clearance of amyloid peptides from the brain.

TABLE 2
mimotopes identified in this
invention giving positive results in
inhibiting assays; MV-002 Mimotopes

| Internal | | |
|---|---|---|
| Peptide | | Inhibition |
| number | Sequence | Capacity |
| p4667 | NDAKIVFC | 1 |
| p4670 | HMRLFFNC | 1 |
| p4673 | GELWFPC | 1 |
| p4674 | SHQRLWFC | 1 |
| p4675 | HQKMIFAC | 2 |
| p4680 | GEIWFEGC | 2 |
| p4681 | GEIYFERC | 2 |
| p4689 | GEILFGMC | 1 |
| p4698 | GEIKFDHC | 2 |
| p4699 | GEIQFGAC | 1 |
| p4794 | HQKMIFC | 1 |

Legend to Table 2: the inhibition capacity is coded by the following code:
Weak inhibition means more peptide is required to lower AB binding than with the original epitope; strong inhibition means similar peptide amounts are required for mimotope and original epitope for lowering AB binding. Mimotopes are compared to the original peptide as standard. OD at 5 ug peptide used in the assay is used to calculate the competition capacity compared to original peptide.

| competition | |
|---|---|
| code | |
| 0 | no inhibition (OD of peptide above 4, 6 times of original |
| | peptide) |
| 1 | Weaker than original epitope (OD of peptide below 4, 6 |
| | times of original peptide) |
| 2 | strong inhibition (as original epitope; OD of peptide below |
| | 2, 3 times of original peptide) |

3.4. In Vivo Characterisation of Mimotopes Identified in Screening Phage Display Libraries with a Monoclonal Antibody Directed Against Amyloid Beta:

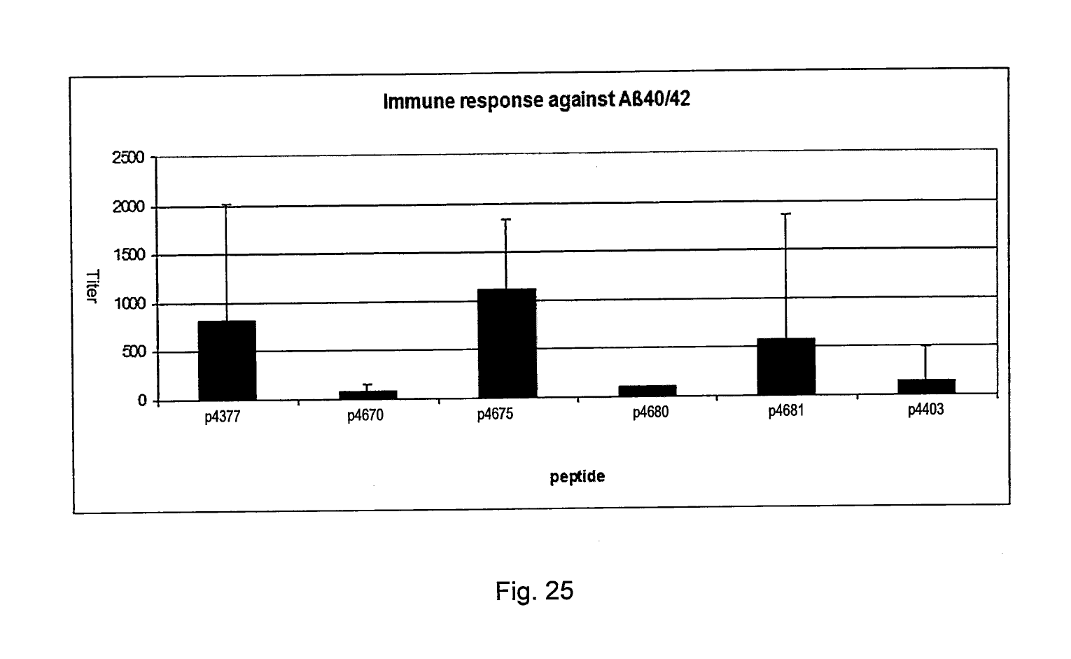

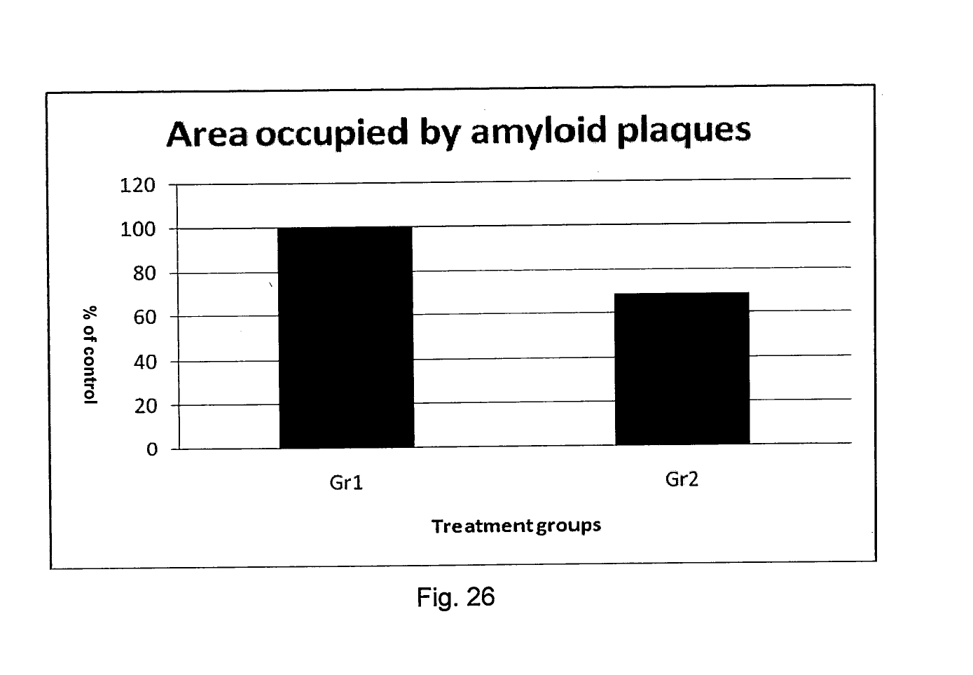

Female C57/b16 mice, 5-6 mice per group, were subcutaneously immunized with 30 μg peptide coupled to KLH. Control groups were administered original epitope-KLH conjugates respectively. As adjuvant alum was used (always 1 mg per mouse). The peptides administered were all able to bind to monoclonal antibodies specifically although some of the peptides did not inhibit the binding of the original epitope to its parental antibody in vitro (in an in vitro inhibition assay). The in vitro ELISA assay to determine the antibody titer was performed with sera of single mice after each vaccination in a two week interval (see FIGS. 25 and 26 respectively). Titers were calculated as OD max/2 in all figures shown. The wells of the ELISA plate were coated with mimotope-BSA conjugate and an irrelevant peptide-BSA conjugate (negative control). The positive control was performed by reaction of the parental antibody with the respective mimotope-BSA conjugate. The detection was performed with anti-mouse IgG. Additionally, recombinant proteins were immobilised on ELISA plates and sera reacted accordingly. FIGS. 23, 24 and 25 show representative examples for assays used to characterise mimotopes in vivo. The results depicted were derived from peptides active in in vitro inhibition assays like p4670, p4675, p4680, and p4681 and a peptide without inhibition capacity, p4403 respectively.

FIG. 23 shows examples for in vivo characterisations of the immune response elicited by mimotope vaccination by analysing the immune response against injected peptide and an irrelevant peptide, containing an unrelated sequence. In the examples shown, the epitope p4377 and the mimotopes p4670, p4675, p4680, p4681 and p4403 elicited immune responses against the injected peptides but failed to induce a relevant unspecific immune response against an unrelated sequence (p1454).

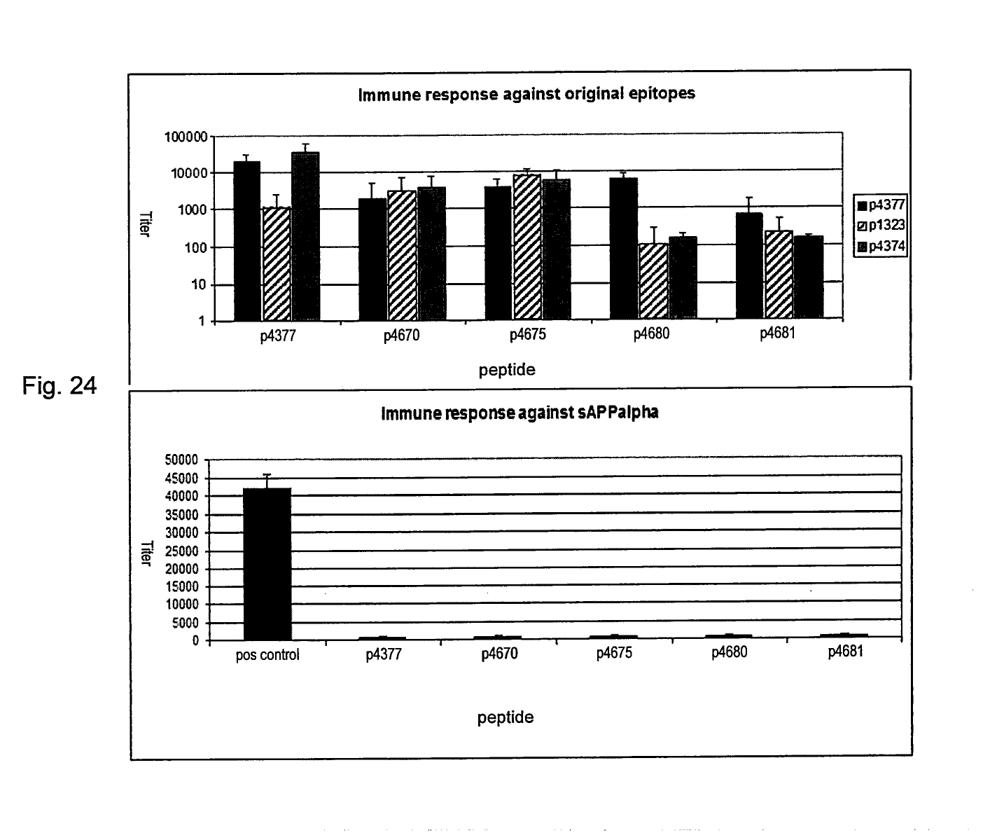

FIG. 24 shows examples for in vivo characterisations of the immune response elicited by mimotope vaccination against the respective original epitope of the parental antibody (p4377) as well as against peptides derived from truncated species of Aβ (p1323 and p4374) and against sAPP alpha. p4377 and the mimotopes p4670, p4675, p4680, p4681 and p4403 mounted detectable immune responses against the original epitope

p4377. A similar phenomenon could be detected analysing cross reactivity against the modified form as displayed by p4374. Interestingly, the original epitope and the mimotope vaccines mounted relevant titers against p4374 the modified form of the original epitope. Surprisingly, the mimotopes seemed to be able to induce but did not necessarily induce a more efficient immune response against p1323 indicating a potential to induce a broader immuno-reactivity as compared to the original Aβ fragment. Additionally, no reactivity was detectable against sAPP alpha.

FIG. 25 shows examples for in vivo characterisations of the immune response elicited by mimotope vaccination against full length Aβ. Surprisingly, the mimotopes selected by using MV-002 induced a cross reaction not only with the truncated or modified short epitopes used to create the antibodies but also induced cross reactivity to full length, non modified forms of Aβ as good as the original sequence or even more efficiently than p4377.

Interestingly competing as well as non competing peptides were able to induce similar immune responses specifically interacting with peptides containing original Aβ sequences. Thus the mimotopes presented in this invention constitute optimised, novel vaccine candidates to target a broad spectrum of naturally occurring forms of the Aβ peptides as have been found in the brain of AD patients. The forms include but are not limited to Aβ1-40/42, and N-terminally truncated forms like Aβ3-40/42, Aβ(pE)_{3-40/42}, unmodified Aβ11-40/42, modified Aβp(E)11-40/42 and Aβ14-40/42 respectively. Importantly, the mimotopes presented also did not induce a cross reactivity to the neoepitopes present in sAPP alpha after cleavage from APP and thus do not interfere with normal sAPP alpha signalling (see FIG. 24 for details)

TABLE 3
Non-Mimotope peptides used

| Internal | |
|---|---|
| Peptide no. | Sequence |
| p1253 | DAEFRHDSGYC |
| p1323 | CHQKLVFFAED |
| p4374 | p(E)VHHQKLVFC |
| p4377 | EVHHQKLVFC |
| p1454 | CGLMVGGVV |
| Aβ1-40 | DAEFRHDSGYEVHHQKLVFFAEDVGSNKGAIIGL |
| | MVGGVV; |
| | derived from human APP (gi:112927) |
| Aβ1-42 | DAEFRHDSGYEVHHQKLVFFAEDVGSNKGAIIGL |
| | MVGGVVIA; |
| | derived from human APP (gi:112927) |
| sAPPalpha | alpha-Secretase induced cleavage |
| | product derived from human APP |
| | (gi:112927) |

In Table 4 further examples of the immune response elicited by mimotope vaccination against full length Aβ by using MV-002 derived mimotopes are described. All peptides listed in table 4 mount specific immune reactions against full length and/or truncated and modified forms of Aβ or fragments thereof.

TABLE 4
In vivo characterisation of mimotopes: MV-002

| Internal Peptide | Detection of Aβ/truncated/modified |
|---|---|
| number | forms |
| p4403 | + |
| p4404 | + |
| p4413 | + |
| p4414 | + |
| p4415 | + |
| p4670 | + |
| p4673 | + |
| p4675 | + |
| p4680 | + |
| p4681 | + |
| p4693 | + |
| p4696 | + |
| p4698 | + |
| p4699 | + |

### Example 9

### In Vivo Characterisation of Mimotopes for the Efficacy to Reduce AD Like Disease in Transgenic Animals

The Tg2576 AD mouse model was used to study the preclinical efficacy of the mimotope vaccines. This transgenic line is expressing human APP carrying the Swedish double mutation at aa position 670/671 under the control of a hamster prion protein (PrP) promoter which results in overexpression of the protein. It is currently one of the most widely employed models in AD research. The Tg2576 model recapitulates various hallmarks of AD pathology including disease-specific amyloid plaque deposition and astrocytosis. As all other AD model systems available to date, it does not reflect all cardinal neuropathological features of AD.

To assess whether treatment with mimotopes is capable of preventing cerebral Aβ accumulation, Tg2576 mice were s.c. injected 6 times at monthly intervals with peptide-KLH conjugates adsorbed to ALUM (adjuvant: aluminium hydroxide) or PBS adsorbed to ALUM (referred to as PBS or control) alone. Up to eight weeks after the last immunization, animals were sacrificed, their brains harvested and analyzed for their Aβ load (AD-like pathology). The mice were sacrificed under deep anaesthesia. Subsequently, the brain was isolated, fixed in 4% PFA and dehydrated by graded Ethanol series followed by incubation in Xylene and paraffin embedding. Each paraffin-embedded brain was sectioned at 7 μM using a slicing microtome and sections were mounted on glass slides.

As a method to assay AD-like pathology in Tg2576 animals, the relative area occupied by amyloid deposits in the brain of treated animals was analyzed. This analysis was performed using an automated area recognition programme. To identify the plaques, sections were stained with the monoclonal antibody (mAb) 3A5 (specific for A940/42). Mimotope treated animals were compared to control animals. All animals have been sacrificed at an age of 13, 5-14 months. For this analysis 3 slides/animal covering the cortex and hippocampus were selected, stained with mAb 3A5 and subsequently documented using the Mirax-system (Zeiss). For the calculation of the area occupied by amyloid plaques, up to four individual sections per slide were analysed and sections carrying tissue artefacts and aberrant staining intensities have been excluded after inspection of the result pictures.

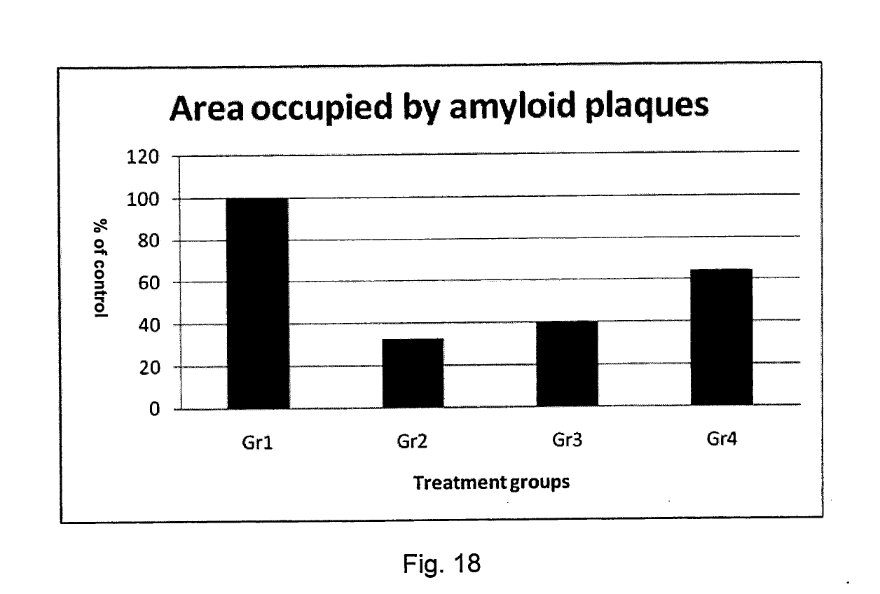

For the mimotopes derived from MV001 an area analysis using three exemplary candidates was performed: Analysis was performed following repeated vaccination using peptide-KLH conjugate vaccines. The control group showed an average occupation of 0.35% as compared to 0.11%, 0.14% and 0.22% for the mimotope treated animals respectively. This corresponds to a reduction following mimotope treatment of 67% in group 2, a 60% reduction in group 3 and a 36% reduction in group 4 (see FIG. 18).

For the mimotopes of MV002 an area analysis using one exemplary candidate was performed: Analysis was performed following repeated vaccination using peptide-KLH conjugate vaccines. The control group showed an average occupation of 0.35% as compared to 0.24% for the mimotope treated animals respectively. This corresponds to a reduction following mimotope treatment of 31% in group 2.

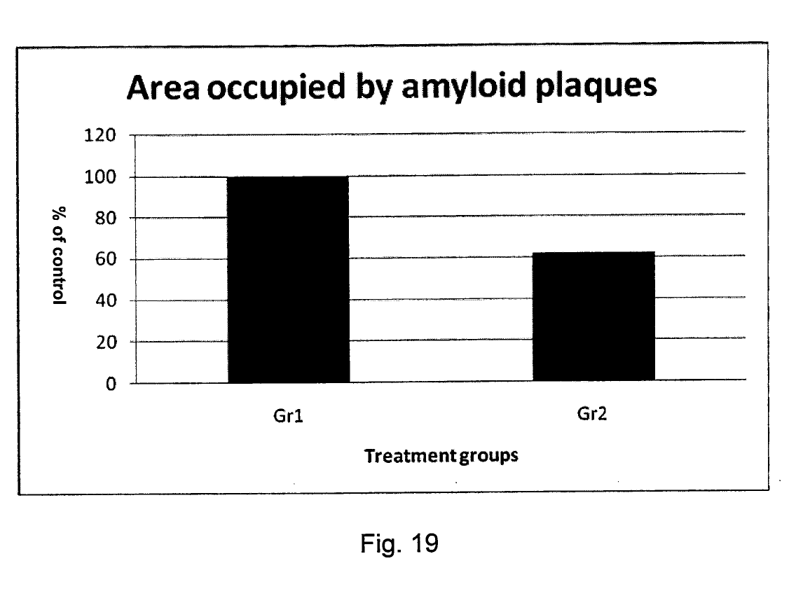

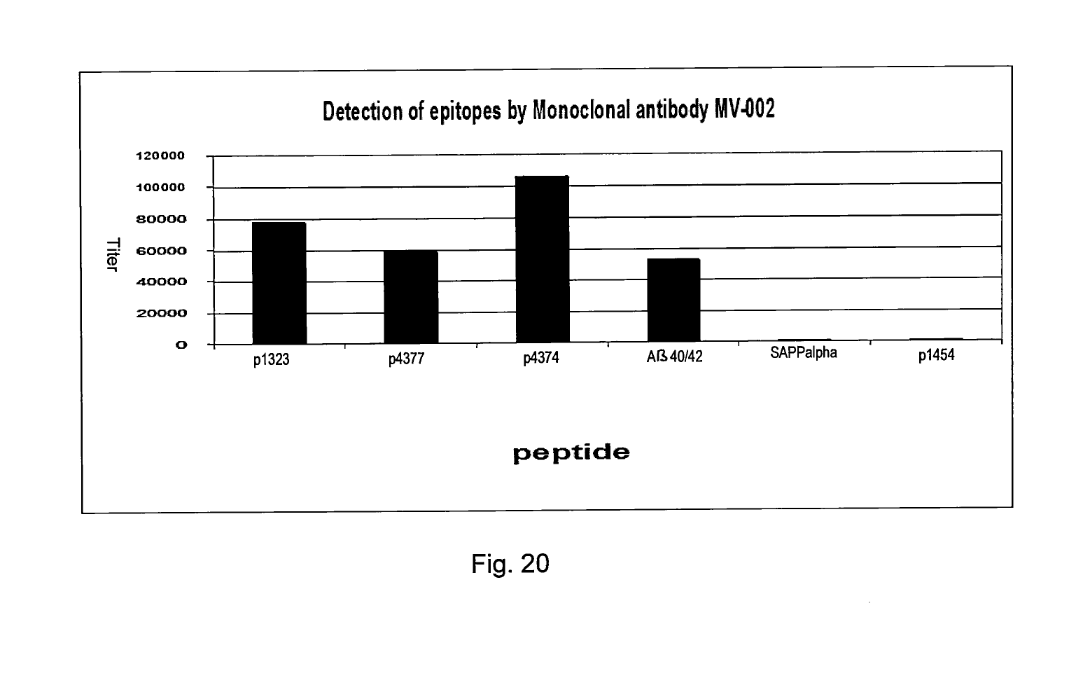

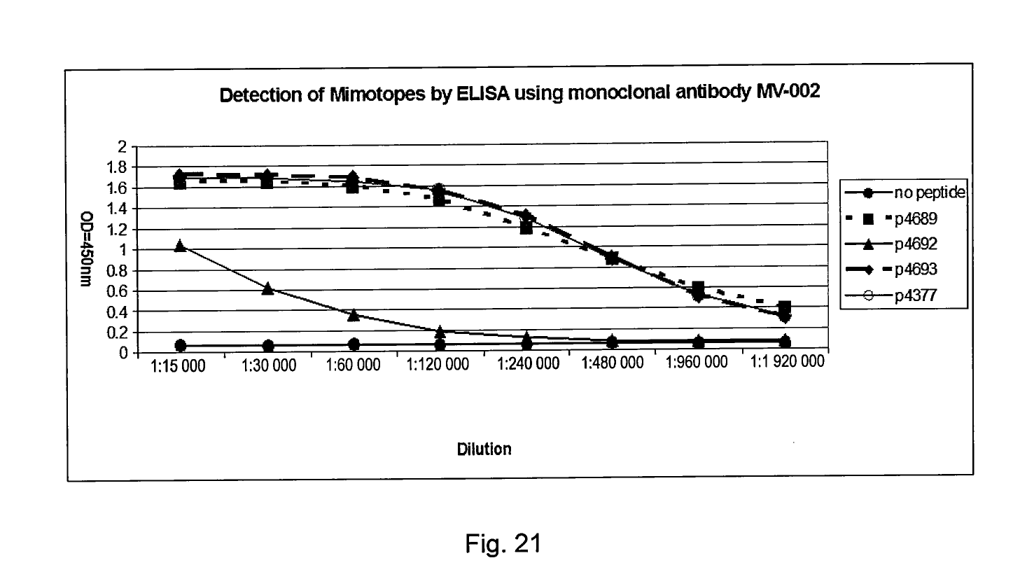

A similar picture can be detected for the group of MV003 derived mimotopes. Here the example of p4395 is depicted. As described for the MV001 derived mimotopes, an analysis of the area occupied by amyloid plaques following peptide-conjugate vaccination has been performed. The control group showed an average α-cupation of 0.35% as compared to 0.21% for the mimotope treated animals respectively. This corresponds to a reduction following mimotope treatment of 38% in group 2 (see FIG. 19).

Thus, this set of data clearly indicates a beneficial effect of mimotope vaccine treatment on AD like pathology in transgenic animals.

### Example 10

### In Vivo Characterisation of Mimotopes for the Efficacy to Reduce PD Like Disease in Transgenic Animals (Proof of Concept Analysis)

The double transgenic mouse model (mThy1-APP751 (line TASD41) crossed with mThy1-wt human a-syn (Line TASD 61)) was used to study the preclinical efficacy of AD mimotope vaccines to reduce PD like disease. The model recapitulates various hallmarks of AD and PD pathology including disease-specific amyloid plaque deposition and astrocytosis as well as synuclein aggregation and cell loss.

To assess whether treatment with mimotopes is capable of ameliorating PD like disease, transgenic mice were s.c. injected 6 times at monthly intervals with peptide-KLH conjugates adsorbed to ALUM (adjuvant: aluminium hydroxide) or PBS adsorbed to ALUM (referred to as PBS or control) alone. After the last immunization, animals were sacrificed following guidelines for the humane treatment of animals. Subsequently, the brain was isolated, fixed and sectioned at 40 μM using a vibratome and sections were stored at −20° C. in cryoprotective medium. Sections were immunostained with antibodies against α-synuclein and NeuN (neuronal marker) and imaged with the laser confocal microscope. Digital images were analyzed with the ImageQuant program to assess numbers of α-synuclein aggregates and neurons. Mimotope treated animals were compared to control animals. Results depict an exemplary set of data for a mimotope described in this invention

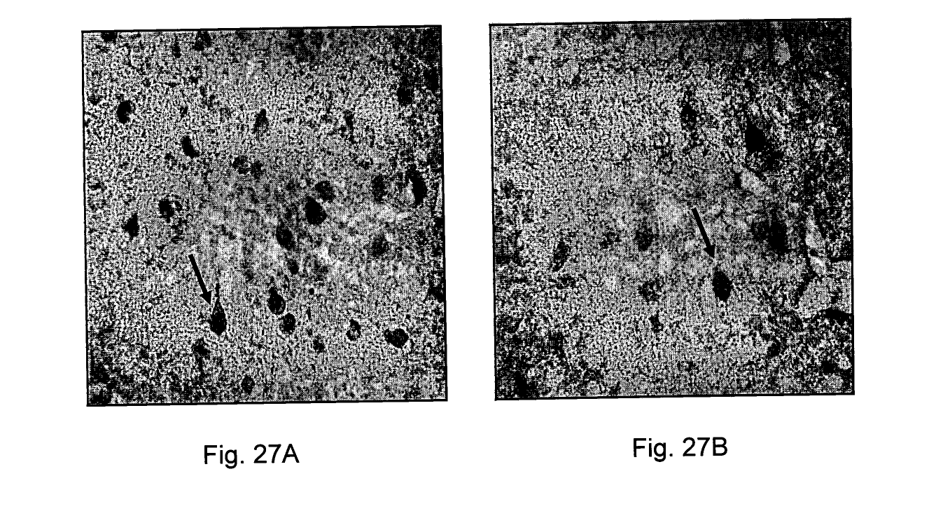

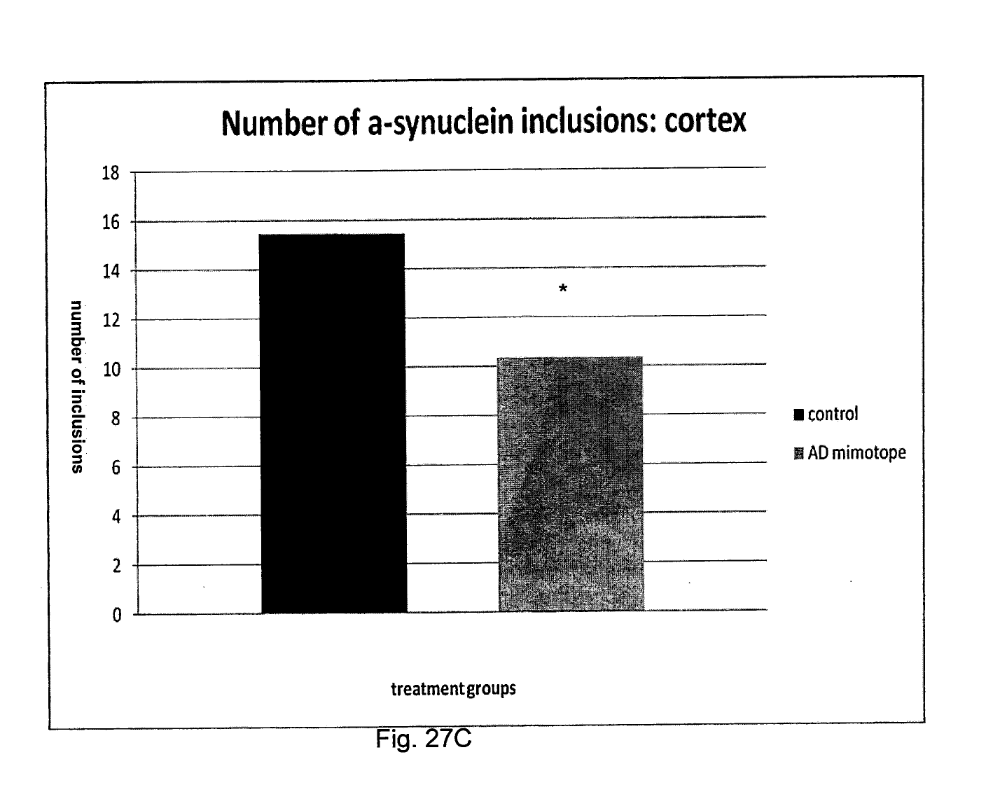

In order to analyse whether vaccination with AD mimotopes would result in a reduction of PD associated pathology the incidence of neuronal inclusions of α-synuclein in the frontal cortex and the hippocampus was analysed (Lewy body like inclusions). Animals overexpressing APP and α-synuclein in the brain developed pathologic alterations reminiscent of PD. α-synuclein positive neuronal inclusions are depicted in FIG. 27 as spots in neuronal bodies. A quantitative analysis of the inclusions revealed that the levels of accumulation of α-synuclein in the neuronal cell bodies in the neocortex and hippocampus were significantly reduced in the double transgenic mice following AD mimotope vaccination. This reduction amounted to 32.7% in the cortex (p=0.0001) indicating a beneficial effect of AD mimotope vaccination on PD like pathology in this area.

As a second method to assay PD-like pathology in transgenic animals, the number of neurons in the cortex and hippocampus of treated animals by NeuN staining was analyzed.

In this animal model a progressive loss of neurons in the frontal cortex as well as in the hippocampus upon ageing can be detected. Quantification of the neuronal density in the frontal cortex and the hippocampus showed a slight decrease in double transgenic PBS treated mice as compared to non transgenic control animals. This slight reduction indicates neurodegeneration in the strain used for this experiment.

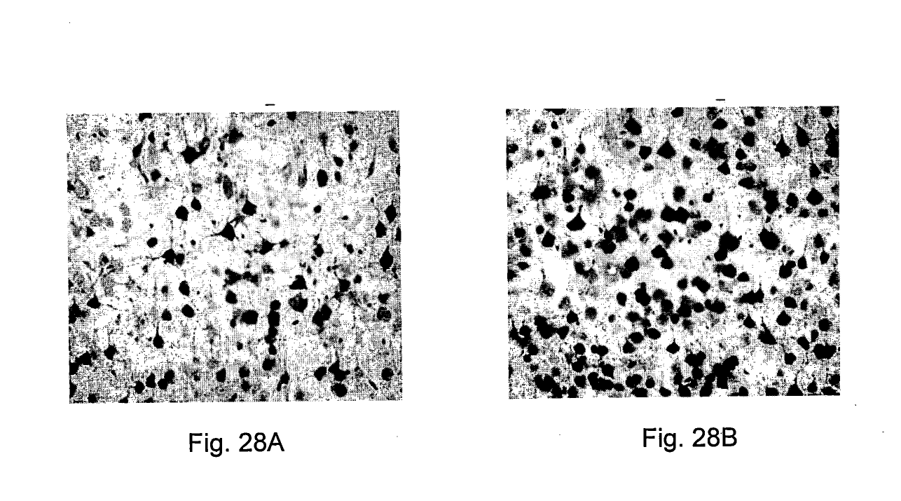

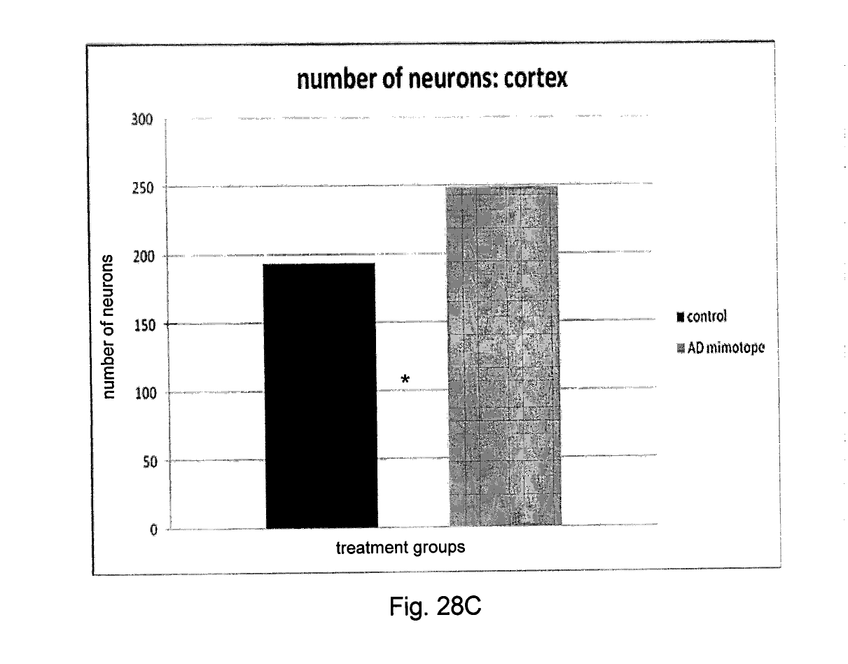

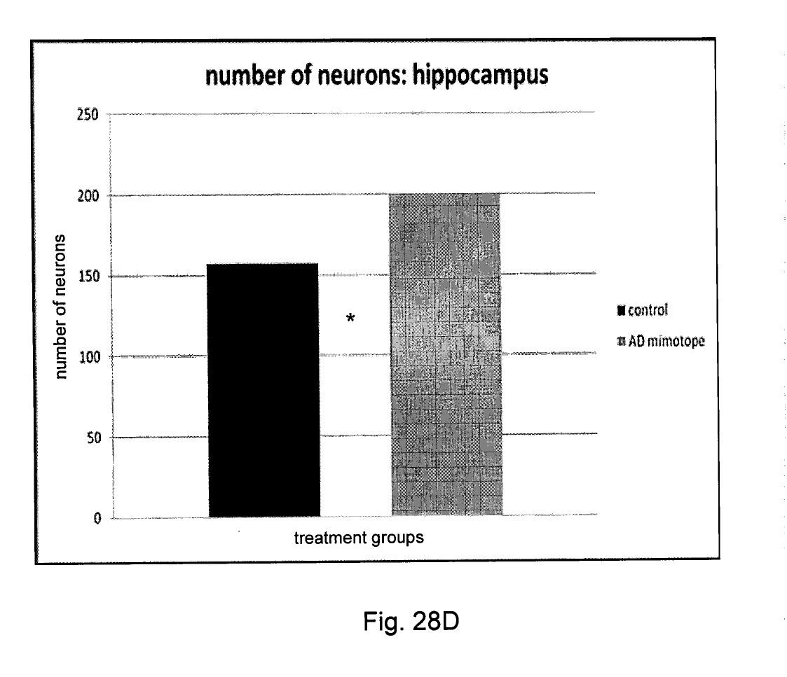

Interestingly, mice treated with an AD mimotope (FIG. 28) showed levels of NeuN positive neurons, which were comparable to controls. Double Tg animals revealed a statistically significant 27% increase (p=0.044) in the hippocampus as compared to the carrier treated controls respectively. In the cortical area, a 28.4% (p=0.0053) increase in the double Tg animals could be observed following AD mimotope treatment. This relative increase as compared to the vehicle treated animals could also be interpreted as an indication of reduced neurodegeneration in successfully treated animals.

Summarizing, this set of data clearly indicates a beneficial effect of AD mimotope vaccine treatment on PD like symptoms in transgenic animals.

1-17. (canceled)

18. A method for treating, preventing and/or ameliorating at least one motor symptom of Parkinson's disease in a subject in need thereof, wherein said method comprises administering to said subject an effective amount of a peptide having a binding capacity to an antibody which is specific for an epitope of an amyloid-beta-peptide (A13).

19. The method of claim 18, wherein the epitope of the amyloid-beta-peptide has a sequence of DAEFRH (SEQ ID NO. 26), EFRHDSGY (SEQ ID NO. 156), pEFRHDSGY (SEQ ID NO. 146), EVHHQKL (SEQ ID NO. 158), HQKLVF (SEQ ID NO: 215) or HQKLVFFAED (SEQ ID NO. 216).

20. The method of claim 18, wherein the peptide does not comprise an amino acid sequence of DAEFRH (SEQ ID NO. 26), EFRHDSGY (SEQ ID NO. 156), pEFRHDSGY (SEQ ID NO. 146), EVHHQKL (SEQ ID NO. 158), HQKLVF (SEQ ID NO: 215) or HQKLVFFAED (SEQ ID NO. 216).

21. The method of claim 18, wherein the peptide comprises an amino acid sequence of

| (SEQ ID NO: 217) | |
|---|---|
| X₁X₂X₃X₄X₅X₆X₇, | (Formula I) |

wherein
X₁ is G or an amino acid with a hydroxy group or a negatively charged amino acid,
X₂ is a hydrophobic amino acid or a positively charged amino acid,
X₃ is a negatively charged amino acid,
X₄ is an aromatic amino acid or a hydrophobic amino acid or leucine (L),
X₅ is histidine (H), lysine (K), tyrosine (Y), phenylalanine (F) or arginine (R), and
X₆ is not present or serine (S), threonine (T), asparagine (N), glutamine (Q), aspartic acid (D), glutamic acid (E), arginine (R), isoleucine (I), lysine (K), tyrosine (Y), or glycine (G), and
X₇ is not present or any amino acid.

22. The method of claim 18, wherein the peptide comprises an amino acid sequence of

| (SEQ ID NO: 154) | |
|---|---|
| X₁RX₂DX₃(X₄)ₙ(X₅)ₘ(X6)ₒ, | (Formula II), |

wherein
X1 is isoleucine (I) or valine (V),
X₂ is tryptophan (W) or tyrosine (Y),
X₃ is threonine (T), valine (V), alanine (A), methionine (M), glutamine (Q) or glycine (G),
X₄ is proline (P), alanine (A), tyrosine (Y), serine (S), cysteine (C) or glycine (G),
X₅ is proline (P), leucine (L), glycine (G) or cysteine (C),
X₆ is cysteine (C), and
n, m and o are, independently, 0 or 1.

23. The method of claim 18, wherein the peptide comprises an amino acid sequence of

| (SEQ ID NO: 155) | |
|---|---|
| EX₁WHX₂X₃(X₄)ₙ(X₅)ₘ | (Formula III), |

wherein
X₁ is valine (V), arginine (R) or leucine (L),
X₂ is arginine (R) or glutamic acid (E),
X₃ is alanine (A), histidine (H), lysine (K), leucine (L), tyrosine (Y) or glycine (G),
X₄ is proline (P), histidine (H), phenylalanine (F) or glutamine (Q) or Cysteine
X₅ is cysteine (C), and
n and m are, independently, 0 or 1.

24. The method of claim 18, wherein the peptide comprises an amino acid sequence of QDFRHY(C) (SEQ ID NO. 236), SEFKHG(C) (SEQ ID NO. 237), TSFRHG(C) (SEQ ID NO. 238), TSVFRH(C) (SEQ ID NO. 239), TPFRHT(C) (SEQ ID NO. 240), SQFRHY(C) (SEQ ID NO. 241), LMFRHN(C) (SEQ ID NO. 242), SAFRHH(C) (SEQ ID NO. 243), LPFRHG(C) (SEQ ID NO. 244), SHFRHG(C) (SEQ ID NO. 245), ILFRHG(C) (SEQ ID NO. 246), QFKHDL(C) (SEQ ID NO. 247), NWFPHP(C) (SEQ ID NO. 248), EEFKYS(C) (SEQ ID NO. 249), NELRHST(C) (SEQ ID NO. 250), GEMRHQP(C) (SEQ ID NO. 251), DTYFPRS(C) (SEQ ID NO. 252), VELRHSR(C) (SEQ ID NO. 253), YSMRHDA(C) (SEQ ID NO. 254), AANYFPR(C) (SEQ ID NO. 255), SPNQFRH(C) (SEQ ID NO. 256), SSSFFPR(C) (SEQ ID NO. 257), EDWFFWH(C) (SEQ ID NO. 258), SAGSFRH(C) (SEQ ID NO. 259), QVMRHHA(C) (SEQ ID NO. 260), SEFSHSS(C) (SEQ ID NO. 261), QPNLFYH(C) (SEQ ID NO. 262), ELFKHHL(C) (SEQ ID NO. 263), TLHEFRH(C) (SEQ ID NO. 264), ATFRHSP(C) (SEQ ID NO. 265), APMYFPH(C) (SEQ ID NO. 266), TYFSHSL(C) (SEQ ID NO. 267), HEPLFSH(C) (SEQ ID NO. 268), SLMRHSS(C) (SEQ ID NO. 269), EFLRHTL(C) (SEQ ID NO. 270), ATPLFRH(C) (SEQ ID NO. 271), QELKRYY(C) (SEQ ID NO. 272), THTDFRH(C) (SEQ ID NO. 273), LHIPFRH(C) (SEQ ID NO. 274), NELFKHF(C) (SEQ ID NO. 275), SQYFPRP(C) (SEQ ID NO. 276), DEHPFRH(C) (SEQ ID NO. 277), MLPFRHG(C) (SEQ ID NO. 278), SAMRHSL(C) (SEQ ID NO. 279), TPLMFWH(C) (SEQ ID NO. 280), LQFKHST(C) (SEQ ID NO. 281), ATFRHST(C) (SEQ ID NO. 282), TGLMFKH(C) (SEQ ID NO. 283), AEFSHWH(C) (SEQ ID NO. 284), QSEFKHW(C) (SEQ ID NO. 285), AEFMHSV(C) (SEQ ID NO. 286), ADHDFRH(C) (SEQ ID NO. 287), DGLLFKH(C) (SEQ ID NO. 288), IGFRHDS(C) (SEQ ID NO. 289), SNSEFRR(C) (SEQ ID NO. 290), SELRHST(C) (SEQ ID NO. 291), THMEFRR(C) (SEQ ID NO. 292), EELRHSV(C) (SEQ ID NO. 293), QLFKHSP(C) (SEQ ID NO. 294), YEFRHAQ(C) (SEQ ID NO. 295), SNFRHSV(C) (SEQ ID NO. 296), APIQFRH(C) (SEQ ID NO. 297), AYFPHTS(C) (SEQ ID NO. 298), NSSELRH(C) (SEQ ID NO. 299), TEFRHKA(C) (SEQ ID NO. 300), TSTEMWH(C) (SEQ ID NO. 301), SQSYFKH(C) (SEQ ID NO. 302), (C)SEFKH (SEQ ID NO. 303), SEFKH(C) (SEQ ID NO. 304), (C)HEFRH (SEQ ID NO. 305) or HEFRH(C) (SEQ ID NO. 306).

25. The method of claim 18, wherein the peptide comprises an amino acid sequence of

| (SEQ ID NO: 213) | |
|---|---|
| (X₁)ₘGX₂X₃X₄FX₅X₆(X₇)ₙ | (Formula IV), |

wherein
X₁ is serine (S), alanine (A) or cysteine (c),
X₂ is serine (S), threonine (T), glutamic acid (E), aspartic acid (D), glutamine (Q) or methionine (M),
X₃ is isoleucine (I), tyrosine (Y), methionine (M) or leucine (L),
X₄ is leucine (L), arginine (R), glutamine (Q), tryptophan (W), valine (V), histidine (H), tyrosine (Y), isoleucine (I), lysine (K) methionine (M) or phenylalanine (F),
X₅ is alanine (A), phenylalanine (F), histidine (H), asparagine (N), arginine (R), glutamic acid (E), isoleucine (I), glutamine (Q), aspartic acid (D), proline (P) tryptophan (W), or glycine (G)
X₆ is any amino acid residue,
X₇ is cysteine (C), and
m and n are, independently, 0 or 1.

26. The method of claim 18, wherein the peptide comprises an amino acid sequence of

| (SEQ ID NO: 342) | |
|---|---|
| (X₁)ₘHX₂X₃X₄X₅FX₆(X₇)ₙ | (Formula V), |

wherein
X₁ is serine (S), threonine (T) or cysteine (C),
X₂ is glutamine (Q), threonine (T) or methionine (M),
X₃ is lysine (K) or arginine (R),
X₄ is leucine (L), methionine (M),
X₅ is tryptophan (W), tyrosine (Y), phenylalanine (F) or isoleucine (I),
X₆ is asparagine (N), glutamic acid (E), alanine (A) or cysteine (C),
X₇ is cysteine (C), and
n and m are, independently, 0 or 1.

27. The method of claim 18, wherein the peptide comprises an amino acid sequence of AIPLFVM(C) (SEQ ID NO. 350), KLPLFVM(C) (SEQ ID NO. 351), QLPLFVL(C) (SEQ ID NO. 352) or NDAKIVF(C) (SEQ ID NO. 353).

28. The method of claim 18, which said peptide comprises 4 to 30 amino acid residues.

29. The method of claim 18, wherein said peptide is coupled to a pharmaceutically acceptable carrier.

30. The method of claim 29, wherein the pharmaceutically acceptable carrier is KLH (Keyhole Limpet Hemocyanin).

31. The method of claim 18, wherein said administering is subcutaneous, intradermal or intramuscular.

32. The method of claim 18, wherein said peptide is administered with an adjuvant.

33. The method of claim 32, wherein the adjuvant is aluminum hydroxide.

34. The method of claim 18, wherein said effective amounts ranges from 0.1 ng to 10 mg.

35. The method of claim 34, wherein said effective amounts ranges from 100 ng to 10 μg.

36. The method of claim 18, wherein the motor symptom of Parkinson's disease is selected from the group consisting of resting tremor, Bradykinesia, rigidity, postural instability, stooped posture, dystonia, fatigue, impaired fine motor dexterity and motor coordination, impaired gross motor coordination, poverty of movement (decreased arm swing), akathisia, speech problems, loss of facial expression, micrographia, difficulty swallowing, sexual dysfunction and drooling.

37. The method of claim 18, wherein the peptide comprises an amino acid sequence of
EIDYHR (SEQ ID NO. 1), ELDYHR (SEQ ID NO. 2), EVDYHR (SEQ ID NO. 3), DIDYHR (SEQ ID NO. 4), DLDYHR (SEQ ID NO. 5), DVDYHR (SEQ ID NO. 6), DIDYRR (SEQ ID NO. 7), DLDYRR (SEQ ID NO. 8), DVDYRR (SEQ ID NO. 9), DKELR1 (SEQ ID NO. 10), DWELR1 (SEQ ID NO. 11), YREFFI (SEQ ID NO. 218), YREFR1 (SEQ ID NO. 12), YAEFRG (SEQ ID NO. 13), EAEFRG (SEQ ID NO. 14), DYEFRG (SEQ ID NO. 15), ELEFRG (SEQ ID NO. 16), DRELRI (SEQ ID NO. 17), DKELKI (SEQ ID NO: 18), DRELKI (SEQ ID NO. 19), GREFRN (SEQ ID NO. 20), EYEFRG (SEQ ID NO. 21), DWEFRDA (SEQ ID NO. 22), SWEFRT (SEQ ID NO. 23), DKELR (SEQ ID NO. 24), SFEFRG (SEQ ID NO. 25), DAEFRWP (SEQ ID NO. 27), DNEFRSP (SEQ ID NO. 28), GSEFRDY (SEQ ID NO. 29), GAEFRFT (SEQ ID NO. 30), SAEFRTQ (SEQ ID NO. 31), SAEFRAT (SEQ ID NO. 32), SWEFRNP (SEQ ID NO. 33), SWEFRLY (SEQ ID NO. 34), SWELRQA (SEQ ID NO. 35), SVEFRYH (SEQ ID NO. 36), SYEFRHH (SEQ ID NO. 37), SQEFRTP (SEQ ID NO. 38), SSEFRVS (SEQ ID NO. 39), DWEFRD (SEQ ID NO. 40), DAELRY (SEQ ID NO. 41), DWELRQ (SEQ ID NO. 42), SLEFRF (SEQ ID NO. 43), GPEFRW (SEQ ID NO. 44), GKEFRT (SEQ ID NO. 45), AYEFRH (SEQ ID NO. 46), DKE(Nle)R (SEQ ID NO. 47), DKE(Nva)R (SEQ ID NO. 48), DKE(Cha)R (SEQ ID NO: 49),
IRWDTP(C) (SEQ ID NO. 219), VRWDVYP(C) (SEQ ID NO. 220), IRYDAPL(C) (SEQ ID NO. 221), IRYDMAG(C) (SEQ ID NO. 222), IRWDTSL(C) (SEQ ID NO. 223), IRWDQP(C) (SEQ ID NO. 224), IRWDG(C) (SEQ ID NO. 225), IRWDGG(C) (SEQ ID NO. 226),
EVWHRHQ(C) (SEQ ID NO. 227), ERHEKH(C) (SEQ ID NO. 228), EVWHRLQ(C) (SEQ ID NO. 229), ELWHRYP(C) (SEQ ID NO. 230), ELWHRAF(C) (SEQ ID NO. 231), ELWHRA(C) (SEQ ID NO. 232), EVWHRG(C) (SEQ ID NO. 233), EVWHRH(C) (SEQ ID NO. 234) and ERHEK(C) (SEQ ID NO. 235), preferably EVWHRHQ(C) (SEQ ID NO. 227), ERHEKH(C) (SEQ ID NO. 228), EVWHRLQ(C) (SEQ ID NO. 229), ELWHRYP(C) (SEQ ID NO. 230), ELWHRAF(C) (SEQ ID NO. 231),
SGEYVFH(C) (SEQ ID NO. 307), SGQLKFP(C) (SEQ ID NO. 308), SGQIWFR(C) (SEQ ID NO. 309), SGEIHFN(C) (SEQ ID NO. 310), GQIWFIS(C) (SEQ ID NO. 311), GQIIFQS(C) (SEQ ID NO. 312), GQIRFDH(C) (SEQ ID NO. 313), GEMWFAL(C) (SEQ ID NO. 314), GELQFPP(C) (SEQ ID NO. 315), GELWFP(C) (SEQ ID NO. 316), GEMQFFI(C) (SEQ ID NO. 317), GELYFRA(C) (SEQ ID NO. 318), GEIRFAL(C) (SEQ ID NO. 319), GMIVFPH(C) (SEQ ID NO. 320), GEIWFEG(C) (SEQ ID NO. 321), GDLKFPL(C) (SEQ ID NO. 322), GQILFPV(C) (SEQ ID NO. 323), GELFFPK(C) (SEQ ID NO. 324), GQIMFPR(C) (SEQ ID NO. 325), GSLFFWP(C) (SEQ ID NO. 326), GEILFGM(C) (SEQ ID NO. 327), GQLKFPF(C) (SEQ ID NO. 328), GTIFFRD(C) (SEQ ID NO. 329), GQIKFAQ(C) (SEQ ID NO. 330), GTLIFHH(C) (SEQ ID NO. 331), GEIRFGS(C) (SEQ ID NO. 332), GQIQFPL(C) (SEQ ID NO. 333), GEIKFDH(C) (SEQ ID NO. 334), GEIQFGA(C) (SEQ ID NO. 335), GELFFEK(C) (SEQ ID NO. 336), GEIRFEL(C) (SEQ ID NO. 337), GEIYFER(C) (SEQ ID NO. 338), SGEIYFER(C) (SEQ ID NO. 339), AGEIYFER(C) (SEQ ID NO. 340), (C)GEIYFER (SEQ ID NO. 341),
SHTRLYF(C) (SEQ ID NO. 343), HMRLFFN(C) (SEQ ID NO. 344), SHQRLWF(C) (SEQ ID NO. 345), HQKMIFA(C) (SEQ ID NO. 346), HMRMYFE(C) (SEQ ID NO. 347), THQRLWF(C) (SEQ ID NO. 348), or HQKMIF(C) (SEQ ID NO. 349).
